SEQUENCE LISTING (77 sequences)

SEQ ID NO: 1 - protein, 9 aa, Artificial Sequence
  Class I 9-mer C962R_607
ELDARLWIM

SEQ ID NO: 2 - protein, 9 aa, Artificial Sequence
  Class I 9-mer I215L_191
EMEDDTYIL

SEQ ID NO: 3 - protein, 9 aa, Artificial Sequence
  Class I 9-mer C475L_292
YMDYSPPIF

SEQ ID NO: 4 - protein, 9 aa, Artificial Sequence
  Class I 9-mer B475L_63
MLDSFYKYF

SEQ ID NO: 5 - protein, 9 aa, Artificial Sequence
  Class I 9-mer C257L_197
QMEIKRFIK

SEQ ID NO: 6 - protein, 9 aa, Artificial Sequence
  Class I 9-mer NP1450L_334
HLDEVGYPI

SEQ ID NO: 7 - protein, 9 aa, Artificial Sequence
  Class I 9-mer CP2475L_1032
MMNQTNYSI

SEQ ID NO: 8 - protein, 9 aa, Artificial Sequence
  Class I 9-mer B646L_455
LMSALKWPI

SEQ ID NO: 9 - protein, 9 aa, Artificial Sequence
  Class I 9-mer F778R_273
YLELWHQDI

SEQ ID NO: 10 - protein, 9 aa, Artificial Sequence
  Class I 9-mer B407L_267
TIDSSMQEI

SEQ ID NO: 11 - protein, 9 aa, Artificial Sequence
  Class I 9-mer F334L_262
MLFQYIRYF

SEQ ID NO: 12 - protein, 9 aa, Artificial Sequence
  Class I 9-mer B646L_579
AMMITFALK

SEQ ID NO: 13 - protein, 9 aa, Artificial Sequence
  Class I 9-mer B318L_36
ALFHGIHPL

SEQ ID NO: 14 - protein, 9 aa, Artificial Sequence
  Class I 9-mer MGF_505_11L_120
ELFEFFHLF

SEQ ID NO: 15 - protein, 9 aa, Artificial Sequence
  Class I 9-mer NP868R_213
TLISPNHLM

SEQ ID NO: 16 - protein, 9 aa, Artificial Sequence
  Class I 9-mer EP424R_264
NMILKHYTL

SEQ ID NO: 17 - protein, 9 aa, Artificial Sequence
  Class I 9-mer NP1450_325
STICGNSDL

SEQ ID NO: 18 - protein, 9 aa, Artificial Sequence
  Class I 9-mer K145R_51
QLFLTMYKL

SEQ ID NO: 19 - protein, 9 aa, Artificial Sequence
  Class I 9-mer MGF_300_1L_177
SLSTLYCIF

SEQ ID NO: 20 - protein, 9 aa, Artificial Sequence
  Class I 9-mer G1211R_852
QTDAWRPDK

SEQ ID NO: 21 - protein, 9 aa, Artificial Sequence
  Class I 9-mer EP1242L_663
WLDVGRLTR

SEQ ID NO: 22 - protein, 9 aa, Artificial Sequence
  Class I 9-mer B438L_21
ESDLPRHNR

SEQ ID NO: 23 - protein, 9 aa, Artificial Sequence
  Class I 9-mer I267L_220
MVDNHPFKK

SEQ ID NO: 24 - protein, 9 aa, Artificial Sequence
  Class I 9-mer B962L_152
MTDDEIASR

SEQ ID NO: 25 - protein, 9 aa, Artificial Sequence
  Class I 9-mer CP2475L_2125
SLYPTQFDY

SEQ ID NO: 26 - protein, 9 aa, Artificial Sequence
  Class I 9-mer M1249L_580
QLYAVIYIY

SEQ ID NO: 27 - protein, 9 aa, Artificial Sequence
  Class I 9-mer DP79L_64
SIYIMVVEY

SEQ ID NO: 28 - protein, 9 aa, Artificial Sequence
  Class I 9-mer F778R_99
AIFDSYIDY

SEQ ID NO: 29 - protein, 9 aa, Artificial Sequence
  Class I 9-mer P1192R_1030
EILYAWLPY

SEQ ID NO: 30 - protein, 9 aa, Artificial Sequence
  Class I 9-mer D339L_321
KRHENIWML

SEQ ID NO: 31 - protein, 9 aa, Artificial Sequence
  Class I 9-mer MGF_505_6R_305
SRKTLNLLL

SEQ ID NO: 32 - protein, 9 aa, Artificial Sequence
  Class I 9-mer EP1242L_384
VRKLRFLGL

SEQ ID NO: 33 - protein, 9 aa, Artificial Sequence
  Class I 9-mer B438L_167
TRFSEHTKF

SEQ ID NO: 34 - protein, 9 aa, Artificial Sequence
  Class I 9-mer B175L_144
MRKEYGGKL

SEQ ID NO: 35 - protein, 9 aa, Artificial Sequence
  Class I 9-mer A859L_552
SEVAYPINW

SEQ ID NO: 36 - protein, 9 aa, Artificial Sequence
  Class I 9-mer I215L_71
SEMWHPNIY

SEQ ID NO: 37 - protein, 9 aa, Artificial Sequence
  Class I 9-mer S183L_25
AELESVNYY

SEQ ID NO: 38 - protein, 9 aa, Artificial Sequence
  Class I 9-mer B385R_44
QEYFNFLMI

SEQ ID NO: 39 - protein, 9 aa, Artificial Sequence
  Class I 9-mer B125R_102
EEYLRMHFI

SEQ ID NO: 40 - protein, 17 aa, Artificial Sequence
  Class II 9-mer Cluster B119L_49
HHAFSYLTKNPLTLNNS

SEQ ID NO: 41 - protein, 19 aa, Artificial Sequence
  Class II 9-mer Cluster B354L_250
KNAFVSIFTNASICMSNFS

SEQ ID NO: 42 - protein, 21 aa, Artificial Sequence
  Class II 9-mer Cluster B646L_322
KLRFWFNENVNLAIPSVSIPF

SEQ ID NO: 43 - protein, 17 aa, Artificial Sequence
  Class II 9-mer Cluster B962L_200
RIPFVILTSATIDTHKY

SEQ ID NO: 44 - protein, 18 aa, Artificial Sequence
  Class II 9-mer Cluster B962L_68
VHVFRILRNENTHSFQKY

SEQ ID NO: 45 - protein, 28 aa, Artificial Sequence
  Class II 9-mer Cluster C717R_549
AIDFYAIARNLRSMLSLDYLHTSEVKRN

SEQ ID NO: 46 - protein, 20 aa, Artificial Sequence
  Class II 9-mer Cluster CP2475L_551
YYYYVAQIYSNLTHNKQEFQ

SEQ ID NO: 47 - protein, 19 aa, Artificial Sequence
  Class II 9-mer Cluster E248R_69
EENFITNLSNQITQNLKDQ

SEQ ID NO: 48 - protein, 22 aa, Artificial Sequence
  Class II 9-mer Cluster E301R_44
PLIFKNLFIYFKNLKSKNILVR

SEQ ID NO: 49 - protein, 18 aa, Artificial Sequence
  Class II 9-mer Cluster E301R_253
NVKIAHIKSLASAMVTDK

SEQ ID NO: 50 - protein, 19 aa, Artificial Sequence
  Class II 9-mer Cluster EP1242L_23
EADMLSFISAAVNSTGLIG

SEQ ID NO: 51 - protein, 23 aa, Artificial Sequence
  Class II 9-mer Cluster EP1242L_459
QRNIIEAFSAALSKNTASDLNRS

SEQ ID NO: 52 - protein, 19 aa, Artificial Sequence
  Class II 9-mer Cluster EP364R_111
HVAYASIITAMTHLMVRDH

SEQ ID NO: 53 - protein, 17 aa, Artificial Sequence
  Class II 9-mer Cluster EP424R_267
LKHYTLNHAFTLSLICV

SEQ ID NO: 54 - protein, 18 aa, Artificial Sequence
  Class II 9-mer Cluster F165R_76
NTDVVYLIPSLTLHTPMF

SEQ ID NO: 55 - protein, 17 aa, Artificial Sequence
  Class II 9-mer Cluster F317L_237
LDIFMMLTSRRSLVNPW

SEQ ID NO: 56 - protein, 18 aa, Artificial Sequence
  Class II 9-mer Cluster G1211R_1023
ADLYKEFFNNTTNPIESF

SEQ ID NO: 57 - protein, 17 aa, Artificial Sequence
  Class II 9-mer Cluster G1340L_134
RQHIFFFQNNALQVTYR

SEQ ID NO: 58 - protein, 24 aa, Artificial Sequence
  Class II 9-mer Cluster G1340L_1145
FLLIEFFDVLYGLQSNSTRKHIEN

SEQ ID NO: 59 - protein, 19 aa, Artificial Sequence
  Class II 9-mer Cluster K145R_29
EKEFITLLNQALASTQLYR

SEQ ID NO: 60 - protein, 18 aa, Artificial Sequence
  Class II 9-mer Cluster K145R_72
KQEYLCLLINPKLVTKFL

SEQ ID NO: 61 - protein, 17 aa, Artificial Sequence
  Class II 9-mer Cluster NP1450L_337
EVGYPISFARTLQVAET

SEQ ID NO: 62 - protein, 24 aa, Artificial Sequence
  Class II 9-mer Cluster NP1450L_1281
QGKLVRLDNIYAIKTNGTNIFGAM

SEQ ID NO: 63 - protein, 16 aa, Artificial Sequence
  Class II 9-mer Cluster NP868R_592
KTGIYRAQTALISFIK

SEQ ID NO: 64 - protein, 19 aa, Artificial Sequence
  Class II 9-mer Cluster P1192R_1119
QGCYTYILSLQARELLIAA

SEQ ID NO: 65 - protein, 27 aa, Artificial Sequence
  Class II 9-mer Cluster PEP402R_328_BURMAKINA
YSPPKPLPSIPLLPNIPPLSTQNISLI

SEQ ID NO: 66 - protein, 351 aa, Artificial Sequence
  Class I ASFV Construct Before Vaccine CAD
MLFQYIRYFELFEFFHLFMLDSFYKYFWLDVGRLTRALFHGIHPLSLYPTQFDYMVDNHPFKKELDARLWIMMMNQTNYSISLSTLYCIFKRHENIWMLEMEDDTYILQLFLTMYKLYMDYSPPIFVRKLRFLGLSRKTLNLLLMRKEYGGKLQLYAVIYIYTRFSEHTKFAMMITFALKMTDDEIASRNMILKHYTLSIYIMVVEYTLISPNHLMQMEIKRFIKSTICGNSDLHLDEVGYPIQTDAWRPDKAIFDSYIDYEILYAWLPYLMSALKWPIYLELWHQDITIDSSMQEIESDLPRHNRSEMWHPNIYAELESVNYYSEVAYPINWQEYFNFLMIEEYLRMHFI

SEQ ID NO: 67 - protein, 399 aa, Artificial Sequence
  Class I ASFV Construct After Vaccine CAD
MLDSFYKYFAAYQMEIKRFIKAAYSIYIMVVEYAAYQEYFNFLMIAAYYMDYSPPIFQLFLTMYKLAAYMLFQYIRYFESDLPRHNRTRFSEHTKFQTDAWRPDKALFHGIHPLEEYLRMHFIAAYAELESVNYYTIDSSMQEIMTDDEIASRSTICGNSDLTLISPNHLMHLDEVGYPIKRHENIWMLAAYSEMWHPNIYSEVAYPINWSRKTLNLLLAAYEMEDDTYILELDARLWIMAAYMMNQTNYSIAAYSLSTLYCIFAAYMVDNHPFKKAIFDSYIDYWLDVGRLTRAAYEILYAWLPYSLYPTQFDYLMSALKWPIVRKLRFLGLMRKEYGGKLAAYAMMITFALKAAYNMILKHYTLAAYELFEFFHLFYLELWHQDIAAYQLYAVIYIY

SEQ ID NO: 68 - DNA, 1197 bp, Artificial Sequence
  Class I ASFV Construct After Vaccine CAD Back-Translation
atgctggacagcttctacaagtacttcgccgcctaccagatggaaatcaagcggttcatcaaggccgcctacagcatctacatcatggtggtggaatacgccgcctatcaagagtacttcaacttcctcatgattgccgcctactacatggactacagccctccaatcttccagctgtttctgaccatgtacaagctggccgcttacatgctgttccagtacatccggtacttcgagagcgatctgccccggcacaaccggacaagattcagcgagcacaccaagttccagaccgacgcttggaggcccgacaaggctctgtttcacggcattcaccctctggaagagtatctgcggatgcactttatcgccgcttacgccgagctggaaagcgtgaactactacaccatcgacagcagcatgcaagagatcatgaccgacgacgagatcgccagcagaagcaccatctgcggcaacagcgatctgacactgatcagccccaaccatctgatgcatctggacgaagtgggctaccccatcaagcggcacgagaacatttggatgctggccgcctattccgagatgtggcaccccaacatctacagcgaagtggcctatcctatcaactggtcccgcaagacactgaatctgctgctggctgcctacgaaatggaagatgacacttacatcctcgagctggacgcccggctgtggatcatggcagcctacatgatgaaccagaccaactactctatcgctgcctactctctgagcacactgtactgcatctttgctgcctacatggtggacaatcaccccttcaagaaggccatcttcgacagctacatcgactactggctggacgtgggcagactgaccagagccgcctatgagattctgtacgcttggctgccctattctctgtaccccacacagttcgactatctgatgagcgctctgaagtggcccatcgtgcggaagctgagatttctgggactgatgcggaaagagtacggcggcaaactggccgcatacgccatgatgatcacattcgcactgaaggctgcttacaacatgatcctcaagcactacaccctcgcagcctacgagctgttcgagttcttccatctgttctatctggaactgtggcaccaagatattgccgcttaccagctgtacgccgtgatctacatctac

SEQ ID NO: 69 - DNA, 5067 bp, Artificial Sequence
  Class I Plasmid
gctagcaccgttggtttccgtagtgtagtggttatcacgttcgcctaacacgcgaaaggtccccggttcgaaaccgggcactacaaaccaacaacgttaaaaaacaggtcctccccatactctttcattgtacacaccgcaagctcgacaatcatcggattgaagcattgtcgcacacatcttccacacaggatcagtacctgctttcgcttttaaccaaggcttttctccaagggatatttatagtctcaaaacacacaattactttacagttagggtgagtttccttttgtgctgttttttaaaataataatttagtatttgtatctcttatagaaatccaagcctatcatgtaaaatgtagctagtattaaaaagaacagattatctgtcttttatcgcacattaagcctctatagttactaggaaatattatatgcaaattaaccggggcaggggagtagccgagcttctcccacaagtctgtgcgagggggccggcgcgggcctagagatggcggcgtcggatcggccagcccgcctaatgagcgggcttttttttcttagggtgcaaaaggagagcctgtaagcgggcactcttccgtggtctggtggataaattcgcaagggtatcatggcggacgaccggggttcgagccccgtatccggccgtccgccgtgatccatgcggttaccgcccgcgtgtcgaacccaggtgtgcgacgtcagacaacgggggagtgctccttttggcttccttcccctaccggtctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcctgcaaaccacgttgtggtagaattggtaaagagagtcgtgtaaaatatcgagttcgcacatcttgttgtctgattattgatttttggcgaaaccatttgatcatatgacaagatgtgtatctaccttaacttaatgattttgataaaaatcattaggtacccctgatcactgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctagttctctcgttaacttaatgagacagatagaaactggtcttgtagaaacagagtagtcgcctgcttttctgccaggtgctgacttctctcccctgggcttttttctttttctcaggttgaaaagaagaagacgaagaagacgaagaagacaaaccgtcgtcgagaagccaccatgcagattttcgtcaagaccctgacgggcaagaccatcactcttgaggtcgagcccagtgacaccatcgagaatgtcaaggccaagatccaagacaaggaaggcatcccacctgaccagcagaggctgatattcgcgggcaaacagctggaggatggccgcaccctgtccgactacaacatccagaaagagtccaccttgcacctggtgctgcgtctgcgcggtgccgtcgacatgctggacagcttctacaagtacttcgccgcctaccagatggaaatcaagcggttcatcaaggccgcctacagcatctacatcatggtggtggaatacgccgcctatcaagagtacttcaacttcctcatgattgccgcctactacatggactacagccctccaatcttccagctgtttctgaccatgtacaagctggccgcttacatgctgttccagtacatccggtacttcgagagcgatctgccccggcacaaccggacaagattcagcgagcacaccaagttccagaccgacgcttggaggcccgacaaggctctgtttcacggcattcaccctctggaagagtatctgcggatgcactttatcgccgcttacgccgagctggaaagcgtgaactactacaccatcgacagcagcatgcaagagatcatgaccgacgacgagatcgccagcagaagcaccatctgcggcaacagcgatctgacactgatcagccccaaccatctgatgcatctggacgaagtgggctaccccatcaagcggcacgagaacatttggatgctggccgcctattccgagatgtggcaccccaacatctacagcgaagtggcctatcctatcaactggtcccgcaagacactgaatctgctgctggctgcctacgaaatggaagatgacacttacatcctcgagctggacgcccggctgtggatcatggcagcctacatgatgaaccagaccaactactctatcgctgcctactctctgagcacactgtactgcatctttgctgcctacatggtggacaatcaccccttcaagaaggccatcttcgacagctacatcgactactggctggacgtgggcagactgaccagagccgcctatgagattctgtacgcttggctgccctattctctgtaccccacacagttcgactatctgatgagcgctctgaagtggcccatcgtgcggaagctgagatttctgggactgatgcggaaagagtacggcggcaaactggccgcatacgccatgatgatcacattcgcactgaaggctgcttacaacatgatcctcaagcactacaccctcgcagcctacgagctgttcgagttcttccatctgttctatctggaactgtggcaccaagatattgccgcttaccagctgtacgccgtgatctacatctactaaagatctttttccctctgccaaaaattatggggacatcatgaagccccttgagcatctgacttctggctaataaaggaaatttattttcattgcaatagtgtgttggaattttttgtgtctctcactcggaaggacataagggcggcc

SEQ ID NO: 70 - protein, 511 aa, Artificial Sequence
  Class II ASFV Construct Before Vaccine CAD
AIDFYAIARNLRSMLSLDYLHTSEVKRNFLLIEFFDVLYGLQSNSTRKHIENRQHIFFFQNNALQVTYRQRNIIEAFSAALSKNTASDLNRSKLRFWFNENVNLAIPSVSIPFQGKLVRLDNIYAIKTNGTNIFGAMEENFITNLSNQITQNLKDQYYYYVAQIYSNLTHNKQEFQHVAYASIITAMTHLMVRDHLDIFMMLTSRRSLVNPWYSPPKPLPSIPLLPNIPPLSTQNISLIQGCYTYILSLQARELLIAANTDVVYLIPSLTLHTPMFEKEFITLLNQALASTQLYRPLIFKNLFIYFKNLKSKNILVRVHVFRILRNENTHSFQKYEADMLSFISAAVNSTGLIGKNAFVSIFTNASICMSNFSEVGYPISFARTLQVAETKQEYLCLLINPKLVTKFLADLYKEFFNNTTNPIESFNVKIAHIKSLASAMVTDKRIPFVILTSATIDTHKYHHAFSYLTKNPLTLNNSKTGIYRAQTALISFIKLKHYTLNHAFTLSLICV

SEQ ID NO: 71 - protein, 511 aa, Artificial Sequence
  Class II ASFV Construct After Vaccine CAD
QGKLVRLDNIYAIKTNGTNIFGAMAIDFYAIARNLRSMLSLDYLHTSEVKRNFLLIEFFDVLYGLQSNSTRKHIENKNAFVSIFTNASICMSNFSADLYKEFFNNTTNPIESFLDIFMMLTSRRSLVNPWKTGIYRAQTALISFIKQGCYTYILSLQARELLIAAYYYYVAQIYSNLTHNKQEFQEENFITNLSNQITQNLKDQPLIFKNLFIYFKNLKSKNILVRHHAFSYLTKNPLTLNNSYSPPKPLPSIPLLPNIPPLSTQNISLIEVGYPISFARTLQVAETEKEFITLLNQALASTQLYRVHVFRILRNENTHSFQKYRQHIFFFQNNALQVTYRKLRFWFNENVNLAIPSVSIPFRIPFVILTSATIDTHKYNVKIAHIKSLASAMVTDKEADMLSFISAAVNSTGLIGHVAYASIITAMTHLMVRDHLKHYTLNHAFTLSLICVKQEYLCLLINPKLVTKFLQRNIIEAFSAALSKNTASDLNRSNTDVVYLIPSLTLHTPMF

SEQ ID NO: 72 - DNA, 1533 bp, Artificial Sequence
  Class II ASFV Construct After Vaccine CAD Back-Translation
caaggcaagcttgtgcggctggacaacatctacgccatcaagaccaacggcaccaacatcttcggcgccatggccatcgacttctacgctatcgccagaaatctgcggagcatgctgtctctggactatctgcacaccagcgaagtgaagcggaactttctgctgatcgagttcttcgacgtgctgtacggactgcagagcaacagcacccggaagcacatcgagaacaagaacgccttcgtgtccatcttcaccaacgccagcatctgcatgagcaacttcagcgccgatctgtacaaagagttcttcaacaacaccaccaatccgatcgagagcttcctcgacatcttcatgatgctgaccagccggcggagcctcgtgaacccttggaaaaccggaatctacagagcccagaccgctctgatcagcttcatcaagcaaggctgctacacttacatcctcagcctccaagccagagagctgctgattgccgcctactactactatgtggcccaaatctacagcaatctgacccacaacaagcaagagttccaagaggaaaacttcatcaccaatctgagcaaccagatcacccagaatctgaaggaccagcctctgatcttcaagaatctgttcatctactttaagaatctcaagagcaagaacatcctcgtgcggcaccacgccttcagctatctgaccaagaatcctctgacactgaacaacagctacagccctcctaagcctctgcctagcatccctctgctgccaaacatccctccactgagcacccagaacatcagtctgatcgaagtgggctaccccatcagcttcgcccggacactgcaagtggccgagacagagaaagagttcatcacgctgctgaatcaagctctggccagcacacagctgtaccgggtgcacgtgttccggattctgcggaacgagaacacccacagcttccagaagtaccggcagcacatcttcttcttccaaaacaacgccctccaagtgacataccggaagctgcggttctggttcaacgagaatgtgaatctggctatcccctccgtgtctatcccctttcggatccccttcgtgattctgacaagcgccaccatcgacacccacaagtacaacgtgaagatcgcccacatcaagagtctggcctccgccatggtcaccgacaaagaggccgatatgctgagcttcatctctgccgccgtgaacagcaccggactgattggacatgtggcctacgcctccatcatcaccgccatgacacatctgatggtccgagatcatctgaagcactacacactgaaccatgccttcacactgtctctgatctgcgtgaagcaagaatatctgtgcctcctcatcaaccccaagctggtcaccaagtttctgcagcggaacatcatcgaggccttctccgccgctctgtccaagaatactgccagcgatctgaaccggtccaacaccgacgtggtgtatctgatcccctctctgactctgcacacacccatgttc

SEQ ID NO: 73 - DNA, 5246 bp, Artificial Sequence
  Class II Plasmid
gctagcaccgttggtttccgtagtgtagtggttatcacgttcgcctaacacgcgaaaggtccccggttcgaaaccgggcactacaaaccaacaacgttaaaaaacaggtcctccccatactctttcattgtacacaccgcaagctcgacaatcatcggattgaagcattgtcgcacacatcttccacacaggatcagtacctgctttcgcttttaaccaaggcttttctccaagggatatttatagtctcaaaacacacaattactttacagttagggtgagtttccttttgtgctgttttttaaaataataatttagtatttgtatctcttatagaaatccaagcctatcatgtaaaatgtagctagtattaaaaagaacagattatctgtcttttatcgcacattaagcctctatagttactaggaaatattatatgcaaattaaccggggcaggggagtagccgagcttctcccacaagtctgtgcgagggggccggcgcgggcctagagatggcggcgtcggatcggccagcccgcctaatgagcgggcttttttttcttagggtgcaaaaggagagcctgtaagcgggcactcttccgtggtctggtggataaattcgcaagggtatcatggcggacgaccggggttcgagccccgtatccggccgtccgccgtgatccatgcggttaccgcccgcgtgtcgaacccaggtgtgcgacgtcagacaacgggggagtgctccttttggcttccttcccctaccggtctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcctgcaaaccacgttgtggtagaattggtaaagagagtcgtgtaaaatatcgagttcgcacatcttgttgtctgattattgatttttggcgaaaccatttgatcatatgacaagatgtgtatctaccttaacttaatgattttgataaaaatcattaggtacccctgatcactgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctagttctctcgttaacttaatgagacagatagaaactggtcttgtagaaacagagtagtcgcctgcttttctgccaggtgctgacttctctcccctgggcttttttctttttctcaggttgaaaagaagaagacgaagaagacgaagaagacaaagccgccaccatggatgcaatgaagagagggctctgctgtgtgctgctgctgtgtggagcagtcttcgtttcgcccagcggtaccggatccgtcgaccaaggcaagcttgtgcggctggacaacatctacgccatcaagaccaacggcaccaacatcttcggcgccatggccatcgacttctacgctatcgccagaaatctgcggagcatgctgtctctggactatctgcacaccagcgaagtgaagcggaactttctgctgatcgagttcttcgacgtgctgtacggactgcagagcaacagcacccggaagcacatcgagaacaagaacgccttcgtgtccatcttcaccaacgccagcatctgcatgagcaacttcagcgccgatctgtacaaagagttcttcaacaacaccaccaatccgatcgagagcttcctcgacatcttcatgatgctgaccagccggcggagcctcgtgaacccttggaaaaccggaatctacagagcccagaccgctctgatcagcttcatcaagcaaggctgctacacttacatcctcagcctccaagccagagagctgctgattgccgcctactactactatgtggcccaaatctacagcaatctgacccacaacaagcaagagttccaagaggaaaacttcatcaccaatctgagcaaccagatcacccagaatctgaaggaccagcctctgatcttcaagaatctgttcatctactttaagaatctcaagagcaagaacatcctcgtgcggcaccacgccttcagctatctgaccaagaatcctctgacactgaacaacagctacagccctcctaagcctctgcctagcatccctctgctgccaaacatccctccactgagcacccagaacatcagtctgatcgaagtgggctaccccatcagcttcgcccggacactgcaagtggccgagacagagaaagagttcatcacgctgctgaatcaagctctggccagcacacagctgtaccgggtgcacgtgttccggattctgcggaacgagaacacccacagcttccagaagtaccggcagcacatcttcttcttccaaaacaacgccctccaagtgacataccggaagctgcggttctggttcaacgagaatgtgaatctggctatcccctccgtgtctatcccctttcggatccccttcgtgattctgacaagcgccaccatcgacacccacaagtacaacgtgaagatcgcccacatcaagagtctggcctccgccatggtcaccgacaaagaggccgatatgctgagcttcatctctgccgccgtgaacagcaccggactgattggacatgtggcctacgcctccatcatcaccgccatgacacatctgatggtccgagatcatctgaagcactacacactgaaccatgccttcacactgtctctgatctgcgtgaagcaagaatatctgtgcctcctcatcaaccccaagctggtcaccaagtttctgcagcggaacatcatcgaggccttctccgccgctctgtccaagaatactgccagcgatctgaaccggtccaacaccgacgtggtgtatctgatcccctctctgactctgcacacacccatgttctaaagatctttttccctctgccaaaaattatggggacatcatgaagccccttgagcatctgacttctggctaataaaggaaatttattttcattgcaatagtgtgttggaattttttgtgtctctcactcggaaggacataagggcggcc

SEQ ID NO: 74 - RNA, 1197 nt, Artificial Sequence
  Class I ASFV Construct After Vaccine CAD Back-Translation
augcuggacagcuucuacaaguacuucgccgccuaccagauggaaaucaagcgguucaucaaggccgccuacagcaucuacaucauggugguggaauacgccgccuaucaagaguacuucaacuuccucaugauugccgccuacuacauggacuacagcccuccaaucuuccagcuguuucugaccauguacaagcuggccgcuuacaugcuguuccaguacauccgguacuucgagagcgaucugccccggcacaaccggacaagauucagcgagcacaccaaguuccagaccgacgcuuggaggcccgacaaggcucuguuucacggcauucacccucuggaagaguaucugcggaugcacuuuaucgccgcuuacgccgagcuggaaagcgugaacuacuacaccaucgacagcagcaugcaagagaucaugaccgacgacgagaucgccagcagaagcaccaucugcggcaacagcgaucugacacugaucagccccaaccaucugaugcaucuggacgaagugggcuaccccaucaagcggcacgagaacauuuggaugcuggccgccuauuccgagauguggcaccccaacaucuacagcgaaguggccuauccuaucaacuggucccgcaagacacugaaucugcugcuggcugccuacgaaauggaagaugacacuuacauccucgagcuggacgcccggcuguggaucauggcagccuacaugaugaaccagaccaacuacucuaucgcugccuacucucugagcacacuguacugcaucuuugcugccuacaugguggacaaucaccccuucaagaaggccaucuucgacagcuacaucgacuacuggcuggacgugggcagacugaccagagccgccuaugagauucuguacgcuuggcugcccuauucucuguaccccacacaguucgacuaucugaugagcgcucugaaguggcccaucgugcggaagcugagauuucugggacugaugcggaaagaguacggcggcaaacuggccgcauacgccaugaugaucacauucgcacugaaggcugcuuacaacaugauccucaagcacuacacccucgcagccuacgagcuguucgaguucuuccaucuguucuaucuggaacuguggcaccaagauauugccgcuuaccagcuguacgccgugaucuacaucuac

SEQ ID NO: 75 - RNA, 5067 nt, Artificial Sequence
  Class I Plasmid
gcuagcaccguugguuuccguaguguagugguuaucacguucgccuaacacgcgaaagguccccgguucgaaaccgggcacuacaaaccaacaacguuaaaaaacagguccuccccauacucuuucauuguacacaccgcaagcucgacaaucaucggauugaagcauugucgcacacaucuuccacacaggaucaguaccugcuuucgcuuuuaaccaaggcuuuucuccaagggauauuuauagucucaaaacacacaauuacuuuacaguuagggugaguuuccuuuugugcuguuuuuuaaaauaauaauuuaguauuuguaucucuuauagaaauccaagccuaucauguaaaauguagcuaguauuaaaaagaacagauuaucugucuuuuaucgcacauuaagccucuauaguuacuaggaaauauuauaugcaaauuaaccggggcaggggaguagccgagcuucucccacaagucugugcgagggggccggcgcgggccuagagauggcggcgucggaucggccagcccgccuaaugagcgggcuuuuuuuucuuagggugcaaaaggagagccuguaagcgggcacucuuccguggucugguggauaaauucgcaaggguaucauggcggacgaccgggguucgagccccguauccggccguccgccgugauccaugcgguuaccgcccgcgugucgaacccaggugugcgacgucagacaacgggggagugcuccuuuuggcuuccuuccccuaccggucugccucgcgcguuucggugaugacggugaaaaccucugacacaugcagcucccggagacggucacagcuugucuguaagcggaugccgggagcagacaagcccgucagggcgcgucagcggguguuggcgggugucggggcgcagccaugacccagucacguagcgauagcggaguguauacuggcuuaacuaugcggcaucagagcagauuguacugagagugcaccauaugcggugugaaauaccgcacagaugcguaaggagaaaauaccgcaucaggcgcucuuccgcuuccucgcucacugacucgcugcgcucggucguucggcugcggcgagcgguaucagcucacucaaaggcgguaauacgguuauccacagaaucaggggauaacgcaggaaagaacaugugagcaaaaggccagcaaaaggccaggaaccguaaaaaggccgcguugcuggcguuuuuccauaggcuccgccccccugacgagcaucacaaaaaucgacgcucaagucagagguggcgaaacccgacaggacuauaaagauaccaggcguuucccccuggaagcucccucgugcgcucuccuguuccgacccugccgcuuaccggauaccuguccgccuuucucccuucgggaagcguggcgcuuucucauagcucacgcuguagguaucucaguucgguguaggucguucgcuccaagcugggcugugugcacgaaccccccguucagcccgaccgcugcgccuuauccgguaacuaucgucuugaguccaacccgguaagacacgacuuaucgccacuggcagcagccacugguaacaggauuagcagagcgagguauguaggcggugcuacagaguucuugaagugguggccuaacuacggcuacacuagaagaacaguauuugguaucugcgcucugcugaagccaguuaccuucggaaaaagaguugguagcucuugauccggcaaacaaaccaccgcugguagcggugguuuuuuuguuugcaagcagcagauuacgcgcagaaaaaaaggaucucaagaagauccuuugaucuuuucuacggggucugacgcucaguggaacgaaaacucacguuaagggauuuuggucaugagauuaucaaaaaggaucuucaccuagauccuuuuaaauuaaaaaugaaguuuuaaaucaaucuaaaguauauaugaguaaacuuggucugacaguuaccaaugcuuaaucagugaggcaccuaucucagcgaucugucuauuucguucauccauaguugccugacuccugcaaaccacguugugguagaauugguaaagagagucguguaaaauaucgaguucgcacaucuuguugucugauuauugauuuuuggcgaaaccauuugaucauaugacaagauguguaucuaccuuaacuuaaugauuuugauaaaaaucauuagguaccccugaucacuguggaaugugugucaguuaggguguggaaaguccccaggcuccccagcaggcagaaguaugcaaagcaugcaucucaauuagucagcaaccagguguggaaaguccccaggcuccccagcaggcagaaguaugcaaagcaugcaucucaauuagucagcaaccauagucccgccccuaacuccgcccaucccgccccuaacuccgcccaguuacggggucauuaguucauagcccauauauggaguuccgcguuacauaacuuacgguaaauggcccgccuggcugaccgcccaacgacccccgcccauugacgucaauaaugacguauguucccauaguaacgccaauagggacuuuccauugacgucaauggguggaguauuuacgguaaacugcccacuuggcaguacaucaaguguaucauaugccaaguacgcccccuauugacgucaaugacgguaaauggcccgccuggcauuaugcccaguacaugaccuuaugggacuuuccuacuuggcaguacaucuacguauuagucaucgcuauuaccauggugaugcgguuuuggcaguacaucaaugggcguggauagcgguuugacucacggggauuuccaagucuccaccccauugacgucaaugggaguuuguuuuggcaccaaaaucaacgggacuuuccaaaaugucguaacaacuccgccccauugacgcaaaugggcgguaggcguguacggugggaggucuauauaagcagagcucguuuagugaaccgucagaucgccuggagacgccauccacgcuguuuugaccuccauagaagacaccgggaccgauccagccuccgcggcucgcaucucuccuucacgcgcccgccgcccuaccugaggccgccauccacgccgguugagucgcguucugccgccucccgccuguggugccuccugaacugcguccgccgucuagguaaguuuaaagcucaggucgagaccgggccuuuguccggcgcucccuuggagccuaccuagacucagccggcucuccacgcuuugccugacccugcuugcucaacucuaguucucucguuaacuuaaugagacagauagaaacuggucuuguagaaacagaguagucgccugcuuuucugccaggugcugacuucucuccccugggcuuuuuucuuuuucucagguugaaaagaagaagacgaagaagacgaagaagacaaaccgucgucgagaagccaccaugcagauuuucgucaagacccugacgggcaagaccaucacucuugaggucgagcccagugacaccaucgagaaugucaaggccaagauccaagacaaggaaggcaucccaccugaccagcagaggcugauauucgcgggcaaacagcuggaggauggccgcacccuguccgacuacaacauccagaaagaguccaccuugcaccuggugcugcgucugcgcggugccgucgacaugcuggacagcuucuacaaguacuucgccgccuaccagauggaaaucaagcgguucaucaaggccgccuacagcaucuacaucauggugguggaauacgccgccuaucaagaguacuucaacuuccucaugauugccgccuacuacauggacuacagcccuccaaucuuccagcuguuucugaccauguacaagcuggccgcuuacaugcuguuccaguacauccgguacuucgagagcgaucugccccggcacaaccggacaagauucagcgagcacaccaaguuccagaccgacgcuuggaggcccgacaaggcucuguuucacggcauucacccucuggaagaguaucugcggaugcacuuuaucgccgcuuacgccgagcuggaaagcgugaacuacuacaccaucgacagcagcaugcaagagaucaugaccgacgacgagaucgccagcagaagcaccaucugcggcaacagcgaucugacacugaucagccccaaccaucugaugcaucuggacgaagugggcuaccccaucaagcggcacgagaacauuuggaugcuggccgccuauuccgagauguggcaccccaacaucuacagcgaaguggccuauccuaucaacuggucccgcaagacacugaaucugcugcuggcugccuacgaaauggaagaugacacuuacauccucgagcuggacgcccggcuguggaucauggcagccuacaugaugaaccagaccaacuacucuaucgcugccuacucucugagcacacuguacugcaucuuugcugccuacaugguggacaaucaccccuucaagaaggccaucuucgacagcuacaucgacuacuggcuggacgugggcagacugaccagagccgccuaugagauucuguacgcuuggcugcccuauucucuguaccccacacaguucgacuaucugaugagcgcucugaaguggcccaucgugcggaagcugagauuucugggacugaugcggaaagaguacggcggcaaacuggccgcauacgccaugaugaucacauucgcacugaaggcugcuuacaacaugauccucaagcacuacacccucgcagccuacgagcuguucgaguucuuccaucuguucuaucuggaacuguggcaccaagauauugccgcuuaccagcuguacgccgugaucuacaucuacuaaagaucuuuuucccucugccaaaaauuauggggacaucaugaagccccuugagcaucugacuucuggcuaauaaaggaaauuuauuuucauugcaauaguguguuggaauuuuuugugucucucacucggaaggacauaagggcggcc

SEQ ID NO: 76 - RNA, 1533 nt, Artificial Sequence
  Class II ASFV Construct After Vaccine CAD Back-Translation
caaggcaagcuugugcggcuggacaacaucuacgccaucaagaccaacggcaccaacaucuucggcgccauggccaucgacuucuacgcuaucgccagaaaucugcggagcaugcugucucuggacuaucugcacaccagcgaagugaagcggaacuuucugcugaucgaguucuucgacgugcuguacggacugcagagcaacagcacccggaagcacaucgagaacaagaacgccuucguguccaucuucaccaacgccagcaucugcaugagcaacuucagcgccgaucuguacaaagaguucuucaacaacaccaccaauccgaucgagagcuuccucgacaucuucaugaugcugaccagccggcggagccucgugaacccuuggaaaaccggaaucuacagagcccagaccgcucugaucagcuucaucaagcaaggcugcuacacuuacauccucagccuccaagccagagagcugcugauugccgccuacuacuacuauguggcccaaaucuacagcaaucugacccacaacaagcaagaguuccaagaggaaaacuucaucaccaaucugagcaaccagaucacccagaaucugaaggaccagccucugaucuucaagaaucuguucaucuacuuuaagaaucucaagagcaagaacauccucgugcggcaccacgccuucagcuaucugaccaagaauccucugacacugaacaacagcuacagcccuccuaagccucugccuagcaucccucugcugccaaacaucccuccacugagcacccagaacaucagucugaucgaagugggcuaccccaucagcuucgcccggacacugcaaguggccgagacagagaaagaguucaucacgcugcugaaucaagcucuggccagcacacagcuguaccgggugcacguguuccggauucugcggaacgagaacacccacagcuuccagaaguaccggcagcacaucuucuucuuccaaaacaacgcccuccaagugacauaccggaagcugcgguucugguucaacgagaaugugaaucuggcuauccccuccgugucuauccccuuucggauccccuucgugauucugacaagcgccaccaucgacacccacaaguacaacgugaagaucgcccacaucaagagucuggccuccgccauggucaccgacaaagaggccgauaugcugagcuucaucucugccgccgugaacagcaccggacugauuggacauguggccuacgccuccaucaucaccgccaugacacaucugaugguccgagaucaucugaagcacuacacacugaaccaugccuucacacugucucugaucugcgugaagcaagaauaucugugccuccucaucaaccccaagcuggucaccaaguuucugcagcggaacaucaucgaggccuucuccgccgcucuguccaagaauacugccagcgaucugaaccgguccaacaccgacgugguguaucugauccccucucugacucugcacacacccauguuc

SEQ ID NO: 77 - RNA, 5246 nt, Artificial Sequence
  Class II Plasmid
gcuagcaccguugguuuccguaguguagugguuaucacguucgccuaacacgcgaaagguccccgguucgaaaccgggcacuacaaaccaacaacguuaaaaaacagguccuccccauacucuuucauuguacacaccgcaagcucgacaaucaucggauugaagcauugucgcacacaucuuccacacaggaucaguaccugcuuucgcuuuuaaccaaggcuuuucuccaagggauauuuauagucucaaaacacacaauuacuuuacaguuagggugaguuuccuuuugugcuguuuuuuaaaauaauaauuuaguauuuguaucucuuauagaaauccaagccuaucauguaaaauguagcuaguauuaaaaagaacagauuaucugucuuuuaucgcacauuaagccucuauaguuacuaggaaauauuauaugcaaauuaaccggggcaggggaguagccgagcuucucccacaagucugugcgagggggccggcgcgggccuagagauggcggcgucggaucggccagcccgccuaaugagcgggcuuuuuuuucuuagggugcaaaaggagagccuguaagcgggcacucuuccguggucugguggauaaauucgcaaggguaucauggcggacgaccgggguucgagccccguauccggccguccgccgugauccaugcgguuaccgcccgcgugucgaacccaggugugcgacgucagacaacgggggagugcuccuuuuggcuuccuuccccuaccggucugccucgcgcguuucggugaugacggugaaaaccucugacacaugcagcucccggagacggucacagcuugucuguaagcggaugccgggagcagacaagcccgucagggcgcgucagcggguguuggcgggugucggggcgcagccaugacccagucacguagcgauagcggaguguauacuggcuuaacuaugcggcaucagagcagauuguacugagagugcaccauaugcggugugaaauaccgcacagaugcguaaggagaaaauaccgcaucaggcgcucuuccgcuuccucgcucacugacucgcugcgcucggucguucggcugcggcgagcgguaucagcucacucaaaggcgguaauacgguuauccacagaaucaggggauaacgcaggaaagaacaugugagcaaaaggccagcaaaaggccaggaaccguaaaaaggccgcguugcuggcguuuuuccauaggcuccgccccccugacgagcaucacaaaaaucgacgcucaagucagagguggcgaaacccgacaggacuauaaagauaccaggcguuucccccuggaagcucccucgugcgcucuccuguuccgacccugccgcuuaccggauaccuguccgccuuucucccuucgggaagcguggcgcuuucucauagcucacgcuguagguaucucaguucgguguaggucguucgcuccaagcugggcugugugcacgaaccccccguucagcccgaccgcugcgccuuauccgguaacuaucgucuugaguccaacccgguaagacacgacuuaucgccacuggcagcagccacugguaacaggauuagcagagcgagguauguaggcggugcuacagaguucuugaagugguggccuaacuacggcuacacuagaagaacaguauuugguaucugcgcucugcugaagccaguuaccuucggaaaaagaguugguagcucuugauccggcaaacaaaccaccgcugguagcggugguuuuuuuguuugcaagcagcagauuacgcgcagaaaaaaaggaucucaagaagauccuuugaucuuuucuacggggucugacgcucaguggaacgaaaacucacguuaagggauuuuggucaugagauuaucaaaaaggaucuucaccuagauccuuuuaaauuaaaaaugaaguuuuaaaucaaucuaaaguauauaugaguaaacuuggucugacaguuaccaaugcuuaaucagugaggcaccuaucucagcgaucugucuauuucguucauccauaguugccugacuccugcaaaccacguugugguagaauugguaaagagagucguguaaaauaucgaguucgcacaucuuguugucugauuauugauuuuuggcgaaaccauuugaucauaugacaagauguguaucuaccuuaacuuaaugauuuugauaaaaaucauuagguaccccugaucacuguggaaugugugucaguuaggguguggaaaguccccaggcuccccagcaggcagaaguaugcaaagcaugcaucucaauuagucagcaaccagguguggaaaguccccaggcuccccagcaggcagaaguaugcaaagcaugcaucucaauuagucagcaaccauagucccgccccuaacuccgcccaucccgccccuaacuccgcccaguuacggggucauuaguucauagcccauauauggaguuccgcguuacauaacuuacgguaaauggcccgccuggcugaccgcccaacgacccccgcccauugacgucaauaaugacguauguucccauaguaacgccaauagggacuuuccauugacgucaauggguggaguauuuacgguaaacugcccacuuggcaguacaucaaguguaucauaugccaaguacgcccccuauugacgucaaugacgguaaauggcccgccuggcauuaugcccaguacaugaccuuaugggacuuuccuacuuggcaguacaucuacguauuagucaucgcuauuaccauggugaugcgguuuuggcaguacaucaaugggcguggauagcgguuugacucacggggauuuccaagucuccaccccauugacgucaaugggaguuuguuuuggcaccaaaaucaacgggacuuuccaaaaugucguaacaacuccgccccauugacgcaaaugggcgguaggcguguacggugggaggucuauauaagcagagcucguuuagugaaccgucagaucgccuggagacgccauccacgcuguuuugaccuccauagaagacaccgggaccgauccagccuccgcggcucgcaucucuccuucacgcgcccgccgcccuaccugaggccgccauccacgccgguugagucgcguucugccgccucccgccuguggugccuccugaacugcguccgccgucuagguaaguuuaaagcucaggucgagaccgggccuuuguccggcgcucccuuggagccuaccuagacucagccggcucuccacgcuuugccugacccugcuugcucaacucuaguucucucguuaacuuaaugagacagauagaaacuggucuuguagaaacagaguagucgccugcuuuucugccaggugcugacuucucuccccugggcuuuuuucuuuuucucagguugaaaagaagaagacgaagaagacgaagaagacaaagccgccaccauggaugcaaugaagagagggcucugcugugugcugcugcuguguggagcagucuucguuucgcccagcgguaccggauccgucgaccaaggcaagcuugugcggcuggacaacaucuacgccaucaagaccaacggcaccaacaucuucggcgccauggccaucgacuucuacgcuaucgccagaaaucugcggagcaugcugucucuggacuaucugcacaccagcgaagugaagcggaacuuucugcugaucgaguucuucgacgugcuguacggacugcagagcaacagcacccggaagcacaucgagaacaagaacgccuucguguccaucuucaccaacgccagcaucugcaugagcaacuucagcgccgaucuguacaaagaguucuucaacaacaccaccaauccgaucgagagcuuccucgacaucuucaugaugcugaccagccggcggagccucgugaacccuuggaaaaccggaaucuacagagcccagaccgcucugaucagcuucaucaagcaaggcugcuacacuuacauccucagccuccaagccagagagcugcugauugccgccuacuacuacuauguggcccaaaucuacagcaaucugacccacaacaagcaagaguuccaagaggaaaacuucaucaccaaucugagcaaccagaucacccagaaucugaaggaccagccucugaucuucaagaaucuguucaucuacuuuaagaaucucaagagcaagaacauccucgugcggcaccacgccuucagcuaucugaccaagaauccucugacacugaacaacagcuacagcccuccuaagccucugccuagcaucccucugcugccaaacaucccuccacugagcacccagaacaucagucugaucgaagugggcuaccccaucagcuucgcccggacacugcaaguggccgagacagagaaagaguucaucacgcugcugaaucaagcucuggccagcacacagcuguaccgggugcacguguuccggauucugcggaacgagaacacccacagcuuccagaaguaccggcagcacaucuucuucuuccaaaacaacgcccuccaagugacauaccggaagcugcgguucugguucaacgagaaugugaaucuggcuauccccuccgugucuauccccuuucggauccccuucgugauucugacaagcgccaccaucgacacccacaaguacaacgugaagaucgcccacaucaagagucuggccuccgccauggucaccgacaaagaggccgauaugcugagcuucaucucugccgccgugaacagcaccggacugauuggacauguggccuacgccuccaucaucaccgccaugacacaucugaugguccgagaucaucugaagcacuacacacugaaccaugccuucacacugucucugaucugcgugaagcaagaauaucugugccuccucaucaaccccaagcuggucaccaaguuucugcagcggaacaucaucgaggccuucuccgccgcucuguccaagaauacugccagcgaucugaaccgguccaacaccgacgugguguaucugauccccucucugacucugcacacacccauguucuaaagaucuuuuucccucugccaaaaauuauggggacaucaugaagccccuugagcaucugacuucuggcuaauaaaggaaauuuauuuucauugcaauaguguguuggaauuuuuugugucucucacucggaaggacauaagggcggcc

(19) United States
(12) Patent Application Publication
(10) Pub. No.: US 2023/0192777 A1
(43) Pub. Date: Jun. 22, 2023

(54) EPITOPIC VACCINE FOR AFRICAN SWINE FEVER VIRUS

(71) Applicant: EpiVax, Inc., Providence, RI (US)

(72) Inventors: Lenny Moise, Providence, RI (US); Andres Gutierrez, Providence, RI (US); William Martin, Providence, RI (US); Anne De Groot, Providence, RI (US)

(21) Appl. No.: 17/783,461

(22) PCT Filed: Dec. 11, 2020
(86) PCT No.: PCT/US2020/064507

§ 371 Date: Jun. 8, 2022

Related U.S. Application Data
(60) Provisional application No. 62/946,714, filed on Dec. 11, 2019.
(60) Provisional application No. 63/034,567, filed on Jun. 4, 2020.

(51) Int. Cl. C07K 14/01; A61K 39/12; C12N 15/86; C07K 7/06

(52) U.S. Cl. CPC C07K 14/01; A61K 39/12; C07K 7/06; C12N 15/86; A61K 2039/53

(57) ABSTRACT

The present disclosure generally relates to a T-cell epitope compounds and compositions effective against African Swine Fever Virus (“ASFV”) and related diseases. Such T-cell epitope compounds and compositions include immunogenic T-cell epitope polypeptides (including concatemeric polypeptides and chimeric or fusion polypeptides), as well as nucleic acids, plasmids, vectors, and cells which express the polypeptides, pharmaceutical compositions, and vaccines, and the uses thereof. The present disclosure is particularly suited to produce vaccines for non-human animals, particularly for vaccinating swine against ASFV infection and related diseases.

### CROSS-REFERENCE TO RELATED APPLICATION

This application depends from and claims priority to both U.S. Provisional Application No: 62/946,714 filed Dec. 11, 2019 and U.S. Provisional Application No: 63/034,567 filed Jun. 4, 2020, the entire contents of each of which are incorporated herein by reference.

### REFERENCE TO SEQUENCE LISTING SUBMITTED ELECTRONICALLY

The instant application contains a Sequence Listing which has been submitted electronically in ASCII format and is hereby incorporated by reference in its entirety. Said ASCII copy, created Dec. 11, 2020, is named “EPV0023WO_Seq_Listing_ST25.txt” and is 65 KB bytes in size.

### FIELD

The present disclosure generally relates to a T-cell epitope compounds and compositions effective against African Swine Fever Virus (“ASFV”) and related diseases. Such T-cell epitope compounds and compositions include immunogenic T-cell epitope polypeptides (including concatemeric polypeptides and chimeric or fusion polypeptides), as well as nucleic acids, plasmids, vectors, and cells which express the polypeptides, pharmaceutical compositions, and vaccines, and the uses thereof. The present disclosure is particularly suited to produce vaccines for non-human animals, particularly for vaccinating swine against ASFV infection and related diseases.

### BACKGROUND

African swine fever virus (ASFV) is the etiological agent of African swine fever (ASF), a highly contagious hemorrhagic disease of swine that affects domestic pigs and wild boars of all ages and breeds. Several clinical forms of ASF are presented in swine and include a hyper-acute or acute disease, a sub-acute disease and a chronic disease with mortality rates ranging from 100% to 3% depending on the virulence of the viral isolate, route of infection, and the host. ASFV transmission to unexposed domestic pigs occurs by direct contact with an infected animal or the body fluids and carcasses of infected animals, by indirect contact with contaminated materials, or through the consumption of contaminated products.

ASFV is a large, enveloped, double-stranded DNA virus. It is the sole member of the Asfarviridae family, genus Asfivirus. Wild suids and soft ticks of the genus Ornithodoros are the natural reservoir for the ASF virus and represent a source for infection. Depending on the virus isolate, the ASFV genome length varies between 170 and 190 kbp which contains between 151 and 167 open reading frames (ORFs) and encodes 54 structural proteins and over 100 infection related proteins. 24 distinct genotypes of ASFV have been identified based on the sequencing of the gene encoding the major capsid protein p72. All genotypes are present in south-eastern Africa. Genotype I is predominantly present in West Africa but has also spread outside Africa to Europe, South America and the Caribbean. Genotype II has also reached Eastern Europe as well as Asia.

ASF poses a devastating threat to the global pig industry and has been spreading at an alarming rate in the past few years, affecting more than 55 countries in three different continents: Africa, Asia, and Europe. The introduction of ASF into these countries has dramatically impacted their socio-economics, pig production and status for international trade. Prevention, control, and eradication measures for ASF are mainly based on early detection and on the implementation of strict sanitary measures. However, successful control of ASF has proven to be challenging and the risk of introducing the virus into ASF-free countries is increasing. A vaccine against ASF is urgently needed to improve prevention and control strategies and to mitigate major economic losses in endemic and non-endemic areas.

No vaccine currently exists against ASF. The complexity of the virus and the large number of encoded proteins, with some involved in the modulation of host immune responses, has made it challenging to identify immunogenic targets and hindered the development of an efficacious ASF vaccine. Another challenge is the genetic diversity of the ASFV and the limited knowledge of antigens involved in conferring cross-protection. Thus far, little to no cross-protection has been reported from clinical trials. However, pigs that survive ASFV infection generate protection against subsequent infections with a homologous ASFV. Several efforts have been made to develop an ASF vaccine with a current focus on the induction of both humoral and cellular immune responses due to their potential role in conferring protection from ASF.

### SUMMARY

The aim of the present disclosure is to provide novel, therapeutic T-cell epitope compounds and compositions (including one or more of e.g., polypeptides having a sequence comprising, consisting of, or consisting essentially of one or more of SEQ ID NOS: 1-65 and/or fragments and variants thereof, and optionally 1 to 12 additional amino acids distributed in any ratio on the N terminus and/or C-terminus of the polypeptide of SEQ ID NOS: 1-65 as disclosed herein; concatemeric polypeptides as disclosed herein, including concatemeric polypeptides comprising, consisting of, or consisting essentially of one or more of SEQ ID NOS: 66-67 and 70-71 as disclosed herein; chimeric or fusion polypeptide compositions as disclosed herein; nucleic acids as disclosed herein, including nucleic acids having a sequence comprising, consisting of, or consisting essentially of one of more of SEQ ID NOS 68, 69, 72, 73-77 as discosed herein, expression cassettes, plasmids, expression vectors, recombinant viruses, or cells isolated, synthetic, and/or recombinant as disclosed herein; vaccine compositions or formulations as disclosed herein; and/or pharmaceutical compositions or formulations as disclosed herein), and use of the same, e.g., in methods of stimulating, inducing, and/or expanding an immune response to ASFV and/or associated diseases in a subject and methods of treating and/or preventing ASFV and/or associated diseases in a subject..

In aspects, a T-cell epitope compound or composition of the present disclosure includes one or more peptides or polypeptides as disclosed herein. In aspects, an epitopic composition includes one or more of polypeptides having a sequence comprising, consisting of, or consisting essentially of one or more of SEQ ID NOS: 1-65, and fragments thereof. The phrase “consisting essentially of” is intended to mean that a peptide or polypeptide according to the present disclosure, in addition to the sequence according to any of SEQ ID NOS: 1-65 or a fragment or variant thereof, contains additional amino acids or residues that may be present at either terminus of the peptide and/or on a side chain that are not necessarily forming part of the peptide or polypeptide that functions as an MHC ligand and provided they do not substantially impair the activity of the peptide to function as a T-cell epitope. The polypeptides of the present disclosure may be synthetic or recombinant, and may comprise post-transcriptional modifications such as glycosylation, added chemical groups, etc. In aspects, the peptides or polypeptides can be either in neutral (uncharged) or salt forms, and may be either free of or include modifications such as glycosylation, side chain oxidation, or phosphorylation. In aspects, the peptides or polypeptides of the instant disclosure can be capped with an n-terminal acetyl and/or c-terminal amino group.

In aspects, the instant disclosure is directed to a peptide or polypeptide comprising, consisting, or consisting essentially of an amino acid sequence of SEQ ID NOS: 1-65 (and/or fragments or variants thereof), and optionally 1 to 12 additional amino acids distributed in any ratio on the N terminus and/or C-terminus of the polypeptide of SEQ ID NOS: 1-65. In aspects, the instant disclosure is directed to a peptide or polypeptide having a core amino acid sequence comprising, consisting of, or consisting essentially of one or more peptides or polypeptides having an amino acid sequence of SEQ ID NOS: 1-65, and optionally having extensions of 1 to 12 amino acids on the C-terminal and/or the N-terminal of the core amino acid sequence, wherein the overall number of these flanking amino acids is 1 to 12, 1 to 3, 2 to 4, 3 to 6, 1 to 10, 1 to 8, 1 to 6, 2 to 12, 2 to 10, 2 to 8, 2 to 6, 3 to 12, 3 to 10, 3 to 8, 3 to 6, 4 to 12, 4 to 10, 4 to 8, 4 to 6, 5 to 12, 5 to 10, 5 to 8, 5 to 6, 6 to 12, 6 to 10, 6 to 8, 7 to 12, 7 to 10, 7 to 8, 8 to 12, 8 to 10, 9 to 12, 9 to 10, or 10 to 12, wherein the flanking amino acids can be distributed in any ratio to the C-terminus and the N-terminus (for example all flanking amino acids can be added to one terminus, or the amino acids can be added equally to both termini or in any other ratio). In aspects, the instant disclosure is directed to a peptide or polypeptide having a core sequence comprising, consisting of, or consisting essentially of one or more peptides or polypeptides having an amino acid sequence of SEQ ID NOS: 1-65 (and/or fragments and variants thereof), optionally with extensions of 1 to 12 amino acids on the C-terminal and/or the N-terminal, wherein the overall number of these flanking amino acids is 1 to 12, 1 to 3, 2 to 4, 3 to 6, 1 to 10, 1 to 8, 1 to 6, 2 to 12, 2 to 10, 2 to 8, 2 to 6, 3 to 12, 3 to 10, 3 to 8, 3 to 6, 4 to 12, 4 to 10, 4 to 8, 4 to 6, 5 to 12, 5 to 10, 5 to 8, 5 to 6, 6 to 12, 6 to 10, 6 to 8, 7 to 12, 7 to 10, 7 to 8, 8 to 12, 8 to 10, 9 to 12, 9 to 10, or 10 to 12, wherein the flanking amino acids can be distributed in any ratio to the C-terminus and the N-terminus (for example all flanking amino acids can be added to one terminus, or the amino acids can be added equally to both termini or in any other ratio), provided that the polypeptide with the flanking amino acids is still able to bind to a same HLA molecule (i.e., retain MHC binding propensity) as said polypeptide core sequence without said flanking amino acids. In aspects, said polypeptide with the flanking amino acids is still able to bind to a same HLA molecule (i.e., retain MHC binding propensity) and/or retain the same TCR specificity), and/or retain ASFV activity as said polypeptide core sequence without said flanking amino acids. In aspects, said flanking amino acid sequences are those that also flank the peptides or polypeptides included therein in the naturally occurring protein. For example, for a peptide or polypeptide having a core sequence comprising, consisting of, or consisting essentially of one or more peptides or polypeptides having an amino acid sequence of SEQ ID NOS: 1-65 (and/or fragments and variants thereof), optionally with extensions of 1 to 12 amino acids on the C-terminal and/or the N-terminal, the extensions of 1 to 12 amino acids are those found flanking the amino acid sequence of SEQ ID NOS: 1-65 in an encoded protein sequence of African swine fever virus (ASFV). In aspects, said flanking amino acid sequences as described herein may serve as a MHC-stabilizing region. The use of a longer peptide may allow endogenous processing by patient cells and may lead to more effective antigen presentation and induction of T-cell responses . In aspects, the peptides or polypeptides can be either in neutral (uncharged) or salt forms, and may be either free of or include modifications such as glycosylation, side chain oxidation, or phosphorylation. In certain aspects, the peptides or polypeptides of the instant disclosure can be capped with an n-terminal acetyl and/or c-terminal amino group.

In aspects, the present disclosure is directed to a concatemeric polypeptide that comprises at one or more of the instantly-disclosed polypeptides or peptides (e.g., but not limited to, a peptide or polypeptide comprising, consisting, or consisting essentially of an amino acid sequence of SEQ ID NOS: 1-65 (and/or fragments or variants thereof), and optionally 1 to 12 additional amino acids distributed in any ratio on the N terminus and/or C-terminus of the polypeptide of SEQ ID NOS: 1-65) linked, fused, or joined together (e.g., fused in-frame, chemically-linked, or otherwise bound) to an additional peptide or polypeptide. Such additional peptide or polypeptide may be one or more of the instantly instantly-disclosed polypeptides or peptides, or may be an additional peptide or polypeptide of interest. In aspects a concatemeric peptide is composed of 3 or more, 4 or more, 5 or more, 6 or more, 7 or more, 8 or more, or 9 or more of the instantly-disclosed peptides or polypeptides. In other aspects, the concatemeric peptides or polypeptides include 1000 or more, 1000 or less, 900 or less, 500 or less, 100 or less, 75 or less, 50 or less, 40 or less, 30 or less, 20 or less, or 100 or less peptide epitopes. In yet other embodiments, a concatemeric peptide has 3-100, 5-100, 10-100, 15-100, 20-100, 25-100, 30-100, 35-100, 40-100, 45-100, 50-100, 55-100, 60-100, 65-100, 70-100, 75-100, 80-100, 90-100, 5-50, 10-50, 15-50, 20-50, 25-50, 30-50, 35-50, 40-50, 45-50, 100-150, 100-200, 100-300, 100-400, 100-500, 50-500, 50-800, 50-1,000, or 100-1,000 of the instantly-disclosed peptides or polypeptides linked, fused, or joined together. Each peptide or polypeptide of the concatemeric polypeptide may optionally have one or more linkers, which may optionally be cleavage-sensitive sites, adjacent to their N- and/or C-terminal end. In such a concatemeric peptide, two or more of the peptide epitopes may have a cleavage-sensitive site between them. Alternatively, two or more of the peptide epitopes may be connected directly to one another or through a linker that is not a cleavage-sensitive site. In aspects, a concatemeric polypeptide comprises, consists of, or consists essentially of one or more of SEQ ID NOS: 66-67 and 70-71 (which in aspects may be isolated, synthetic, and/or recombinant). In aspects, the concatemeric polypeptides can be either in neutral (uncharged) or salt forms, and may be either free of or include modifications such as glycosylation, side chain oxidation, or phosphorylation. In certain aspects, the concatemeric polypeptides of the instant disclosure can be capped with an n-terminal acetyl and/or c-terminal amino group.

In aspects, one or more peptides or polypeptides or concatemeric polypeptides of the instant disclosure (e.g., but not limited to, a peptide or polypeptide comprising, consisting, or consisting essentially of an amino acid sequence of SEQ ID NOS: 1-65 (and/or fragments or variants thereof), and optionally 1 to 12 additional amino acids distributed in any ratio on the N terminus and/or C-terminus of the polypeptide of SEQ ID NOS: 1-65, or a concatemeric polypeptide comprising, consisting of, or consisting essentially of one or more of SEQ ID NOS: 66-67 and 70-71) is joined to, linked to (e.g., fused in-frame, chemically-linked, or otherwise bound), and/or inserted into a heterologous polypeptide. In aspects, the one or more peptides or polypeptides or concatemeric polypeptides of the instant disclosure may be joined to, linked to (e.g., fused in-frame, chemically-linked, or otherwise bound), and/or inserted into a heterologous polypeptide as a whole, although it may be made up from a joined to, linked to (e.g., fused in-frame, chemically-linked, or otherwise bound), and/or inserted amino acid sequence, together with flanking amino acids of the heterologous polypeptide.

In aspects, the present disclosure is directed to a chimeric or fusion polypeptide composition (which in aspects may be isolated, synthetic, and/or recombinant) comprising one or more peptides, polypeptides or concatemeric peptides of the present disclosure. In aspects, a chimeric or fusion polypeptide composition of the present disclosure comprises one or more peptides, polypeptides, and/or concatemeric peptides of the present disclosure joined to, linked to (e.g., fused in-frame, chemically-linked, or otherwise bound), and/or inserted into a heterologous polypeptide. In aspects, the one or more peptides, polypeptides, and/or concatemeric peptides of the present disclosure may be inserted into the heterologous polypeptide, may be added to the C-terminus (with or without the use of linkers, as is known in the art), and/or added to the N-terminus (with or without the use of linkers, as is known in the art) of the heterologous polypeptide. In aspects of the above chimeric or fusion polypeptide compositions, the one or more peptides, polypeptides, or concatemeric peptides may be joined to, linked to (e.g., fused in-frame, chemically-linked, or otherwise bound), and/or inserted into a heterologous polypeptide as a whole, although it may be made up from a joined to, linked to (e.g., fused in-frame, chemically-linked, or otherwise bound), and/or inserted amino acid sequence, together with flanking amino acids of the heterologous polypeptide. In aspects, a chimeric or fusion polypeptide composition of the present disclosure comprises a peptide, polypeptide, and/or concatemeric peptide of the instant disclosure, said peptide, polypeptide, and/or concatemeric peptide having a sequence that is not naturally included in the heterologous polypeptide and/or is not located at its natural position in the heterologous polypeptide. In aspects, the present disclosure is directed to polypeptide (which, in aspects, may be an isolated, synthetic, and/or recombinant) having a sequence comprising one or more of SEQ ID NOS: 1-67, 70-71, wherein said one or more of SEQ ID NOS: 1-67, 70-71 is not naturally included in the polypeptide and/or said one or more of SEQ ID NOS: 1-67, 70-71 is not located at its natural position in the polypeptide. In aspects, the one or more of peptide, polypeptide, and/or concatemeric peptide of the present disclosure can be joined, linked to (e.g., fused in-frame, chemically-linked, or otherwise bound), and/or inserted into the heterologous polypeptide. In aspects of above-described chimeric or fusion polypeptide compositions, the chimeric or fusion polypeptides may be isolated, synthetic, and/or recombinant.

In aspects, the instant disclosure is directed to a nucleic acid (e.g., DNA or RNA, including mRNA) encoding one or more peptides, polypeptides, concatemeric peptides, and/or chimeric or fusion polypeptides as described herein. For example, in aspects, the instant disclosure is directed to a nucleic acid encoding a peptide or polypeptide comprising, consisting of, or consisting essentially of one or more peptides or polypeptides having an amino acid sequence of SEQ ID NOS: 1-65 (and/or fragments or variants thereof), and optionally 1 to 12 additional amino acids distributed in any ratio on the N terminus and/or C-terminus of the polypeptide of SEQ ID NOS: 1-65. Additionally, in aspects, the instant disclosure is directed to a nucleic acid encoding a concatemeric polypeptide comprising, consisting of, or consisting essentially of one or more of SEQ ID NOS: 66-67 and 70-71. In aspects, a nucleic acid may have a sequence comprising, consisting of, or consisting essentially of one of more of SEQ ID NOS: 68, 69, 72, 73-77 (and fragments or variants thereof). In aspects, the present disclosure is directed to a plasmid having a sequence comprising, consisting of, or consisting essentially of one of more of SEQ ID NOS: 68, 69, 72, 73-77, or in aspects having a sequence comprising, consisting of, or consisting essentially of one of more of SEQ ID NOS: 69, 73, 75, and 77. In aspects, the present disclosure is directed to a vector, such as an expression vector, comprising such a nucleic acid as described herein. In aspects, the present disclosure is directed to a cell or vaccine comprising such a vector as described. In aspects, the present disclosure is directed to a cell comprising a vector of the present disclosure.

In aspects, the instant disclosure is directed to a vaccine comprising a T-cell epitope compound or composition of the instant disclosure (e.g., one or more of: polypeptides as disclosed herein; concatemeric peptides as disclosed herein; chimeric or fusion polypeptide compositions as disclosed herein; nucleic acids as disclosed herein, including nucleic acids encoding such peptides, polypeptides, concatemeric peptides, or chimeric of fusion polypeptide compositions as disclosed herein; expression cassettes, plasmids, expression vectors, recombinant viruses, cells as disclosed herein; pharmaceutical compositions as disclosed herein; or vaccines as described herein) and, optionally, a carrier, excipient, and/or an adjuvant.

In aspects, the instant disclosure is directed to a pharmaceutical composition, the pharmaceutical composition comprising a T-cell epitope compound or composition of the instant disclosure (e.g., one or more of: polypeptides as disclosed herein; concatemeric peptides as disclosed herein; chimeric or fusion polypeptide compositions as disclosed herein; nucleic acids as disclosed herein, including nucleic acids encoding such peptides, polypeptides, concatemeric peptides, or chimeric of fusion polypeptide compositions as disclosed herein; expression cassettes, plasmids, expression vectors, recombinant viruses, cells as disclosed herein) and a pharmaceutically acceptable carrier, excipient, and/or adjuvant. In aspects of the above-described pharmaceutical compositions, the composition comprises at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 30, at least 40, at least 50, at least 100, at least 200, at least 300, at least 400, at least 500, at least 600, at least 700, at least 800, at least 900, or at least 1000 peptides, polypeptides, or concatemeric peptides, as disclosed herein, including every value or range therebetween. In aspects, the present disclosure is directed to a pharmaceutical composition, the pharmaceutical composition comprising one or more nucleic acids as disclosed herein, including nucleic acids encoding one or more peptides, polypeptides, concatemeric peptides, and/or chimeric or fusion polypeptides as disclosed herein (as well as expression cassettes, plasmids, expression vectors, recombinant viruses, or cells as disclosed herein), and a pharmaceutically acceptable carrier, excipient, and/or an adjuvant.

In aspects, the present disclosure is directed to to methods of immunizing or inducing an immune response in a subject, comprising administering to said subject a T-cell epitope compound or composition of the instant disclosure (e.g., one or more of: polypeptides as disclosed herein; concatemeric peptides as disclosed herein; chimeric or fusion polypeptide compositions as disclosed herein; nucleic acids as disclosed herein, including nucleic acids encoding such peptides, polypeptides, concatemeric peptides, or chimeric or fusion polypeptide compositions as disclosed herein; expression cassettes, plasmids, expression vectors, recombinant viruses, cells as disclosed herein; pharmaceutical compositions as disclosed herein; or vaccines as described herein). In one aspect, the subject includes non-human animals (e.g., pigs). In aspects, the present disclosure is directed to a method of stimulating, inducing, and/or expanding an immune response to ASFV and/or associated diseases in a subject, comprising administering to said subject a T-cell epitope compound or composition of the instant disclosure. In one aspect, the subject includes non-human animals (e.g., pigs).

In aspects, the present disclosure is directed to methods of treating and/or preventing ASFV and/or associated diseases in a subject, such as a non-human animal (e.g., pigs), said methods comprising administering to said subject a T-cell epitope compound or composition of the instant disclosure (e.g., one or more of: polypeptides as disclosed herein; concatemeric peptides as disclosed herein; chimeric or fusion polypeptide compositions as disclosed herein; nucleic acids as disclosed herein, including nucleic acids encoding such peptides, polypeptides, concatemeric peptides, or chimeric or fusion polypeptide compositions as disclosed herein; expression cassettes, plasmids, expression vectors, recombinant viruses, cells as disclosed herein; pharmaceutical compositions as disclosed herein; or vaccines as described herein).

As should be understood, the T-cell epitope compounds or compositions of the instant disclosure may be used to induce an immune response and/or to vaccinate a subject. It is particularly useful to vaccinate swine against ASFV infection or ASF-diseases.

### BRIEF DESCRIPTION OF THE FIGURES

The present disclosure may be better understood with reference to the following figures. FIG. 1 is a schematic representation of an exemplary Class I plasmid construct. This exemplary Class I plasmid construct comprises 5067 base pairs containing DNA sequence SEQ. ID NO 69. High-purity plasmid for immunizations was prepared by Nature Technology Corporation, Inc. at research grade and underwent quality control testing. The plasmid DNA includes a genetic sequence expressing 40 putative class I epitopes and 16 AAY cleavage promoting motifs. Tandem stop codons were incorporated downstream of the epitope sequences. Class I genes were subcloned at predefined flanking restriction sites downstream of a destabilizing UbiquitinA76 tag (“UbA76”) in pNTC8684-eRNA41H for proteasome targeting.

FIG. 2 is a schematic representation of an exemplary Class II plasmid construct. This exemplary Class II plasmid construct comprises 5246 base pairs containing DNA sequence SEQ. ID NO 73. High-purity plasmid for immunizations was prepared by Nature Technology Corporation, Inc. at research grade and underwent quality control testing. The plasmid DNA includes a genetic sequence expressing 26 putative T-cell epitope clusters; breaker sequences were not required. Tandem stop codons were incorporated downstream of the epitope sequences. Class II genes were subcloned at predefined flanking restriction sites downstream of a tissue plasminogen activator (“TPA”) leader sequence in pNTC8682-eRNA-41H for secretory pathway targeting.

FIG. 3 is a schematic of an exemplary vaccine design strategy for developing an ASF DNA vaccine using the iVax (EpiVax, Providence, Rhode Island) toolkit.

FIG. 4 displays an experimental design for an ASF vaccine immunogenicity pilot study. Four-week old pigs were primed with plasmid DNA vaccine delivered intradermally using Pulse NeedleFree technology. Three weeks later, pigs were boosted with plasmid DNA vaccine delivered intradermally using Pulse NeedleFree technology. One week after the boost, blood was collected for an IFNy ELISpot assay.

FIG. 5 shows the results of an IFNy ELISpot assay. Four-week old pigs (N=3) were primed and boosted three weeks later with an ASF DNA vaccine designed using the iVax computational vaccine design platform. A matching group of pigs received empty plasmid (no epitopes) as a control. One week later, blood was collected, peripheral blood leukocytes isolated and recall responses to vaccine epitopes measured by IFNy ELISpot assay. Epitope-specific IFNy responses were detected in all pigs that received the ASF DNA vaccine, while no control pigs responded.

FIG. 6 displays an exemplary experimental design for an ASF vaccine immunogenicity pilot study. Four-week old pigs were primed with plasmid DNA vaccine delivered intradermally using Pulse NeedleFree technology. Three weeks after priming, pigs were boosted with plasmid DNA vaccine delivered intradermally using Pulse NeedleFree technology. One week after the boost, blood was collected for an IFNy ELISpot assay. Six weeks after priming, pigs were boosted with plasmid DNA and MCA adjuvant vaccine delivered intradermally using Pulse NeedleFree technology. One week later, blood was collected, peripheral blood leukocytes isolated and recall responses to vaccine epitopes measured by IFNγ ELISpot assay.

FIG. 7 depicts the plate set-up for an IFNy ELISpot assay following the first pDNA vaccine boost at three weeks after priming. There were 40 class I peptides and 26 class II peptides. The ELISpot plates included samples from 3 pigs per plate.

FIG. 8 depicts the plate set-up for an IFNy ELISpot assay following the second pDNA vaccine boost, in which the boost vaccine also included MCA adjuvant, at six weeks after priming. There were 40 class I peptides and 26 class II peptides. The ELISpot plates included samples from 3 pigs per plate.

FIGS. 9A-B show the results of an IFNγ ELISpot assay. Four-week old pigs (N=3) were primed and boosted three weeks later with an ASF DNA vaccine designed using the iVax computational vaccine design platform. A matching group of pigs received empty plasmid (no epitopes) as a control. One week later, blood was collected, peripheral blood leukocytes isolated and recall responses to vaccine epitopes measured by IFNy ELISpot assay. Epitope-specific IFNy responses were detected in all pigs that received the ASF DNA vaccine, while no control pigs responded.

FIGS. 10A-B shows the results of an IFNy ELISpot assay. Four-week old pigs (N=3) were primed and boosted three weeks and six weeks later with an ASF DNA vaccine designed using the iVax computational vaccine design platform. The six-week boost vaccine also included an MCA adjuvant. A matching group of pigs received empty plasmid (no epitopes) as a control. One week after the six-week boost, blood was collected, peripheral blood leukocytes isolated and recall responses to vaccine epitopes measured by IFNy ELISpot assay. Epitope-specific IFNy responses were detected in two out of the three pigs that received the ASF DNA vaccine, while no control pigs responded.

### DETAILED DESCRIPTION OF THE INVENTION

The present disclosure generally relates to T-cell epitope-based compounds and compositions useful for a vaccine against African Swine Fever Virus (“ASFV”). The T-cell epitope compounds and compositions of the instant disclosure include immunogenic polypeptides, and concatemeric peptides and the uses thereof, particularly in pharmaceutical and vaccine compositions. The disclosure also relates to nucleic acids, vectors, and cells which encode or express the peptides, polypeptides, concatemeric peptides, and chimeric or fusion polypeptides, and the uses thereof and the uses thereof. The polypeptides, and concatemeric peptides of the present disclosure more specifically comprise an amino acid sequence comprising an agretope predicted to be a ligand of SLA class I and/or SLA class II MHC molecules, as well as an epitope that is predicted to be recognized by effector CD8+ and CD4+ T-cells in the context of MHC class I and/or class II molecules, respectively. The instant disclosure is particularly suited to produce vaccines for non-human animals, particularly for vaccinating swine against ASFV infection.

It is possible to exploit epitopes specific T-cells to induce immunity against specific antigens. This discovery has implications for the design of therapeutic regimens and antigen-specific therapies against particular pathogens and infections. Administration of a drug, a protein, or an inactivated or live attenuated virus in conjunction with T-cell epitopes, including a T-cell epitope compound and composition of the present disclosure (including one or more of e.g., polypeptides having a sequence comprising, consisting of, or consisting essentially of one or more of SEQ ID NOS: 1-65 and/or fragments and variants thereof, and optionally 1 to 12 additional amino acids distributed in any ratio on the N terminus and/or C-terminus of the polypeptide of SEQ ID NOS:NOS: 1-65 and/or fragments and variants thereof as disclosed herein; concatemeric peptides as disclosed herein, including concatemeric polypeptides comprising, consisting of, or consisting essentially of one or more of SEQ ID NOS: 66-67 and 70-71 and/or fragments or variants thereof as disclosed herein; chimeric or fusion polypeptide compositions as disclosed herein; nucleic acids encoding such peptides, polypeptides, concatemeric peptides, or chimeric or fusion polypeptide compositions as disclosed herein, including nucleic acids having a sequence comprising, consisting of, or consisting essentially of one of more of SEQ ID NOS 68, 69, 72, 73-77 as disclosed herein; expression cassettes, plasmids, expression vectors, recombinant viruses, or cells which express such peptides, polypeptides, concatemeric peptides, chimeric or fusion polypeptide compositions, or nucleic acids as disclosed herein; vaccine compositions or formulations; and/or pharmaceutical compositions or formulations as disclosed herein) can induce immunity against a pathogen, including ASFV and related diseases. T-cell epitopes, including the T-cell epitope compounds and compositions of the present disclosure, can be used to deliberately manipulate the immune system toward immunity.

For example, the T-cell epitope compounds compositions of the present disclosure are useful in the selective engagement and activation of immunogenic T-cells. It is demonstrated herein that certain naturally occurring T-cells (in aspects, including CD4+ and CD8+ T-cells), can be engaged, activated, and/or applied to induce immunity or induce an immune response against pathogens such as ASFV and/or related diseases. By using the T-cell epitope compounds and compositions of the present disclosure to selectively activate naturally occurring T-cells, it is herein shown that such T-cell epitope compounds and compositions can be used to stimulate, induce, and/or expand an immune response to ASFV and/or associated diseases in a subject, and thus can be used in methods of treating and/or preventing ASFV and/or associated diseases in a subject, such as a non-human animal (e.g., pigs)t.

### Definitions

To further facilitate an understanding of the present invention, a number of terms and phrases are defined below. Unless otherwise defined, all terms (including technical and scientific terms) used herein have the same meaning as commonly understood by one of ordinary skill in the art to which this disclosure belongs. It will be further understood that terms such as those defined in commonly used dictionaries, should be interpreted as having a meaning that is consistent with their meaning in the context of the relevant art and the present disclosure, and will not be interpreted in an idealized or overly formal sense unless expressly so defined herein.

As used herein, the term “biological sample” as refers to any sample of tissue, cells, or secretions from an organism.

As used herein, the term “medical condition” includes, but is not limited to, any condition or disease manifested as one or more physical and/or psychological symptoms for which treatment and/or prevention is desirable, and includes previously and newly identified diseases and other disorders.

As used herein, the term “immune response” refers to the concerted action of lymphocytes, antigen presenting cells, phagocytic cells, granulocytes, and soluble macromolecules produced by the above cells or the liver (including antibodies, cytokines, and complement) that results in selective damage to, destruction of, or elimination from the human body of cancerous cells, metastatic tumor cells, malignant melanoma, invading pathogens, cells or tissues infected with pathogens, or, in cases of autoimmunity or pathological inflammation, normal human cells or tissues.

As used herein, the term “effective amount”, “therapeutically effective amount”, or the like of a composition, including a T-cell epitope compound or composition of the present disclosure (including one or more of e.g., polypeptides having a sequence comprising, consisting of, or consisting essentially of one or more of SEQ ID NOS: 1-65 and/or fragments and variants thereof, and optionally 1 to 12 additional amino acids distributed in any ratio on the N terminus and/or C-terminus of the polypeptide of SEQ ID NOS:NOS: 1-65 as disclosed herein; concatemeric peptides as disclosed herein, including concatemeric polypeptides comprising, consisting of, or consisting essentially of one or more of SEQ ID NOS: 66-67 and 70-71 and/or fragments or variants thereof as disclosed herein; chimeric or fusion polypeptide compositions as disclosed herein; nucleic acids encoding such peptides, polypeptides, concatemeric peptides, or chimeric or fusion polypeptide compositions as disclosed herein, including nucleic acids having a sequence comprising, consisting of, or consisting essentially of one of more of SEQ ID NOS 68, 69, 72, 73-77 and/or fragments or variants thereof as disclosed herein; expression cassettes, plasmids, expression vectors, recombinant viruses, or cells which express such peptides, polypeptides, concatemeric peptides, chimeric of fusion polypeptide compositions, or nucleic acids as disclosed herein; vaccine compositions or formulations as disclosed herein; and/or pharmaceutical compositions or formulations as disclosed herein) is a quantity sufficient to achieve a desired therapeutic and/or prophylactic effect, e.g., an amount that results in the prevention of, or a decrease in, the symptoms associated with a disease that is being treated, such as ASFV and/or related diseases. The amount of a composition of the present disclosure administered to the subject (e.g., a pig) will depend on the type and severity of the disease and on the characteristics of the individual, such as general health, age, sex, body weight and/or tolerance to drugs. It will also depend on the degree, severity and type of pathogen and/or disease. The skilled artisan will be able to determine appropriate dosages depending on these and other factors. The compositions of the present invention can also be administered in combination with each other or with one or more additional therapeutic compounds.

As used herein, “anti-ASFV activity”, “anti-ASFV polypeptides”, “anti-ASFV compositions”, and the like are intended to mean that the T-cell epitope compounds and compositions of the of the present diclsoure (including polypeptides, concatemeric polypeptides, chimeric or fusion proteins, nucleic acids, plasmids, vectors, pharmaceutical compositions, vaccines, and other compositions of the instant disclosure) have anti-ASFV activity and thus are capable of suppressing, controlling, and/or killing an invading ASF virus. For example, anti-ASFV activity means that the instantly-disclosed therapeutic T-cell epitope comopounds and compositions are, in aspects: capable of stimulating, inducing, and/or expanding an immune response to ASFV (e.g., a cellular or humoral immune response to ASFV) and/or associated diseases in a subject; capable of stimulating, inducing, and/or expanding an ASFV-specific IFNy response (e.g., by lymphocytes such as PMBC, or effector CD4+ and/or CD8+ T-cells), and/or capable of inducing immunity against ASFV. In aspects, a T-cell epitope compound or composition of the present disclosure having anti-ASFV activity will reduce the disease symptoms resulting from ASFV challenge by at least about 5% to about 50%, at least about 10% to about 60%, at least about 30% to about 70%, at least about 40% to about 80%, or at least about 50% to about 90% or greater, including any value or range therebetween. Anti-ASFV activity can be determined by various experiments and assays as known to those of skill in the art, including methods such as by antibody titrations of sera, e.g., by ELISA and/or seroneutralization assay analysis and/or by vaccination challenge evaluation, including the experiments and assays as disclosed in the Examples herein.

As used herein, the term “T-cell epitope” means an MHC ligand or protein determinant, 7 to 30 amino acids in length, and capable of specific binding to swine leukocyte antigen (SLA) molecules and interacting with specific T-cell receptors (TCRs). Generally, T-cell epitopes are linear and do not express specific three-dimensional characteristics. T-cell epitopes are not affected by the presence of denaturing solvents. The ability to interact with T-cell epitopes can be predicted by in silico methods (De Groot AS et al., (1997), AIDS Res Hum Retroviruses, 13(7):539-41; Schafer JR et al., (1998), Vaccine, 16(19): 1880-4; De Groot AS et al., (2001), Vaccine, 19(31):4385-95; De Groot AR et al.,(2003), Vaccine, 21(27-30):4486-504, all of which are herein incorporated by reference in their entirety. The term “immune-stimulating T-cell epitope polypeptide” refers to a molecule capable of inducing an immune response, e.g., a humoral, T-cell-based, or innate immune response.

As used herein, the term “T-cell epitope cluster” refers to polypeptide that contains between about 4 to about 40 MHC binding motifs. In particular embodiments, the T-cell epitope cluster contains between about 5 to about 35 MHC binding motifs, between about 8 and about 30 MHC binding motifs; and between about 10 and 20 MHC binding motifs.

As used herein, the term “immune-stimulating T-cell epitope polypeptide” refers to a molecule capable of inducing an immune response, e.g., a humoral, T-cell-based, or innate immune response.

As used herein, the term “B-cell epitope” means a protein determinant capable of specific binding to an antibody. B-cell epitopes usually consist of chemically active surface groupings of molecules such as amino acids or sugar side chains and usually have specific three-dimensional structural characteristics, as well as specific charge characteristics. Conformational and non-conformational epitopes are distinguished in that the binding to the former but not the latter is lost in the presence of denaturing solvents.

The term “subject” as used herein refers to any living organism in which an immune response is elicited. The term subject includes, but is not limited to, humans, nonhuman primates such as chimpanzees and other apes and monkey species; farm animals such as cattle, sheep, pigs, goats and horses; domestic mammals such as dogs and cats; laboratory animals including rodents such as mice, rats and guinea pigs, and the like. The term does not denote a particular age or sex. Thus, adult and newborn subjects, as well as fetuses, whether male or female, are intended to be covered.

As used herein, the term “MHC complex” refers to a protein complex capable of binding with a specific repertoire of polypeptides known as SLA ligands and transporting said ligands to the cell surface.

As used herein, the term “MHC Ligand” means a polypeptide capable of binding to one or more specific MHC alleles. The term “SLA ligand” is interchangeable with the term “MHC Ligand”.

As used herein, the term “T-cell Receptor” or “TCR” refers to a protein complex expressed by T-cells that is capable of engaging a specific repertoire of MHC/Ligand complexes as presented on the surface of cells.

As used herein, the term “MHC Binding Motif” refers to a pattern of amino acids in a protein sequence that predicts binding to a particular MHC allele.

As used herein, the term “AAY cleavage motif” refers to the short amino acid motif consisting of the sequence “alanine-alanine-tyrosine” capable of promoting proteasome-mediated cleavage of a peptide or protein, promoting the binding of the transporter associated with antigen processing to a peptide or protein, and/or increasing proteasome degradation at specific sites within a peptide or protein.

As used herein, the term “Immune Synapse” means the protein complex formed by the simultaneous engagement of a given T-cell epitope to both a cell surface MHC complex and TCR.

The term “polypeptide” refers to a polymer of amino acids, and not to a specific length; thus, peptides, oligopeptides and proteins are included within the definition of a polypeptide. As used herein, a polypeptide is said to be “isolated” or “purified” when it is substantially free of cellular material when it is isolated from recombinant and non-recombinant T-cells, or free of chemical precursors or other chemicals when it is chemically synthesized. A polypeptide (e.g., a polypeptide comprising, consisting of, or consisting essentially of one or more of SEQ ID NOS: 1-67, 70-71 or variants and fragments thereof, which in aspects may be isolated, synthetic, and/or recombinant) of the present disclosure, however, can be joined to, linked to, or inserted into another polypeptide (e.g., a heterologous polypeptide) with which it is not normally associated in a cell and still be “isolated” or “purified.” Additionally, one or more T-cell epitopes of the present disclosure can be joined to, linked to, or inserted into another polypeptide wherein said one or more T-cell epitopes of the present disclosure is not naturally included in the polypeptide and/or said one or more T-cell epitopes of the present disclosure is not located at its natural position in the polypeptide. When a polypeptide is recombinantly produced, it can also be substantially free of culture medium, for example, culture medium represents less than about 20%, less than about 10%, or less than about 5% of the volume of the polypeptide preparation.

As used herein, a “concatemeric” peptide or polypeptide refers to a series of at least two peptides or polypeptides linked together. Such linkages may form a string-of-beads design. In aspects, concatemeric polypeptides of the instant disclosure include concatemeric polypeptides comprising, consisting of, or consisting essentially of one or more of SEQ ID NOS: 66-67 and 70-71, and/or fragments or variants thereof (which in aspects may be isolated, synthetic, and/or recombinant).

As used herein, the term “purpose built computer program” refers to a computer program designed to fulfill a specific purpose; typically to analyze a specific set of raw data and answer a specific scientific question.

As used herein, the term “z-score” indicates how many standard deviations an element is from the mean. A z-score can be calculated from the following formula: z = (X - µ) / σ ; where z is the z-score, X is the value of the element, µ is the population mean, and σ is the standard deviation.

As used herein, the term “stimulation index” (which may alternatively be termed “SI”) is used in reference to an ELISpot assay as the ratio of the mean spot number in the presence of test peptide to the mean spot number in the presence of peptide diluent. In ELISpot assays, the stimulation index allows comparison between subjects receiving a vaccine and control subjects while taking account of background, such as spontaneous responsiveness.

As used herein, the singular forms “a,” “an,” and “the” are intended to include the plural forms, including “at least one,” unless the content clearly indicates otherwise. “Or” means “and/or.” As used herein, the term “and/or” and “one or more” includes any and all combinations of the associated listed items. For example, the term “one or more” with respect to the “one or more of SEQ ID NOS: 1-65 of the present disclosure” includes any and all combinations of SEQ ID NOS: 1-65. The term “or a combination thereof” means a combination including at least one of the foregoing elements.

The following abbreviations and/or acronyms are used throughout this application:
ASF African swine fever
ASFV African swine fever virus
AAY Alanine-Alanine-Tyrosine cleavage motif
EDTA ethylenediaminetetraacetic acid
IFNγ interferon gamma
MCA mannosylated chitosan
MHC major histocompatibility complex
PBMC peripheral blood mononuclear cell
pDNA plasmid DNA
PWC pokeweed mitogen
SFC spot-forming cell
SLA swine leukocyte antigen
TCR T-cell receptor
w/w weight by weight

A “variant” polypeptide (including a variant T-cell epitope) can differ in amino acid sequence by one or more substitutions, deletions, insertions, inversions, fusions, and truncations or a combination of any of these. In aspects, a variant T-cell epitope can differ in amino acid sequence by one or more substitutions, deletions, insertions, inversions, fusions, and truncations or a combination of any of these provided said variants retain MHC binding propensity and/or TCR specificity.

The present disclosure also includes fragments of the peptide or polypeptides of the instant disclosure. In aspects, the disclosure also encompasses fragments of the variants of the T-cell epitopes described herein, provided said fragments and/or variants at least in part retain MHC binding propensity and/or TCR specificity, or retain anti-ASFV activity.

The present disclosure also provides chimeric or fusion polypeptides (which in aspects may be isolated, synthetic, and/or recombinant) wherein one or more of the instantly-disclosed peptides, polypeptides, or concatemeric peptides is a part thereof. In aspects, a chimeric or fusion polypeptide composition comprises one or more polypeptides of the instant disclosure linked to a heterologous polypeptide. As previously stated, the term “heterologous polypeptide” is intended to mean that the one or more T-cell epitopes (e.g., one or more of SEQ ID NOS: 1-67, 70-71) are heterologous to, or not included naturally, in the heterologous polypeptide. In aspects, the one or more peptides, polypeptides, or concatemeric peptides of the instant disclosure may be inserted into the heterologous polypeptide (e.g., through mutagenesis or other known means in the art), may be added to the C-terminus (with or without the use of linkers, as is known in the art), and/or added to the N-terminus (with or without the use of linkers, as is known in the art) of the heterologous polypeptide. For example, protein engineering by mutagenesis can be performed using site-directed mutagenesis techniques, or other mutagenesis techniques known in the art (see e.g., James A. Brannigan and Anthony J. Wilkinson., 2002, Protein engineering 20 years on. Nature Reviews Molecular Cell Biology 3, 964-970; Turanli-Yildiz B. et al., 2012, Protein Engineering Methods and Applications, intechopen.com, which are herein incorporated by reference in their entirety).

In aspects, chimeric or fusion polypeptides comprise one or more peptides, polypeptides, or concatemeric peptides of the instant disclosure operatively linked to a heterologous polypeptide. “Operatively linked” indicates that the polypeptide (e.g., the one or more T-cell epitope polypeptides of the present disclosure) and the heterologous protein are fused in-frame or chemically-linked or otherwise bound. In aspects, an isolated, synthetic, and/or recombinant chimeric or fusion polypeptide composition comprises a polypeptide, said polypeptide having a sequence comprising one or more of SEQ ID NOS: 1-67, 70-71 of the present disclosure, wherein said one or more of SEQ ID NOS: 1-67, 70-71 is not naturally included in the polypeptide and/or said of one or more of SEQ ID NOS: 1-67, 70-71 is not located at its natural position in the polypeptide. In aspects, the one or more of SEQ ID NOS: 1-67, 70-71 of the present disclosure can be joined, linked to (e.g., fused in-frame, chemically-linked, or otherwise bound), and/or inserted into the polypeptide. In aspects, the one or more of SEQ ID NOS: 1-67, 70-71 of the present disclosure can be joined or linked to (e.g., fused in-frame, chemically-linked, or otherwise bound) to a small molecule, drug, or drag fragment, for example but not limited to, a drug or drug fragment that is binds with high affinity to defined SLAs. In aspects of the above chimeric or fusion polypeptide compositions, the one or more polypeptides (e.g., T-cell epitope polypeptides) of the present disclosure have a sequence comprising, consisting of, or consisting essentially of one or more of SEQ ID NOS: 1-65. In aspects, the instantly-disclosed chimeric or fusion polypeptides may be isolated, synthetic, and/or recombinant.

An “isolated” peptide, polypeptide, concatemeric peptide (e.g., an isolated T-cell activating T-cell epitope or T-cell epitope polypeptide), chimeric or fusion polypeptide, or nucleic acid can be purified from cells that naturally express it, purified from cells that have been altered to express it (recombinant), or synthesized using known synthesis methods. In one embodiment, a T-cell epitope compound or composition of the instant disclosure is produced by recombinant DNA or RNA techniques. For example, a nucleic acid molecule encoding the peptide, polypeptide, concatemeric peptide, or chimeric or fusion polypeptide is cloned into an expression vector, the expression vector introduced into a host cell, and the polypeptide expressed in the host cell. The peptide, polypeptide, concatemeric peptide, or chimeric or fusion polypeptide can then be isolated from the cells by an appropriate purification scheme using standard protein purification techniques.

For the purposes of the present disclosure, peptides, polypeptides, concatemeric peptides, or chimeric or fusion polypeptides of the instant disclosure can include, for example, modified forms of naturally occurring amino acids such as D-stereoisomers, non-naturally occurring amino acids; amino acid analogs; and mimetics. Further, in aspects, peptides, polypeptides, concatemeric peptides, or chimeric or fusion polypeptides of the instant disclosure can include retro-inverso peptides of the instantly disclosed peptides, polypeptides, concatemeric peptides, or chimeric or fusion polypeptides of the instant disclosure, provided said peptides, polypeptides, concatemeric peptides, or chimeric or fusion polypeptides of the instant disclosure at least in part retain MHC binding propensity and/or TCR specificity, and/or retain ASFV activity.

Although any methods and materials similar or equivalent to those described herein can be used in the practice or testing of the present disclosure, the preferred methods and materials are described. Other features, objects, and advantages of the present disclosure will be apparent from the description and the Claims. In the Specification and the appended Claims, the singular forms include plural referents unless the context clearly dictates otherwise. Unless defined otherwise, all technical and scientific terms used herein have the same meaning as commonly understood by one of ordinary skill in the art to which this disclosure belongs. All references cited herein are incorporated herein by reference in their entirety and for all purposes to the same extent as if each individual publication, patent, or patent application was specifically and individually indicated to be incorporated by reference in its entirety for all purposes.

### Polypeptides, Concatemeric Polypeptides, and Chimeric or Fusion Polypeptides

In aspects, the T-cell epitope compounds and compositions of the instant diclosure include one or more of the following T-cell epitope polypeptides (as well as fragments thereof, variants thereof, and fragments of such variants, provided said fragments and/or variants retain functionality, including MHC binding propensity and/or TCR specificity, or anti-ASFV activity):
For SLA Class I: SEQ. ID NOS 1-39
For SLA Class II: SEQ. ID NOS 40-65

In aspects, the present disclosure provides a novel class of T-cell epitopes (which may be isolated, synthetic, and/or recombinant), which comprise a peptide or polypeptide chain derived from common swine proteins, and in aspects from ASFV proteins (e.g., encoded proteins from the ASFV genome). T-cell epitopes of the present disclosure are highly conserved among known variants of their source proteins (e.g., present in more than 10% of known variants). Such conserved T-cell epitopes were first identified by the Conservatrix system as described in Examples 1 and 2. Complete proteomes of 21 ASFV strains were downloaded from GenBank and proteins were loaded to iVAX and parsed into 9-mers overlapping by eight amino acids using the Conservatrix algorithm. The Conservatrix system (EpiVax, Providence, Rhode Island) is an algorithm useful for identifying 9-mer polypeptide sequences from a larger set of data. The Conservatrix system parses input sequences into 9-mer sequences that are conserved amongst multiple inputted whole sequences, such as multiple strains of the same pathogen, for even the most mutable of potential vaccine targets. These 9-mer sequences may be searched for identically matched 9-mer sequences across data sets. Nine-mer sequences were searched for identically matched 9-mers among ASFV strains.

As also described in Examples 1 and 2, T-cell epitopes of the present disclosure comprise at least one putative T-cell epitope as identified by PigMatrix™ analysis. PigMatrix™ is a proprietary computer algorithm developed by EpiVax (Providence, Rhode Island), which is used to screen protein sequences for the presence of putative T-cell epitopes. Input sequences are, for example, parsed into overlapping 9-mer frames where each frame overlaps the last by 8 amino acids. Each of the resulting frames is then scored for predicted binding affinity with respect to a panel of eight common Class I SLA alleles (SLA-I*0101, 1*0401, 1*0801, 1*1201, 1*1301, 2*0101, 2*0401, 2*0501, 2*1201, 3*0401, 3*0501, 3*0601, 3*0701) and Class II SLA alleles (SLA-DRB1*0101, 0201, 0401, 0402, 0601, 0602, 0701, 1001). Raw scores are normalized against the scores of a large sample of randomly generated peptides. The resulting “Z” score is reported. In aspects, any 9-mer peptide with an allele-specific PigMatrix™ Z-score in excess of 1.64, theoretically the top 5% of any given sample, is considered a putative T-cell epitope.

Peptides containing clusters of putative T-cell epitopes are more likely to test positive in validating in vitro and in vivo assays. In aspects, the results of the initial PigMatrix™ analysis are further screened for the presence of putative T-cell epitope “clusters” using a second proprietary algorithm known as Clustimer™ algorithm. The Clustimer™ algorithm identifies sub-regions contained within any given amino acid sequence that contains a statistically unusually high number of putative T-cell epitopes. Typical T-cell epitope “clusters” range from about 9 to roughly 30 amino acids in length and, considering their affinity to multiple alleles and across multiple 9-mer frames, can contain anywhere from about 4 to about 40 putative T-cell epitopes. Each epitope cluster identified an aggregate PigMatrix™ score is calculated by summing the scores of the putative T-cell epitopes and subtracting a correcting factor based on the length of the candidate epitope cluster and the expected score of a randomly generated cluster of the same length. PigMatrix™ cluster scores in excess of +10 are considered significant. In aspects, the T-cell epitopes of the instant disclosure contain several putative T-cell epitopes forming a pattern known as a T-cell epitope cluster.

Putative T-cell epitopes were also screened for cross-conservation with the pig proteome using JanusMatrix, as further described in Examples 1 and 2. The JanusMatrix system (EpiVax, Providence, Rhode Island) is useful for screening peptide sequences for cross-conservation with a host proteome. JanusMatrix is an algorithm that predicts the potential for cross-reactivity between peptide clusters and the host genome or proteome, based on conservation of TCR-facing residues in their putative MHC ligands. The JanusMatrix algorithm first considers all the predicted epitopes contained within a given protein sequence and divides each predicted epitope into its constituent agretope and epitope. Each sequence is then screened against a database of host proteins. Peptides with a compatible MHC-facing agretope (i.e., the agretopes of both the input peptide and its host counterparty are predicted to bind the same MHC allele) and exactly the same TCR-facing epitope are returned. The JanusMatrix Homology Score suggests a bias towards immune tolerance. In the case of a vaccine, cross-conservation between host (e.g., pig) epitopes and the antigenic epitopes may indicate that such a candidate utilizes immune camouflage, thereby evading the immune response and making for an ineffective vaccine. When the host is, for example, a pig, the peptide clusters are screened against pig genomes and proteomes, based on conservation of TCR-facing residues in their putative HLA ligands. The peptides are then scored using the JanusMatrix Swine Homology Score. In aspects, peptides with a JanusMatrix Swine Homology Score below 2.5 or below 2.0 indicate low tolerogenicity potential and may be useful for vaccines. In aspects, epitopes with JanusMatrix Swine Homology Scores below 2.5 (low tolerogenicity potential), or below 2.0, were selected.

In aspects, T-cell epitopes of the present disclosure bind to at least one and preferably two or more common SLA class I and/or class II alleles with at least a moderate affinity (e.g., in aspects, <1000 µM IC₅₀, <500 µM IC₅₀, <400 µM IC₅₀, <300 µM IC₅₀, or <200 µM IC₅₀ in SLA binding assays based on soluble SLA molecules). In aspects, T-cell epitopes of the present disclosure are capable of being presented at the cell surface by cells in the context of at least one and, in other aspects, two or more alleles of the SLA. In this context, the epitope-SLA complex can be recognized by naturally occurring T-cells having TCRs that are specific for the epitope-SLA complex and circulating in normal control subjects. In aspects, the recognition of the epitope-SLA complex can cause the matching T-cell to be activated and to secrete activating cytokines (e.g., effector cytokines such as IFNγ) and chemokines.

In aspects, a epitopic composition includes one or more peptides or polypeptides a disclosed herein. In aspects, the present disclosure is directed to a polypeptide having a sequence comprising, consisting of, or consisting essentially of one or more of SEQ ID NOS: 1-65, or fragments and variants thereof. The phrase “consisting essentially of” is intended to mean that a polypeptide according to the present disclosure, in addition to having the sequence according to any of SEQ ID NOS: 1-65 or a fragment or a variant thereof, contains additional amino acids or residues that may be present at either terminus of the peptide and/or on a side chain that are not necessarily forming part of the peptide that functions as an MHC ligand and provided they do not substantially impair the activity of the peptide to function as a T-cell epitope. In aspects, the peptides or polypeptides of the instant disclosure can be isolated, recombinant, and/or synthetic. In aspects, the peptides or polypeptides of the instant disclosure can be either in neutral (uncharged) or salt forms, and may be either free of or include modifications such as glycosylation, side chain oxidation, or phosphorylation. In aspects, the peptides or polypeptides of the instant disclosure can be capped with an n-terminal acetyl and/or c-terminal amino group.

In aspects, the instant disclosure is directed to a peptide or polypeptide comprising, consisting, or consisting essentially of an amino acid sequence of SEQ ID NOS: 1-65 (and/or fragments or variants thereof), and optionally 1 to 12 additional amino acids distributed in any ratio on the N terminus and/or C-terminus of the polypeptide of SEQ ID NOS: 1-65. In aspects, the instant disclosure is directed to a peptide or polypeptide have a core amino acid sequence comprising, consisting of, or consisting essentially of one or more peptides or polypeptides having an amino acid sequence of SEQ ID NOS: 1-65, and optionally having extensions of 1 to 12 amino acids on the C-terminal and/or the N-terminal of the core amino acid sequence, wherein the overall number of these flanking amino acids is 1 to 12, 1 to 3, 2 to 4, 3 to 6, 1 to 10, 1 to 8, 1 to 6, 2 to 12, 2 to 10, 2 to 8, 2 to 6, 3 to 12, 3 to 10, 3 to 8, 3 to 6, 4 to 12, 4 to 10, 4 to 8, 4 to 6, 5 to 12, 5 to 10, 5 to 8, 5 to 6, 6 to 12, 6 to 10, 6 to 8, 7 to 12, 7 to 10, 7 to 8, 8 to 12, 8 to 10, 9 to 12, 9 to 10, or 10 to 12, wherein the flanking amino acids can be distributed in any ratio to the C-terminus and the N-terminus (for example all flanking amino acids can be added to one terminus, or the amino acids can be added equally to both termini or in any other ratio). In aspects, the instant disclosure is directed to a peptide or polypeptide have a core sequence comprising, consisting of, or consisting essentially of one or more peptides or polypeptides having an amino acid sequence of SEQ ID NOS: 1-65 (and/or fragments and variants thereof), optionally with extensions of 1 to 12 amino acids on the C-terminal and/or the N-terminal, wherein the overall number of these flanking amino acids is 1 to 12, 1 to 3, 2 to 4, 3 to 6, 1 to 10, 1 to 8, 1 to 6, 2 to 12, 2 to 10, 2 to 8, 2 to 6, 3 to 12, 3 to 10, 3 to 8, 3 to 6, 4 to 12, 4 to 10, 4 to 8, 4 to 6, 5 to 12, 5 to 10, 5 to 8, 5 to 6, 6 to 12, 6 to 10, 6 to 8, 7 to 12, 7 to 10, 7 to 8, 8 to 12, 8 to 10, 9 to 12, 9 to 10, or 10 to 12, wherein the flanking amino acids can be distributed in any ratio to the C-terminus and the N-terminus (for example all flanking amino acids can be added to one terminus, or the amino acids can be added equally to both termini or in any other ratio), provided that the polypeptide with the flanking amino acids is still able to bind to a same HLA molecule (i.e., retain MHC binding propensity) as said polypeptide core sequence without said flanking amino acids. In aspects, said polypeptide with the flanking amino acids is still able to bind to a same HLA molecule (i.e., retain MHC binding propensity) and/or retain the same TCR specificity, and/or retain ASFV activity, as said polypeptide core sequence without said flanking amino acids. In aspects, said flanking amino acid sequences are those that also flank the peptides or polypeptides included therein in the naturally occurring protein. For example, for a peptide or polypeptide have a core sequence comprising, consisting of, or consisting essentially of one or more peptides or polypeptides having an amino acid sequence of SEQ ID NOS: 1-65 (and/or fragments and variants thereof), optionally with extensions of 1 to 12 amino acids on the C-terminal and/or the N-terminal, the extensions of 1 to 12 amino acids are those found flanking the amino acid sequence of SEQ ID NOS: 1-65 in the encoded protein sequence of African swine fever virus (ASFV), e.g., such as the extensions found flanking the amino acid sequences of SEQ ID NOS: 1-65 from one or more of the proteomes from the set of ASFV strains in Table 1 of Example 1. In aspects, said flanking amino acid sequences as described herein may serve as a MHC stabilizing region. The use of a longer peptide may allow endogenous processing by patient cells and may lead to more effective antigen presentation and induction of T-cell responses. In aspects, the peptides or polypeptides of the instant disclosure can be isolated, recombinant, and/or synthetic. In aspects, the peptides or polypeptides can be either in neutral (uncharged) or salt forms, and may be either free of or include modifications such as glycosylation, side chain oxidation, or phosphorylation. In certain aspects, the peptides or polypeptides of the instant disclosure can be capped with an n-terminal acetyl and/or c-terminal amino group.

In aspects, the instant disclosure is directed to a polypeptide comprising an amino acid sequence having at least 75%, 80%, 85%, 90%, or 95% homology to any one of SEQ ID NOS: 1-65, wherein said polypeptide retains MHC binding propensity and the same TCR specificity, and/or retains ASFV activity.

In aspects, the present disclosure is directed to a concatemeric polypeptide that comprises at one or more of the instantly-disclosed polypeptides or peptides (e.g., but not limited to, a peptide or polypeptide comprising, consisting, or consisting essentially of an amino acid sequence of SEQ ID NOS: 1-65 (and/or fragments or variants thereof), and optionally 1 to 12 additional amino acids distributed in any ratio on the N terminus and/or C-terminus of the polypeptide of SEQ ID NOS: 1-65) linked, fused, or joined together (e.g., fused in-frame, chemically-linked, or otherwise bound) to an additional peptide or polypeptide. Such additional peptide or polypeptide may be one or more of the instantly instantly-disclosed polypeptides or peptides, or may be an additional peptide or polypeptide of interest. In aspects a concatemeric peptide is composed of 3 or more, 4 or more, 5 or more, 6 or more, 7 or more, 8 or more, or 9 or more of the instantly-disclosed peptides or polypeptides. In other aspects, the concatemeric peptides or polypeptides include 1000 or more, 1000 or less, 900 or less, 500 or less, 100 or less, 75 or less, 50 or less, 40 or less, 30 or less, 20 or less, or 100 or less peptide epitopes. In yet other embodiments, a concatemeric peptide has 3-100, 5-100, 10-100, 15-100, 20-100, 25-100, 30-100, 35-100, 40-100, 45-100, 50-100, 55-100, 60-100, 65-100, 70-100, 75-100, 80-100, 90-100, 5-50, 10-50, 15-50, 20-50, 25-50, 30-50, 35-50, 40-50, 45-50, 100-150, 100-200, 100-300, 100-400, 100-500, 50-500, 50-800, 50-1,000, or 100-1,000 of the instantly-disclosed peptides or polypeptides linked, fused, or joined together. Each peptide or polypeptide of the concatemeric polypeptide may optionally have one or more linkers, which may optionally be cleavage-sensitive sites, adjacent to their N and/or C terminal end. In such a concatemeric peptide, two or more of the peptide epitopes may have a cleavage-sensitive site between them. Alternatively two or more of the peptide epitopes may be connected directly to one another or through a linker that is not a cleavage-sensitive site.

In aspects, a concatemeric polypeptide of the instant disclosure is produced using the EpiAssembler System (EpiVax). The EpiAssembler system is useful for assembling overlapping epitopes to Immunogenic Consensus Sequences (ICS). EpiAssembler is an algorithm that optimizes the balance between pathogen and population coverage. EpiAssembler uses the information from the sequences produced by Conservatrix and EpiMatrix to form highly immunogenic consensus sequences. In aspects, a concatemeric polypeptide comprises, consists of, or consists essentially of one or more of SEQ ID NOS: 66-67 and 70-71, and fragments or variants thereof, (which in aspects may be isolated, synthetic, and/or recombinant). In aspects, the present disclosure provides a polypeptide with at least 60%, 70%, 80%, 90%, or 95% homology to SEQ ID NO 66. In aspects, the present disclosure provides a polypeptide having anti-ASFV activity, said polypeptide with at least 60%, 70%, 80%, 90%, or 95% homology to SEQ ID NO 66. In aspects, the present disclosure provides a polypeptide with at least 60%, 70%, 80%, 90%, or 95% homology to SEQ ID NO 67. In aspects, the present disclosure provides a polypeptide having anti-ASFV activity, said polypeptide with at least 60%, 70%, 80%, 90%, or 95% homology to SEQ ID NO 67. In aspects, the present disclosure provides a polypeptide with at least 60%, 70%, 80%, 90%, or 95% homology to SEQ ID NO 70. In aspects, the present disclosure provides a polypeptide having anti-ASFV activity, said polypeptide with at least 60%, 70%, 80%, 90%, or 95% homology to SEQ ID NO 70. In aspects, the present disclosure provides a polypeptide with at least 60%, 70%, 80%, 90%, or 95% homology to SEQ ID NO 71. In aspects, the present disclosure provides a polypeptide having anti-ASFV activity, said polypeptide with at least 60%, 70%, 80%, 90%, or 95% homology to SEQ ID NO 71. As described previously, anti-ASFV activity includes, in aspects: being capable of stimulating, inducing, and/or expanding an immune response to ASFV (e.g., a cellular or humoral immune response to ASFV) and/or associated diseases in a subject; being capable of stimulating, inducing, and/or expanding an ASFV-specific IFNy response (e.g., by lymphocytes such as PMBC, or effector CD4+ and/or CD8+ T-cells), and/or being capable of inducing immunity against ASFV in a host subject (e.g., a pig). In aspects, such polypeptides having anti-ASFV activity will reduce the disease symptoms resulting from ASFV challenge by at least about 5% to about 50%, at least about 10% to about 60%, at least about 30% to about 70%, at least about 40% to about 80%, or at least about 50% to about 90% or greater, including any value or range therebetween. Again, anti-ASFV activity can be determined by various experimetns and assays as known to those of skill in the art, including the experiments and assays as disclosed in the Example section herein.

In aspects, the concatemeric polypeptides of the instant disclosure can be isolated, recombinant, and/or synthetic.In aspects, the concatemeric polypeptides can be either in neutral (uncharged) or salt forms, and may be either free of or include modifications such as glycosylation, side chain oxidation, or phosphorylation. In certain aspects, the concatemeric polypeptides of the instant disclosure can be capped with an n-terminal acetyl and/or c-terminal amino group.

In aspects, one or more peptides or polypeptides of the instant disclosure (e.g., but not limited to, a peptide or polypeptide comprising, consisting, or consisting essentially of an amino acid sequence of SEQ ID NOS: 1-67, 70-71 (and/or fragments or variants thereof)) may be joined to, linked to (e.g., fused in-frame, chemically-linked, or otherwise bound), and/or inserted into a heterologous polypeptide. As previously described, with respect to the one or more peptides or polypeptides of the instant disclosure, the term “heterologous polypeptide” is intended to mean that the one or more peptides or polypeptides of the instant disclosure are heterologous to, or not included naturally, in the heterologous polypeptide. In aspects, one or more of the instantly-disclosed polypeptides may be inserted into the heterologous polypeptide (e.g., through recombinant techniques, mutagenesis, or other known means in the art), may be added to the C-terminus (with or without the use of linkers, as is known in the art), and/or added to the N-terminus (with or without the use of linkers, as is known in the art) of the heterologous polypeptide. For example, protein engineering by mutagenesis can be performed using site-directed mutagenesis techniques, or other mutagenesis techniques known in the art (see e.g., James A. Brannigan and Anthony J. Wilkinson., 2002, Protein engineering 20 years on. Nature Reviews Molecular Cell Biology 3, 964-970; Turanli-Yildiz B. et al., 2012, Protein Engineering Methods and Applications, intechopen.com, which are herein incorporated by reference in their entirety). In aspects, the one or more peptides or polypeptides of the instant disclosure (e.g., but not limited to, a peptide or polypeptide comprising, consisting, or consisting essentially of an amino acid sequence of SEQ ID NOS: 1-67, 70-71 (and/or fragments or variants thereof)) may be joined to, linked to (e.g., fused in-frame, chemically-linked, or otherwise bound), and/or inserted into a heterologous polypeptide as a whole, although it may be made up from a joined to, linked to (e.g., fused in-frame, chemically-linked, or otherwise bound), and/or inserted amino acid sequence, together with flanking amino acids of the heterologous polypeptide. In aspects, the present disclosure is directed to polypeptide (which, in aspects, may be an isolated, synthetic, and/or recombinant) having a sequence comprising one or more of SEQ ID NOS: 1-67, 70-71 (and/or fragments or variants thereof), wherein said one or more of SEQ ID NOS: 1-67, 70-71 is not naturally included in the polypeptide and/or said one or more of SEQ ID NOS: 1-67, 70-71 is not located at its natural position in the polypeptide.

As used herein, two polypeptides (or a region of the polypeptides) are substantially homologous or identical when the amino acid sequences are at least about 45-55%, typically at least about 70-75%, more typically at least about 80-85%, more typically greater than about 90%, and more typically greater than 95% or more homologous or identical. To determine the percent homology or identity of two amino acid sequences, or of two nucleic acid sequences, the sequences are aligned for optimal comparison purposes (e.g., gaps can be introduced in the sequence of one polypeptide or nucleic acid molecule for optimal alignment with the other polypeptide or nucleic acid molecule). The amino acid residues or nucleotides at corresponding amino acid positions or nucleotide positions are then compared. When a position in one sequence is occupied by the same amino acid residue or nucleotide as the corresponding position in the other sequence, then the molecules are homologous at that position. As is known in the art, the percent identity between the two sequences is a function of the number of identical positions shared by the sequences, taking into account the number of gaps, and the length of each gap, which need to be introduced for optimal alignment of the two sequences. Sequence homology for polypeptides is typically measured using sequence analysis software.

In aspects, the present disclosure also encompasses polypeptides having a lower degree of identity but having sufficient similarity so as to perform one or more of the same functions performed by a polypeptide of the instant disclosure (e.g., a polypeptide having a sequence comprising, consisting of, or consisting essentially of one or more of SEQ ID NOS: 1-65 as disclosed herein, a concatemeric polypeptide as disclosed herein, including a concatemeric polypeptide comprising, consisting of, or consisting essentially of one or more of SEQ ID NOS: 66-67 and 70-71, or a polypeptide encoded by a nucleic acid molecule of the present disclosure, including a nucleic acid molecule having a sequence comprising, consisting of, or consisting essentially of one of more of SEQ ID NOS 68, 69, 72, 73-77). Similarity is determined by conserved amino acid substitution. Such substitutions are those that substitute a given amino acid in a polypeptide by another amino acid of like characteristics. Conservative substitutions are likely to be phenotypically silent. Typically seen as conservative substitutions are the replacements, one for another, among the aliphatic amino acids Ala, Val, Leu, Met, and Ile; interchange of the hydroxyl residues Ser and Thr, exchange of the acidic residues Asp and Glu, substitution between the amide residues Asn and Gln, exchange of the basic residues His, Lys and Arg and replacements among the aromatic residues Trp, Phe and Tyr. Guidance concerning which amino acid changes are likely to be phenotypically silent are found (Bowie JU et al., (1990), Science, 247(4948): 130610, which is herein incorporated by reference in its entirety).

In aspects, a variant polypeptide can differ in amino acid sequence by one or more substitutions, deletions, insertions, inversions, fusions, and truncations or a combination of any of these. Variant polypeptides can be fully functional (e.g., retain MHC binding propensity and/or TCR specificity, and/or retain ASFV activity) or can lack function in one or more activities. Fully functional variants typically contain only conservative variation or variation in non-critical residues or in non-critical regions; in this case, typically MHC contact residues providing MHC binding is preserved. Functional variants can also contain substitution of similar amino acids that result in no change or an insignificant change in function (e.g., retain MHC binding propensity and/or TCR specificity, and/or retain ASFV activity). In aspects, a variant and/or a homologous polypeptide retains the desired anti-ASFV activity of the instant disclsoure (e.g.: capable of stimulating, inducing, and/or expanding an immune response to ASFV (e.g., a cellular or humoral immune response to ASFV) and/or associated diseases in a subject; capable of stimulating, inducing, and/or expanding an ASFV-specific IFNγ response (e.g., by lymphocytes such as effector CD4+ and/or CD8+ T-cells), and/or capable of inducing immunity against ASFV as described herein). Alternatively, such substitutions can positively or negatively affect function to some degree. Non-functional variants typically contain one or more non-conservative amino acid substitutions, deletions, insertions, inversions, or truncation or a substitution, insertion, inversion, or deletion in a critical residue or critical region; in this case, typically TCR contact residues.

In aspects, funcational variants of a polypeptide having a sequence (or a core sequence) comprising, consisting of, or consisting essentially of one or more of SEQ ID NOS: 1-65 as disclosed herein may contain one or more conservative substitutions, and in aspects one or more non-conservative substitutions, at amino acid residues which are not believed to be essential for functioning (e.g., retain MHC binding propensity and/or TCR specificity, and/or retain ASFV activity) of the instantly-disclosed polypeptides. For example, in aspects, a variant polypeptide having a sequence (or a core sequence) comprising, consisting of, or consisting essentially of one or more of SEQ ID NOS: 1-65 as disclosed herein may contain one or more conservative substitutions (and in aspects, a nonconservative subsitution) in one or more SLA contact residues, provided SLA binding is preserved. MHC binding assays are well known in the art. In aspects, such assays may include the testing of binding affinity with respect to MHC class I and class II alleles in in vitro binding assays, with such binding assays as are known in the art. Exampels include, e.g., the soluble binding assays as disclosed in U.S. 7,884,184 or PCT/US2020/020089, both of which are herein incorporated by reference in their entireties. Additionally, in aspects, a fully functional variant polypeptide having a sequence (or a core sequence) comprising, consisting of, or consisting essentially of one or more of SEQ ID NOS: 1-65 as disclosed herein do not contain mutations at one or more critical residues or regions, such as TCR contact residues.

In aspects, the present disclosure also includes fragments of the instantly-disclosed polypeptides and concatemeric polypeptides. In aspects, the present disclosure also encompasses fragments of the variants of the instantly-disclosed polypeptides and concatemeric polypeptides. In aspects, as used herein, a fragment comprises at least about nine contiguous amino acids. Useful fragments (and fragments of the variants of the polypeptides and concatemeric polypeptides described herein) include those that retain one or more of the biological activities, particularly: MHC binding propensity and/or TCR specificity, and/or anti-ASFV activity. Biologically active fragments are, for example, about 9, 12, 15, 16, 20, 30, 40, 50, 60, 70, 80, 90, 100 or more amino acids in length, including any value or range therebetween. Fragments can be discrete (not fused to other amino acids or polypeptides) or can be within a larger polypeptide. Several fragments can be comprised within a single larger polypeptide. In aspects, a fragment designed for expression in a host can have heterologous pre-and pro-polypeptide regions fused to the amino terminus of the polypeptide fragment and an additional region fused to the carboxyl terminus of the fragment.

In aspects, the instantly disclosed polypeptides and concatemeric polypeptides of the present disclosure can include allelic or sequence variants (“mutants”) or analogs thereof, or can include chemical modifications (e.g., pegylation, glycosylation). In aspects, a mutant retains the same function, particularly MHC binding propensity and/or TCR specificity, and/or anti-ASFV activity. In aspects, a mutant can provide for enhanced binding to MHC molecules. In aspects, a mutant can lead to enhanced binding to TCRs. In another instance, a mutant can lead to a decrease in binding to MHC molecules and/or TCRs. Also contemplated is a mutant that binds, but does not allow signaling via the TCR.

The manner of producing the polypeptides of the present disclosure will vary widely, depending upon the nature of the various elements comprising the molecule. For example, an isolated polypeptide can be purified from cells that naturally express it, purified from cells that have been altered to express it (recombinant), or synthesized using known protein synthesis methods. The synthetic procedures may be selected so as to be simple, provide for high yields, and allow for a highly purified stable product. For example, polypeptides of the instant disclosure can be produced either from a nucleic acid disclosed herein, or by the use of standard molecular biology techniques, such as recombinant techniques, mutagenesis, or other known means in the art. An isolated polypeptide can be purified from cells that naturally express it, purified from cells that have been altered to express it (recombinant), or synthesized using known protein synthesis techniques. In aspects, a polypeptide of the instant disclosure is produced by recombinant DNA or RNA techniques. In aspects, a polypeptide of the instant disclosure can be produced by expression of a recombinant nucleic acid of the instant disclosure in an appropriate host cell. For example, a nucleic acid molecule encoding the polypeptide is cloned into an expression cassette or expression vector, the expression cassette or expression vector introduced into a host cell and the polypeptide expressed in the host cell . The polypeptide can then be isolated from the cells by an appropriate purification scheme using standard protein purification techniques. Alternatively a polypeptide can be produced by a combination of ex vivo procedures, such as protease digestion and purification. Further, polypeptides of the instant disclosure can be produced using site-directed mutagenesis techniques, or other mutagenesis techniques known in the art (see e.g., James A. Brannigan and Anthony J. Wilkinson., 2002, Protein engineering 20 years on. Nature Reviews Molecular Cell Biology 3, 964-970; Turanli-Yildiz B. et al., 2012, Protein Engineering Methods and Applications, intechopen.com, which are herein incorporated by reference in their entirety).

In aspects, the present disclosure also provides chimeric or fusion polypeptides. In aspects, the present disclosure provides a chimeric or fusion polypeptide (which in aspects may be isolated, synthetic, and/or recombinant) wherein one or more of the instantly-disclosed polypeptides or concatemeric polypeptides is a part thereof. In aspects, a chimeric or fusion polypeptide composition comprises one or more polypeptides of the present disclosure joined to, linked to (e.g., fused in-frame, chemically-linked, or otherwise bound), and/or inserted into a heterologous polypeptide. As previously described, with respect to the one or more T-cell epitopes of the instant disclosure, the term “heterologous polypeptide” is intended to mean that the one or more T-cell epitopes of the instant disclosure are heterologous to, or not included naturally, in the heterologous polypeptide. In aspects, one or more of the instantly-disclosed polypeptides may be inserted into the heterologous polypeptide (e.g., through recombinant techniques, mutagenesis, or other known means in the art), may be added to the C-terminus (with or without the use of linkers, as is known in the art), and/or added to the N-terminus (with or without the use of linkers, as is known in the art) of the heterologous polypeptide. For example, protein engineering by mutagenesis can be performed using site-directed mutagenesis techniques, or other mutagenesis techniques known in the art (see e.g., James A. Brannigan and Anthony J. Wilkinson., 2002, Protein engineering 20 years on. Nature Reviews Molecular Cell Biology 3, 964-970; Turanli-Yildiz B. et al., 2012, Protein Engineering Methods and Applications, intechopen.com, which are herein incorporated by reference in their entirety). In aspects, chimeric or fusion polypeptides comprise one or more of the instantly-disclosed polypeptides operatively linked to a heterologous polypeptide. “Operatively linked” indicates that the one or more of the instantly-disclosed polypeptides and the heterologous polypeptide are fused in-frame or chemically-linked or otherwise bound. For example, in aspects of the above isolated, synthetic, and/or recombinant chimeric or fusion polypeptide compositions, the one or more polypeptides of the present disclosure have a sequence comprising, consisting of, or consisting essentially of one or more of SEQ ID NOS: 1-67, 70-71 (and/or fragments or variants thereof). In aspects of the chimeric or fusion polypeptide compositions, the one or more peptides or polypeptides of the instant disclosure may be joined to, linked to (e.g., fused in-frame, chemically-linked, or otherwise bound), and/or inserted into a heterologous polypeptide as a whole, although it may be made up from a joined to, linked to (e.g., fused in-frame, chemically-linked, or otherwise bound), and/or inserted amino acid sequence, together with flanking amino acids of the heterologous polypeptide. In aspects, a chimeric or fusion polypeptide composition comprises a polypeptide of the instant disclosure having a sequence comprising one or more of SEQ ID NOS: 1-65 (and/or fragments or variants thereof, and optionally 1 to 12 additional amino acids distributed in any ratio on the N terminus and/or C-terminus of the polypeptide of SEQ ID NOS: 1-65), wherein said one or more of SEQ ID NOS: 1-65 is not naturally included in the polypeptide and/or said of one or more of SEQ ID NOS: 1-65 is not located at its natural position in the polypeptide. In aspects, the one or more the one or more of peptide or polypeptides of the present disclosure can be joined, linked to (e.g., fused in-frame, chemically-linked, or otherwise bound), and/or inserted into the heterologous polypeptide. In aspects, chimeric or fusion polypeptide compositions comprise one or more of the instantly-disclosed T-cell epitopes operatively linked to a heterologous polypeptide having an amino acid sequence not substantially homologous to the T-cell epitope. In aspects, the chimeric or fusion polypeptide does not affect function of the T-cell epitope per se. For example, the fusion polypeptide can be a GST-fusion polypeptide in which the T-cell epitope sequences are fused to the C-terminus of the GST sequences. Other types of fusion polypeptides include, but are not limited to, enzymatic fusion polypeptides, for example beta-galactosidase fusions, yeast two-hybrid GAL fusions, poly-His fusions and Ig fusions. Such fusion polypeptides, particularly poly-His fusions or affinity tag fusions, can facilitate the purification of recombinant polypeptide. In certain host cells (e.g., mammalian host cells), expression and/or secretion of a polypeptide can be increased by using a heterologous signal sequence. Therefore, in aspects, the chimeric or fusion polypeptide contains a heterologous signal sequence at its N-terminus. In aspects of the above chimeric or fusion polypeptide compositions, the heterologous polypeptide or polypeptide comprises a biologically active molecule. In aspects, the biologically active molecule is selected from the group consisting of an immunogenic molecule, a T-cell epitope, a viral protein, and a bacterial protein. In aspects, the one or more peptides or polypeptides of the instant disclosure (e.g., but not limited to, a peptide or polypeptide comprising, consisting, or consisting essentially of an amino acid sequence of SEQ ID NOS:NOS: 1-65 (and/or fragments or variants thereof), and optionally 1 to 12 additional amino acids distributed in any ratio on the N terminus and/or C-terminus of the polypeptide of SEQ ID NOS:NOS: 1-65) can be joined or linked to (e.g., fused in-frame, chemically-linked, or otherwise bound) to a small molecule, drug, or drug fragment. For example, one or more peptides or polypeptides of the instant disclosure (e.g., but not limited to, a peptide or polypeptide comprising, consisting, or consisting essentially of an amino acid sequence of SEQ ID NOS:NOS: 1-65 (and/or fragments or variants thereof), and optionally 1 to 12 additional amino acids distributed in any ratio on the N terminus and/or C-terminus of the polypeptide of SEQ ID NOS:NOS: 1-65) can be joined or linked to (e.g., fused in-frame, chemically-linked, or otherwise bound) a drug or drug fragment that is binds with high affinity to defined SLAs. In aspects of the above-described chimeric or fusion polypeptide compositions, the chimeric or fusion polypeptide compositions can be recombinant, isolated, and/or synthetic.

A chimeric or fusion polypeptide composition can be produced by standard recombinant DNA or RNA techniques as are known in the art. For example, DNA or RNA fragments coding for the different polypeptide sequences may be ligated together in-frame in accordance with conventional techniques. In another embodiment, the fusion gene can be synthesized by conventional techniques including automated DNA synthesizers. Alternatively, polymerase chain reaction (PCR) amplification of nucleic acid fragments can be carried out using anchor primers which give rise to complementary overhangs between two consecutive nucleic acid fragments which can subsequently be annealed and re-amplified to generate a chimeric nucleic acid sequence(Short Protocols in Molecular Biology: A Compendium of Methods from Current Protocols in Molecular Biology, (2^{ND}, 1992), FM Asubel et al. (eds), Green Publication Associates, New York, NY (Publ), ISBN: 9780471566355, which is herein incorporated by reference in its entirety). Further, one or more polypeptides of the present disclosure (e.g., one or more T-cell epitopes of the present disclosure having a sequence comprising, consisting of, or consisting essentially of one or more of SEQ ID NOS: 1-65 (and/or fragments or variants thereof), and optionally 1 to 12 additional amino acids distributed in any ratio on the N terminus and/or C-terminus of the polypeptide of SEQ ID NOS: 1-65) can be inserted into a heterologous polypeptide or inserted into a non-naturally occurring position of a polypeptide through recombinant techniques, synthetic polymerization techniques, mutagenesis, or other standard techniques known in the art. For example, protein engineering by mutagenesis can be performed using site-directed mutagenesis techniques, or other mutagenesis techniques known in the art (see e.g., James A. Brannigan and Anthony J. Wilkinson., 2002, Protein engineering 20 years on. Nature Reviews Molecular Cell Biology 3, 964-970; Turanli-Yildiz B. et al., 2012, Protein Engineering Methods and Applications, intechopen.com, which are herein incorporated by reference in their entirety).

Moreover, many expression vectors are commercially available that already encode a fusion moiety (e.g., a GST protein). A nucleic acid molecule encoding a T-cell epitope of the invention can be cloned into such an expression vector such that the fusion moiety is linked in-frame to the at least one T-cell epitope.

In aspects, the polypeptides, concatemeric polypeptides, and chimeric or fusion polypeptides can be purified to homogeneity or partially purified. It is understood, however, that preparations in which the T-cell epitope compounds and compositions are not purified to homogeneity are useful. The critical feature is that the preparation allows for the desired function of the composition, even in the presence of considerable amounts of other components. Thus, the present disclosure encompasses various degrees of purity. In one embodiment, the language “substantially free of cellular material” includes preparations of the polypeptides, concatemeric polypeptides, and chimeric or fusion polypeptides having less than about 30% (by dry weight) other proteins (e.g., contaminating protein), less than about 20% other proteins, less than about 10% other proteins, less than about 5% other proteins, less than about 4% other proteins, less than about 3% other proteins, less than about 2% other proteins, less than about 1% other proteins, or any value or range therebetween.

In aspects, when a polypeptide, concatemeric polypeptide, and chimeric or fusion polypeptide of the present disclosure is recombinantly produced, the composition can also be substantially free of culture medium, for example, culture medium represents less than about 20%, less than about 10%, or less than about 5% of the volume of the the polypeptides, concatemeric polypeptides, and chimeric or fusion polypeptides preparation. The language “substantially free of chemical precursors or other chemicals” includes preparations of the the polypeptides, concatemeric polypeptides, and chimeric or fusion polypeptides in which it is separated from chemical precursors or other chemicals that are involved in the T-cell epitope’s synthesis. The language “substantially free of chemical precursors or other chemicals” can include, for example, preparations of the the polypeptides, concatemeric polypeptides, and chimeric or fusion polypeptides having less than about 30% (by dry weight) chemical precursors or other chemicals, less than about 20% chemical precursors or other chemicals, less than about 10% chemical precursors or other chemicals, less than about 5% chemical precursors or other chemicals, less than about 4% chemical precursors or other chemicals, less than about 3% chemical precursors or other chemicals, less than about 2% chemical precursors or other chemicals, or less than about 1% chemical precursors or other chemicals.

In aspects, the present disclosure also includes pharmaceutically acceptable salts of the T-cell epitope compounds and compositions (including one or more of e.g., peptides or polypeptides as disclosed herein; concatemeric peptides as disclosed herein; chimeric or fusion polypeptide compositions as disclosed herein (which in aspects may be isolated, synthetic, and/or recombinant). “Pharmaceutically acceptable salt” means a salt that is pharmaceutically acceptable and that possesses the desired pharmacological activity of the parent peptide or polypeptide (e.g., peptides, polypeptides, concatemeric peptides, and/or chimeric or fusion polypeptides as disclosed herein). As used herein, “pharmaceutically acceptable salt” refers to derivative of the instantly-disclosed polypeptides, concatemeric polypeptides, and/or chimeric or fusion polypeptides, wherein such compounds are modified by making acid or base salts thereof. Examples of pharmaceutically acceptable salts include, but are not limited to, mineral or organic acid salts of basic residues such as amines, alkali or organic salts of acidic residues such as carboxylic acids, and the like. The pharmaceutically acceptable salts include the conventional non-toxic salts or the quaternary ammonium salts of the parent compound formed, for example, from non-toxic inorganic or organic acids. For example, such conventional non-toxic salts include, but are not limited to, those derived from inorganic and organic acids selected from 2-acetoxybenzoic, 2-hydroxyethane sulfonic, acetic, ascorbic, benzene sulfonic, benzoic, bicarbonic, carbonic, citric, edetic, ethane disulfonic, 1,2-ethane sulfonic, fumaric, glucoheptonic, gluconic, glutamic, glycolic, glycollyarsanilic, hexylresorcinic, hydrabamic, hydrobromic, hydrochloric, hydroiodic, hydroxymaleic, hydroxynaphthoic, isethionic, lactic, lactobionic, lauryl sulfonic, maleic, malic, mandelic, methane sulfonic, napsylic, nitric, oxalic, pamoic, pantothenic, phenylacetic, phosphoric, polygalacturonic, propionic, salicyclic, stearic, subacetic, succinic, sulfamic, sulfanilic, sulfuric, tannic, tartaric, toluene sulfonic, and the commonly occurring amine acids, e.g., glycine, alanine, phenylalanine, arginine, etc.

### Nucleic Acids

In aspects, the present disclosure also provides for nucleic acids (e.g., DNAs (including cDNA), RNAs (such as, but limited to mRNA), plasmids, vectors, viruses, or hybrids thereof, all of which may be isolated, synthetic, and/or recombinant) that encode in whole or in part one or more peptides, polypeptides, concatemeric peptides, and/or chimeric or fusion polypeptides of the present disclosure. The nucleic acid may be used to produce the one or more peptides, polypeptides, concatemeric peptides, and/or chimeric or fusion polypeptides as described herein in vitro, or to produce cells expressing the polypeptide on their surface, or to produce vaccines wherein the active agent is the nucleic acid or a plasmid or vector containing the nucleic acid.

For example, in aspects, the instant disclosure is directed to a nucleic acid encoding a peptide or polypeptide comprising, consisting of, or consisting essentially of one or more peptides or polypeptides having an amino acid sequence of SEQ ID NOS: 1-65 (and/or fragments or variants thereof), and optionally 1 to 12 additional amino acids distributed in any ratio on the N terminus and/or C-terminus of the polypeptide of SEQ ID NOS: 1-65, including fragments and variants thereof as described herein. Additionally, in aspects, the instant disclosure is directed to a nucleic acid encoding a concatemeric polypeptide comprising, consisting of, or consisting essentially of one or more of SEQ ID NOS: 66-67 and 70-71, including variants and fragments thereof as described herein.

In aspects, the present disclosure provides a nucleic acid having a sequence comprising, consisting of, or consisting essentially of one of more of SEQ ID NOS 68, 69, 72, 73 (and fragments or variants thereof). In aspects, the present disclosure provides a nucleic acid with at least 60%, 70%, 80%, 90%, or 95% homology to SEQ ID NO 68. In aspects, the present disclosure provides a nucleic acid with at least 60%, 70%, 80%, 90%, or 95% homology to SEQ ID NO 68, provided said polypeptide encoded by said nucleic retains anti-ASFV activity. In aspects, the present disclosure provides a nucleic acid with at least 60%, 70%, 80%, 90%, or 95% homology to SEQ ID NO 69. In aspects, the present disclosure provides a nucleic acid with at least 60%, 70%, 80%, 90%, or 95% homology to SEQ ID NO 69, provided said polypeptide encoded by said nucleic acid retains anti-ASFV activity. In aspects, the present disclosure provides a nucleic acid with at least 60%, 70%, 80%, 90%, or 95% homology to SEQ ID NO 72. In aspects, the present disclosure provides a nucleic acid with at least 60%, 70%, 80%, 90%, or 95% homology to SEQ ID NO 72, provided said polypeptide encoded by said nucleic acid retains anti-ASFV activity. In aspects, the present disclosure provides a nucleic acid with at least 60%, 70%, 80%, 90%, or 95% homology to SEQ ID NO 73. In aspects, the present disclosure provides a nucleic acid with at least 60%, 70%, 80%, 90%, or 95% homology to SEQ ID NO 73, provided said polypeptide encoded by said nucleic acid retains anti-ASFV activity. In aspects, said nucleic acids are DNA. In aspects, said nucleic acids may be included in a plasmid or vector.

In aspects, the present disclosure provides a nucleic acid having a sequence comprising, consisting of, or consisting essentially of one of more of SEQ ID NOS 74-77 (and fragments or variants thereof). In aspects, the present disclosure provides a nucleic acid with at least 60%, 70%, 80%, 90%, or 95% homology to SEQ ID NO 74. In aspects, the present disclosure provides a nucleic acid with at least 60%, 70%, 80%, 90%, or 95% homology to SEQ ID NO 74, provided said polypeptide encoded by said nucleic acid retains anti-ASFV activity. In aspects, the present disclosure provides a nucleic acid with at least 60%, 70%, 80%, 90%, or 95% homology to SEQ ID NO 75. In aspects, the present disclosure provides a nucleic acid with at least 60%, 70%, 80%, 90%, or 95% homology to SEQ ID NO 75, provided said polypeptide encoded by said nucleic retains anti-ASFV activity. In aspects, the present disclosure provides a nucleic acid with at least 60%, 70%, 80%, 90%, or 95% homology to SEQ ID NO 76. In aspects, the present disclosure provides a nucleic acid with at least 60%, 70%, 80%, 90%, or 95% homology to SEQ ID NO 76, provided said polypeptide encoded by said nucleic retains anti-ASFV activity. In aspects, the present disclosure provides a nucleic acid with at least 60%, 70%, 80%, 90%, or 95% homology to SEQ ID NO 77. In aspects, the present disclosure provides a nucleic acid with at least 60%, 70%, 80%, 90%, or 95% homology to SEQ ID NO 77, provided said polypeptide encoded by said nucleic retains anti-ASFV activity. In aspects, said nucleic acids are RNA (e.g., mRNA). In aspects, said nucleic acids may be included in a plasmid or vector.

For polynucleotides, a “variant” comprises a deletion and/or addition of one or more nucleotides at one or more internal sites within the polynucleotide sequences of the instant disclosure and/or a substitution of one or more nucleotides at one or more sites in the polynucleotide sequences of the instant disclosure. One of skill in the art will recognize that variants of the polynucleotides of the invention will be constructed such that the open reading frame is maintained. For polynucleotides, conservative variants include those sequences that, because of the degeneracy of the genetic code, encode the amino acid sequence of one of the polypeptides of the instant disclousre. Naturally occurring allelic variants such as these can be identified with the use of well-known molecular biology techniques, as, for example, with polymerase chain reaction (PCR) and hybridization techniques as outlined below. Variant polynucleotides also include synthetically derived polynucleotides, such as those generated, for example, by using site-directed mutagenesis but which still encode a polynucleotide having the desired activity of the instant disclosure (i.e., encoding a polypeptide that possesses the desired biological activity, that is, MHC binding propensity and/or TCR specificity, or anti-ASFV activity, as described herein). Generally, variants of a particular polynucleotide of the invention will have at least about 40%, 45%, 50%, 55%, 60%, 65%, 70%, 75%, 80%, 85%, 90%, 91%, 92%, 93%, 94%, 95%, 96%, 97%, 98%, 99% or more sequence identity to that particular polynucleotide as determined by sequence alignment programs and parameters described elsewhere herein.

Variants of a particular polynucleotide of the instant disclosure (i.e., the reference polynucleotide) can also be evaluated by comparison of the percent sequence identity between the polypeptide encoded by a variant polynucleotide and the polypeptide encoded by the reference polynucleotide. Thus, for example, an isolated polynucleotide that encodes a polypeptide with a given percent sequence identity to a polypeptide of SEQ ID NOs: 1-67, 70-71 (including the noted mutations/modifications) are disclosed. Percent sequence identity between any two polypeptides can be calculated using sequence alignment programs and parameters described elsewhere herein. Where any given pair of polynucleotides of the invention is evaluated by comparison of the percent sequence identity shared by the two polypeptides they encode, the percent sequence identity between the two encoded polypeptides is at least about 40%, 45%, 50%, 55%, 60%, 65%, 70%, 75%, 80%, 85%, 90%, 91%, 92%, 93%, 94%, 95%, 96%, 97%, 98%, 99% or more sequence identity.

In aspects, the nucleic acid further comprises, or is contained within, an expression cassette, a plasmid, and expression vector, or recombinant virus, wherein optionally the nucleic acid, or the expression cassette, plasmid, expression vector, or recombinant virus is contained within a cell, optionally a human cell or a non-human cell, and optionally the cell is transformed with the nucleic acid, or the expression cassette, plasmid, expression vector, or recombinant virus. In aspects, the cell can be a mammalian cell, bacterial cell, insect cell, or yeast cell. In aspects, the nucleic acid molecules of the present disclosure can be inserted into vectors and used, for example, as expression vectors or gene therapy vectors. Gene therapy vectors can be delivered to a subject by, e.g., intravenous injection, local administration (U.S. Pat. No. 5,328,470) or by stereotactic injection (Chen SH et al., (1994), Proc Natl Acad Sci USA, 91(8):3054-7, which are herein incorporated by reference in their entirety). Similarly, the nucleic acid molecules of the present disclosure can be inserted into plasmids. For example, in aspects, the present disclosure is directed to a plasmid having a sequence comprising, consisting of, or consisting essentially of one of more of SEQ ID NOS: 69, 73, 75, and 77, as described above. The pharmaceutical preparation of the gene therapy vector or plasmid can include the gene therapy vector or plasmid in an acceptable excipient and/or carrier (and may also include an optional adjuvant), or can comprise a slow release matrix in which the gene delivery vehicle is imbedded. Alternatively, where the complete gene delivery vector can be produced intact from recombinant cell s, e.g., retroviral vectors, the pharmaceutical preparation can include one or more cells that produce the gene delivery system. Such pharmaceutical compositions can be included in a container, pack, or dispenser together with instructions for administration.

The nucleic acid of the instant disclosure may be DNAs (including but not limited to cDNA) or RNAs (including but not limited to mRNA), single- or double-stranded. The nucleic acid is typically DNA or RNA (including mRNA). The nucleic acid may be produced by techniques well known in the art, such as synthesis, or cloning, or amplification of the sequence encoding the immunogenic polypeptide; synthesis, or cloning, or amplification of the sequence encoding the cell membrane addressing sequence; ligation of the sequences and their cloning/amplification in appropriate vectors and cells.The nucleic acids provided herein (whether RNAs, DNAs, vectors, viruses or hybrids thereof) that encode in whole or in part one or more peptides, polypeptides, concatemeric peptides, and/or chimeric or fusion polypeptides as described herein can be isolated from a variety of sources, genetically engineered, amplified, synthetically produced, and/or expressed/generated recombinantly. Recombinant polypeptides generated from these nucleic acids can be individually isolated or cloned and tested for a desired activity. Any recombinant expression system can be used, including e.g. in vitro, bacterial, fungal, mammalian, yeast, insect or plant cell expression systems. In aspects nucleic acids provided herein are synthesized in vitro by well-known chemical synthesis techniques (as described in, e.g., Adams (1983) J. Am. Chem. Soc. 105:661; Belousov (1997) Nucleic Acids Res. 25:3440-3444; Frenkel (1995) Free Radic. Biol. Med. 19:373-380; Blommers (1994) Biochemistry 33:7886-7896; Narang (1979) Meth. Enzymol. 68:90; Brown (1979) Meth. Enzymol. 68:109; Beaucage (1981) Tetra. Lett. 22:1859; U.S. Pat. No. 4,458,066, all of which are herein incorporated by reference in their entirety). Further, techniques for the manipulation of nucleic acids provided herein, such as, e.g., subcloning, labeling probes (e.g., random-primer labeling using Klenow polymerase, nick translation, amplification), sequencing, hybridization and the like are well described in the scientific and patent literature (see, e.g., Sambrook, ed., MOLECULAR CLONING: A LABORATORY MANUAL (2ND ED.), Vols. 1-3, Cold Spring Harbor Laboratory, (1989); CURRENT PROTOCOLS IN MOLECULAR BIOLOGY, Ausubel, ed. John Wiley & Sons, Inc., New York (1997); LABORATORY TECHNIQUES IN BIOCHEMISTRY AND MOLECULAR BIOLOGY: HYBRIDIZATION WITH NUCLEIC ACID PROBES, Part I. Theory and Nucleic Acid Preparation, Tijssen, ed. Elsevier, N.Y. (1993), all of which are herein incorporated by reference in their entirety).

As previously mentioned, the nucleic acid molecules according to the present disclosure may be provided in the form of a nucleic acid molecule per se such as naked nucleic acid molecules; a plasmid, a vector; virus or host cell etc., either from prokaryotic or eukaryotic origin. Vectors include expression vectors that contain a nucleic acid molecule of the invention. The vectors of the present invention may, for example, comprise a transcriptional promoter, and/or a transcriptional terminator, wherein the promoter is operably linked with the nucleic acid molecule, and wherein the nucleic acid molecule is operably linked with the transcription terminator.

In aspects, the vector may be a viral vector comprising a nucleic acid as defined above. The viral vector may be derived from different types of viruses, such as, Swinepox, Fowlpox, Pseudorabies, Aujezky’s virus, salmonella, vaccinia virus, BHV (Bovine Herpes Virus), HVT (Herpes Virus of Turkey), adenovirus, TGEV (Transmissible Gastroenteritidis Coronavirus), Erythrovirus, and SIV (Simian Immunodeficiency Virus). Other expression systems and vectors may be used as well, such as plasmids that replicate and/or integrate in yeast cell s.

### Pharmaceutical Compositions and Formulations

In aspects, the T-cell epitope compounds and compositions of the present disclosure (including one or more of e.g., polypeptides as disclosed herein; concatemeric peptides as disclosed herein; chimeric of fusion polypeptide compositions as disclosed herein; nucleic acids as disclosed herein, including nucleic acids encoding such peptides, polypeptides, concatemeric peptides, or chimeric of fusion polypeptide compositions as disclosed herein; expression cassettes, plasmids, expression vectors, recombinant viruses, or cells as disclosed herein, and vaccines as disclosed herein; hereafter referred to as “T-cell epitope compounds and compositions of the present disclosure”) of the like may be comprised in a pharmaceutical composition or formulation. In aspects, the instantly disclosed pharmaceutical compositions or formulations generally comprise a T-cell epitope compound and composition of the present disclosure and a pharmaceutically-acceptable carrier and/or excipient. In aspects, said pharmaceutical compositions are suitable for administration. Pharmaceutically-acceptable carriers and/or excipients are determined in part by the particular composition being administered, as well as by the particular method used to administer the composition. Accordingly, there is a wide variety of suitable formulations of pharmaceutical compositions for administering the instantly-disclosed T-cell epitope compounds and compositions (see, e.g., Remington’s Pharmaceutical Sciences, (18^{TH} Ed, 1990), Mack Publishing Co., Easton, PA Publ). In aspects, the pharmaceutical compositions are generally formulated as sterile, substantially isotonic, and in full compliance with all Good Manufacturing Practice (GMP) regulations of the U.S. Food and Drug Administration. Pharmaceutical compositions as disclosed herein are able for use in stimulating, inducing, and/or expanding an immune response to ASFV and/or associated diseases, and can be used in methods of treating and/or preventing ASFV and/or associated diseases in a subject, such as a non-human animal (e.g., pigs)

The terms “pharmaceutically-acceptable,” “physiologically-tolerable,” and grammatical variations thereof, as they refer to compositions, carriers, excipients, and reagents, are used interchangeably and represent that the materials are capable of administration to or upon a subject without the production of undesirable physiological effects to a degree that would prohibit administration of the composition. For example, “pharmaceutically-acceptable excipient” means, for example, an excipient that is useful in preparing a pharmaceutical composition that is generally safe, non-toxic, and desirable, and includes excipients that are acceptable for veterinary use as well as for human pharmaceutical use. Such excipients can be solid, liquid, semisolid, or, in the case of an aerosol composition, gaseous . A person of ordinary skill in the art would be able to determine the appropriate timing, sequence and dosages of administration for particular T-cell epitope compounds and compositions of the present disclosure.

In aspects, preferred examples of such carriers or diluents include, but are not limited to, water, saline, Ringer’s solutions, dextrose solution, and 5% human serum albumin. Liposomes and non-aqueous vehicles such as fixed oils can also be used. The use of such media and compounds for pharmaceutically active substances is well known in the art. Except insofar as any conventional media or compound is incompatible with the T-cell epitope compounds and compositions of the present disclosure and as previously described above use thereof in the compositions is contemplated. Supplementary active compounds can also be incorporated into the compositions.

In aspects, T-cell epitope compounds and compositions of the present disclosure are formulated to be compatible with its intended route of administration. The T-cell epitope compounds and compositions of the present disclosure can be administered by parenteral, topical, intravenous, oral, subcutaneous, intraarterial, intradermal, transdermal, rectal, intracranial, intrathecal, intraperitoneal, intranasal; vaginally; intramuscular route or as inhalants. In aspects, T-cell epitope compounds and compositions of the present disclosure can be injected directly into a particular tissue where deposits have accumulated, e.g., intracranial injection. In other aspects, intramuscular injection or intravenous infusion may be used for administration of T-cell epitope compounds and compositions of the present disclosure. In some methods, T-cell epitope compounds and compositions of the present disclosure are administered as a sustained release composition or device, such as but not limited to a Medipad™ device. In aspects, T-cell epitope compounds and compositions of the present disclosure are administered intradermally, e.g., by using a commercial needle-free high-pressure device such as Pulse NeedleFree technology (Pulse 50TM Micro Dose Injection System, Pulse NeedleFree Systems; Lenexa, KS, USA). In aspects, said commercial needle-free high-pressure device (e.g., Pulse NeedleFree technology) confers one or more of the following benefits: non-invasive, reduces tissue trauma, reduces pain, requires a smaller opening in the dermal layer to deposit the composition in the subject (e.g., only requires a micro skin opening), instant dispersion of the composition, better absorption of the composition, greater dermal exposure to the composition, and/or reduced risk of sharps injury.

In aspects, T-cell epitope compounds and compositions of the present disclosure can optionally be administered in combination with other agents that are at least partly effective in treating various medical conditions as described herein. For example, in the case of administration into the central nervous system of a subject, T-cell epitope compounds and compositions of the present disclosure can also be administered in conjunction with other agents that increase passage of the agents of the invention across the blood-brain barrier.

In aspects, solutions or suspensions used for parenteral, intradermal, or subcutaneous application can include, but are not limited to, the following components: a sterile diluent such as water for injection, saline solution, fixed oils, polyethylene glycols, glycerine, propylene glycol or other synthetic solvents; antibacterial compounds such as benzyl alcohol or methyl parabens; antioxidants such as ascorbic acid or sodium bisulfite; chelating compounds such as ethylenediaminetetraacetic acid (EDTA); buffers such as acetates, citrates or phosphates, and compounds for the adjustment of tonicity such as sodium chloride or dextrose. The pH can be adjusted with acids or bases, such as hydrochloric acid or sodium hydroxide. Examples of excipients can include starch, glucose, lactose, sucrose, gelatin, malt, rice, flour, chalk, silica gel, water, ethanol, DMSO, glycol, propylene, dried skim milk, and the like. The composition can also contain pH buffering reagents, and wetting or emulsifying agents.

In aspects, pharmaceutical compositions or formulations suitable for injectable (including by an intradermal needle-free high-pressure device) use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor ELTM (BASF, Parsippany, NJ) or phosphate buffered saline (PBS). In all cases, the composition is sterile and should be fluid to the extent that easy syringeability exists. It is stable under the conditions of manufacture and storage and is preserved against the contaminating action of microorganisms such as bacteria and fungi. In aspects formulations including a T-cell epitope compound or composition of the present disclosure may include aggregates, fragments, breakdown products and post-translational modifications, to the extent these impurities bind SLA and present the same TCR face to cognate T-cells they are expected to function in a similar fashion to pure T-cell epitopes. The carrier can be a solvent or dispersion medium containing, e.g., water, ethanol, polyol (e.g., glycerol, propylene glycol, and liquid polyethylene glycol, and the like), and suitable mixtures thereof. The proper fluidity can be maintained, e.g., by the use of a coating such as lecithin, by the maintenance of the required particle size in the case of dispersion and by the use of surfactants. Prevention of the action of microorganisms can be achieved by various antibacterial and antifungal compounds, e.g., parabens, chlorobutanol, phenol, ascorbic acid, thimerosal, and the like. In many cases, it will be preferable to include isotonic compounds, e.g., sugars, polyalcohols such as mannitol, sorbitol, sodium chloride, and the like in the composition. Prolonged absorption of the injectable compositions can be brought about by including in the composition a compound that delays absorption, e.g., aluminum monostearate and gelatin.

In aspects, sterile solutions suitable for injectable and/or intradermal needle-free high-pressure device use can be prepared by incorporating the T-cell epitope compounds and compositions of the present disclosure in the required amount in an appropriate solvent with one or a combination of ingredients enumerated above, as required, followed by filtered sterilization. Generally, dispersions are prepared by incorporating the binding agent into a sterile vehicle that contains a basic dispersion medium and the required other ingredients from those enumerated above. In the case of sterile powders for the preparation of sterile solutions suitable for injectable and/or intradermal needle-free high-pressure device use, methods of preparation are vacuum drying and freeze-drying that yields a powder of the active ingredient plus any additional desired ingredient from a previously sterile-filtered solution thereof. Further, T-cell epitope compounds and compositions of the present disclosure can be administered in the form of a depot injection or implant preparation that can be formulated in such a manner as to permit a sustained or pulsatile release of the active ingredient.

In aspects, oral compositions generally include an inert diluent or an edible carrier and can be enclosed in gelatin capsules or compressed into tablets. In aspects, for the purpose of oral therapeutic administration, the binding agent can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding compounds, and/or adjuvant materials can be included as part of the composition. In aspects, the tablets, pills, capsules, troches and the like can contain any of the following ingredients, or compounds of a similar nature: a binder such as microcrystalline cellulose, gum tragacanth or gelatin; an excipient such as starch or lactose, a disintegrating compound such as alginic acid, Primogel or corn starch; a lubricant such as magnesium stearate or Sterotes; a glidant such as colloidal silicon dioxide; a sweetening compound such as sucrose or saccharin; or a flavoring compound such as peppermint, methyl salicylate or orange flavoring.

For administration by inhalation, T-cell epitope compunds and compositions of the present disclosure can be delivered in the form of an aerosol spray from pressured container or dispenser that contains a suitable propellant, e.g., a gas such as carbon dioxide, or a nebulizer.

In aspects, systemic administration of the T-cell epitope compounds and compositions of the present disclosure can also be by transmucosal or transdermal means. For transmucosal or transdermal administration, penetrants appropriate to the barrier to be permeated are used in the formulation. Such penetrants are generally known in the art, and include, e.g., for transmucosal administration, detergents, bile salts, and fusidic acid derivatives. Transmucosal administration can be accomplished through the use of nasal sprays or suppositories. For transdermal administration, the T-cell epitope compounds and compositions of the present disclosure may be formulated into ointments, salves, gels, or creams and applied either topically or through transdermal patch technology as generally known in the art.

In aspects, the T-cell epitope compounds and compositions of the present disclosure can also be prepared in the form of suppositories (e.g., with conventional suppository bases such as cocoa butter and other glycerides) or retention enemas for rectal delivery.

In aspects, the T-cell epitope compounds and compositions of the present disclosure are prepared with carriers that protect the T-cell epitope compounds and compositions against rapid elimination from the body, such as a controlled-release formulation, including implants and microencapsulated delivery systems. Biodegradable, biocompatible polymers can be used, such as, for example, ethylene vinyl acetate, polyanhydrides, polyglycolic acid, collagen, polyorthoesters, and polylactic acid. Methods for preparation of such formulations will be apparent to those skilled in the art. The materials can also be obtained commercially, e.g., from Alza Corporation and Nova Pharmaceuticals, Inc. Liposomal suspensions (including liposomes targeted to infected cells with monoclonal antibodies to viral antigens) can also be used as pharmaceutically-acceptable carriers. These can be prepared according to methods known to those skilled in the art (U.S. Pat. No. 4,522,811, which is herein incorporated by reference in its entirety). In aspects, the T-cell epitope compounds and compositions of the present disclosure can be implanted within or linked to a biopolymer solid support that allows for the slow release of the T-cell epitope compounds and compositions to the desired site.

In aspects, it is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of binding agent calculated to produce the desired therapeutic effect in association with the required pharmaceutical carrier. The specification for the dosage unit forms of the instant disclosure are dictated by and directly dependent on the unique characteristics of the binding agent and the particular therapeutic effect to be achieved, and the limitations inherent in the art of compounding such T-cell epitope compounds and compositions for the treatment of a subject.

### Vaccine Compositions

The term “vaccine” as used herein includes an agent which may be used to cause, stimulate or amplify the immune system of animals (e.g., pigs) against a pathogen. Vaccines of the invention are able to cause or stimulate or amplify immunity against an ASFV infection and related diseases caused by ASFV, including ASF.

The term “immunization” includes the process of delivering an immunogen to a subject. Immunization may, for example, enable a continuing high level of antibody and/or cellular response in which T-lymphocytes can kill or suppress the pathogen in the immunized non-human animal, such as pig, which is directed against a pathogen or antigen to which the animal has been previously exposed.

Vaccines of the instant disclosure comprise an immunologically effective amount of a T-cell epitope compound or composition of the instant disclosure, and in aspects in a pharmaceutically acceptable vehicle and optionally with additional excipients and/or an adjuvant. As a result of the vaccination with a composition of the present disclosure, animals become at least partially or completely immune to ASFV infections, or resistant to developing moderate or severe ASFV infections and/or ASFV related diseases. The instantly-disclosed ASFV vaccines may be used to elicit a humoral and/or a cellular response, including CD4+ and CD8+ T effector cell responses. ASFV infections or associated diseases include, for example, African Swine Fever. Preferably, a non-human animal subject, such as a pig, is protected to an extent to which one to all of the adverse physiological symptoms or effects of ASFV infections are significantly reduced, ameliorated or prevented.

In practice, the exact amount required for an immunologically effective dose may vary from subject to subject depending on factors such as the age and general condition of the subject, the nature of the formulation and the mode of administration. An appropriate “effective amount” may be determined by one of ordinary skill in the art using only routine experimentation. For instance, methods are known in the art for determining or titrating suitable dosages of a vaccine to find minimal effective dosages based on the weight of the non-human animal subject, concentration of the vaccine and other typical factors. The dosage of the vaccines of the present disclosure will depend on the species, breed, age, size, vaccination history, and health status of the animal (e.g., swine/pig) to be vaccinated, as well as the route of administration, e.g., subcutaneous, intradermal, oral intramuscular or intravenous administration. The vaccines of the instant disclosure can be administered as single doses or in repeated doses. The vaccines of the instant disclosure can be administered alone, or can be administered simultaneously or sequentially administered with one or more further compositions, such as other porcine immunogenic or vaccine compositions. Where the compositions are administered at different times, the administrations may be separate from one another or overlapping in time.In aspects, the vaccine comprises a unitary dose of between 0.1-3000 µg, including any value or range therebetween of polypeptide and/or nucleic acid of the instant disclosure.

The dosage of the vaccine, concentration of components therein and timing of administering the vaccine, which elicit a suitable immune response, can be determined by methods such as by antibody titrations of sera, e.g., by ELISA and/or seroneutralization assay analysis and/or by vaccination challenge evaluation as are known in the art.

In aspects, the vaccine comprises a T-cell epitope compound or composition of the instant disclosure in purified form, optionally in combination with any suitable excipient, carrier, adjuvant, and/or additional protein antigen.

ASFV vaccine constructs including a T-cell epitope compound or composition of the present disclosureupon administration to a subject may initiate a strong T-cell mediated immune response but may not always induce a humoral immune response. Therefore, in aspects of a vaccine,the vaccine contains a combination of a T-cell epitope compound or composition of the present disclosure together with either live attenuated virus (LAV, for example, live attenuated ASFV) or inactivated virus (for example, inactived ASFV). This vaccine composition (including both the T-cell epitope epitope compound or composition and an LAV or inactivated virus) upon administration to a subject may induce both cellular and humoral immune responses, thereby conferring comprehensive immunity against ASFV in the pigs.

Vaccines may comprise other ingredients, known per se by one of ordinary skill in the art, such as pharmaceutically acceptable carriers, excipients, diluents, adjuvants, freeze drying stabilizers, wetting or emulsifying agents, pH buffering agents, gelling or viscosity enhancing additives, and preservatives, depending on the route of administration.

Examples of pharmaceutically acceptable carriers, excipients or diluents include, but are not limited to demineralized or distilled water; saline solution; vegetable based oils such as peanut oil, arachis oil, safflower oil, olive oil, cottonseed oil, maize oil, sesame oil, or coconut oil; silicone oils, including polysiloxanes, such as methyl polysiloxane, phenyl polysiloxane and methylphenyl polysolpoxane; volatile silicones; mineral oils such as light liquid paraffin oil, or heavy liquid paraffin oil; squalene; cellulose derivatives such as methylcellulose, ethylcellulose, carboxymethylcellulose, carboxymethylcellulose sodium salt, or hydroxypropyl methylcellulose; lower alkanols, for example ethanol or isopropanol; lower aralkanols; lower polyalkylene glycols or lower alkylene glycols, for example polyethylene glycol, polypropylene glycol, ethylene glycol, propylene glycol, 1,3-butylene glycol or glycerin; fatty acid esters such as isopropyl palmitate, isopropyl myristate or ethyl oleate; polyvinylpyrrolidone; agar; carrageenan; gum tragacanth or gum acacia; and petroleum jelly. Typically, the carrier or carriers will form from 10% to 99.9% by weight of the vaccine composition and may be buffered by conventional methods using reagents known in the art, such as sodium hydrogen phosphate, sodium dihydrogen phosphate, potassium hydrogen phosphate, potassium dihydrogen phosphate, a mixture thereof, and the like.

Examples of adjuvants include, but are not limited to, oil in water emulsions, aluminum hydroxide (alum), immunostimulating complexes, non-ionic block polymers or copolymers, cytokines (like IL-1, IL-2, IL-7, IFN-α, IFN-β, IFN-γ, etc.), saponins, monophosphoryl lipid A (MLA), muramyl dipeptides (MDP), MCA, and the like. Other suitable adjuvants include, for example, aluminum potassium sulfate, heat-labile or heat-stable enterotoxin(s) isolated from Escherichia coli, cholera toxin or the B subunit thereof, diphtheria toxin, tetanus toxin, pertussis toxin, Freund’s incomplete or complete adjuvant, etc. Toxin-based adjuvants, such as diphtheria toxin, tetanus toxin and pertussis toxin may be inactivated prior to use, for example, by treatment with formaldehyde. Further adjuvants may include, but are not limited to, poly-ICLC, 1018 ISS, aluminum salts, Amplivax, AS 15, BCG, CP-870,893, CpG7909, CyaA, dSLIM, GM-CSF, IC30, IC31, Imiquimod, ImuFact IMP321, IS Patch, ISS, ISCOMATRTX, Juvlmmune, LipoVac, MF59, monophosphoryl lipid A, Montanide IMS 1312, Montanide ISA 206, Montanide ISA 50V, Montanide ISA-51, OK-432, OM-174, OM-197-MP-EC, ONTAK, PEPTEL, vector system, PLGA microparticles, resiquimod, SRL172, Virosomes and other Virus-like particles, YF-17D, VEGF trap, R848, beta-glucan, Pam3Cys, and Aquila’s QS21 stimulon.

Examples of freeze-drying stabilizer may be for example carbohydrates such as sorbitol, mannitol, starch, sucrose, dextran or glucose, proteins such as albumin or casein, and derivatives thereof.

Vaccines may additionally comprise at least one immunogen from at least one additional pathogen, e.g., a pig pathogen such as Actinobacillus pleuropneunomia; Adenovirus; Alphavirus such as Eastern equine encephalomyelitis viruses; Balantidium coli; Bordetella bronchiseptica; Brachyspira spp., preferably B. hyodyentheriae, B. pilosicoli, B. innocens, Brucella suis, preferably biovars 1, 2 and 3; Classical swine fever virus, Chlamydia and Chlamydophila spp., preferably C. pecorum and C. abortus; Clostridium spp., preferably Cl. difficile, Cl. perfringens types A, B and C, Cl. novyi, Cl. septicum, Cl. tetani; Digestive and respiratory Coronavirus; Cryptosporidium parvum; Eimeria spp.; Eperythrozoonis suis currently named Mycoplasma haemosuis; Erysipelothrix rhusiopathiae; Escherichia coli; Haemophilus parasuis, preferably subtypes 1, 7 and 14; Hemagglutinating encephalomyelitis virus; lsospora suis; Japanese Encephalitis virus; Lawsonia intracellulars; Leptospira spp., preferably Leptospira australis, Leptospira canicola, Leptospira grippotyphosa, Leptospira icterohaemorrhagicae, Leptospira interrogans, Leptospira Pomona and Leptospira tarassovi; Mannheimia haemolytica; Mycobacterium spp., preferably M. avium, M. intracellular and M. bovis: Mycoplasma hyponeumoniae; Parvovirus; Pasteurella multocida; Porcine circovirus; Porcine cytomegolovirus; Porcine parovirus, Porcine reproductive and respiratory syndrome virus: Pseudorabies virus; Rotavirus; Sagiyama virus; Salmonella spp., preferably S. thyhimurium and S. choleraesuis; Staphylococcus spp., preferably S. hyicus; Streptococcus spp., preferably Strep suis; Swine cytomegalovirus; Swine herpes virus; Swine influenza virus; Swinepox virus; Toxoplasma gondii; Vesicular stomatitis virus and virus of exanthema of swine; or other isolates and subtypes of porcine circovirus.

The vaccine compositions of the instant disclosure may be liquid formulations such as an aqueous solution, water-in-oil or oil-in-water emulsion, syrup, an elixir, a tincture, or a preparation for parenteral, subcutaneous, intradermal, intramuscular or intravenous administration (e.g., injectable administration), such as sterile suspensions or emulsions. Such formulations are known in the art and are typically prepared by dissolution of the antigen and other typical additives in the appropriate carrier or solvent systems. Liquid formulations also may include suspensions and emulsions that contain suspending or emulsifying agents.

The route of administration can be percutaneous, via mucosal administration, or via a parenteral route (intradermal, intramuscular, subcutaneous, intravenous, or intraperitoneal). Vaccine compositions according to the present disclosure may be administered alone, or can be co-administered or sequentially administered with other treatments or therapies. A vaccine of the present disclosure can conveniently be administered intranasally, transdermally (i.e., applied on or at the skin surface for systemic absorption), parenterally, ocularly, etc. The parenteral route of administration includes, but is not limited to, intramuscular, intravenous, intradermal, and intraperitoneal routes and the like. In aspects, vaccines of the present disclosure are administered intradermally, e.g., by using a commercial needle-free high-pressure device such as Pulse NeedleFree technology (Pulse 50TM Micro Dose Injection System, Pulse NeedleFree Systems; Lenexa, KS, USA).

The present disclosure also relates to methods of immunizing or inducing an immune response in non-human animals (e.g., pigs) comprising administering to said animal a polypeptide, concatemeric peptide, chimeric or fusion polypeptide, nucleic acid, cell, vector, pharmaceutical, or vaccine as described above.

The present disclosure also relates to methods of treating and/or preventing ASFV associated diseases in non-human animals (e.g., pigs) comprising administering to said animal a polypeptide, concatemeric peptide, chimeric or fusion polypeptide, nucleic acid, cell, vector, pharmaceutical, or vaccine as described above.

In one aspect, the vaccine compositions of the present disclosure are administered to a subject susceptible to or otherwise at risk for ASFV infection to enhance the subject own immune response capabilities. The subject to which the vaccine is administered is, in one embodiment, a pig. The animal may be susceptible to infection by ASFV or a closely related virus.

In aspects, the present disclosure includes multiple rounds of administration of the instantly-disclosed vaccine compositions. For example, the vaccine can be boosted at one, two, three, and/or four week intervals. Such are known in the art to improve or boost the immune system to improve protection against the targeted pathogen. Additionally, the present disclosure may also include assessing a subject’s immune system to determine if further administrations of the instantly-disclosed vaccine compositions is warranted. In some aspects, multiple administrations may include the development of a prime-boosting strategy of vaccination using the instantly-discloed vaccines (e.g.., polypeptide-based or nucleic acid-based as disclosed herein). Such may provide an opportunity to produce sequential immunogenic responses against ASFV and related diseases. In some aspects, the vaccine can be boosted at 1, 2, 3, 4, 5, or 6 week intervals. In some aspects, the vaccine is boosted at 2 week intervals. In some apsects, the vaccine is boosted at 3 week intervals. In some aspects, polypeptide-based vaccinations and nucleic acid-based (e.g., RNA or DNA) vaccinations can be achieved in an alternative manner to provide a regimen of immunization with the same immunogen presented in different fashions to the subject’s immune system.

Vaccines of the invention are preferably administered to pigs, adult pigs, and also young pigs, piglets or pregnant females, or to other types of non-human animals. Vaccination of pregnant females is particularly advantageous as it confers passive immunity to the newborns via the transmission of maternal antibodies. The pigs may be less than 7, 6, 5, 4, 3, 2 or 1 week old; 1 to 6 weeks old; 2 to 5 weeks old; or 3 to 4 weeks old. For instance, “test” animals may be administered the vaccine of the invention in order to evaluate the performance of the vaccine with a view to eventual use or development of a vaccine for pigs. Desirably, the vaccine is administered to a subject who has not yet been exposed to an ASFV virus. Preferably, the subject is a pig which is in need of vaccination against African Swine Fever.

The present invention also includes a combination vaccine, comprising vaccines of the invention and at least one immunogenic active component effective against another disease-causing organism in swine such as Actinobacillus pleuropneunomia; Adenovirus; Balantidium coli; Bordetella bronchiseptica; Brachyspira spp., preferably B. hyodyentheriae and B. pilosicoli, Brucella suis, preferably biovars 1, 2 and 3; Classical swine fever virus; Chlamydia and Chlamydophila spp., preferably C. pecorum and C. abortus; Clostridium spp., preferably Cl. difficile and Cl. perfringens; Porcine Respiratory Coronavirus; Cryptosporidium parvum; Eimeria spp.; Eperythrozoonis suis currently named Mycoplasma haemosuis; Erysipelothrix rhusiopathiae; Escherichia coli; Haemophilus parasuis; Hemagglutinating encephalomyelitis virus; Isospora suis; Lawsonia intracellulars; Leptospira spp., preferably Leptospira Pomona; Mannheimia haemolytica; Mycobacterium spp., preferably M. avium; Mycoplasma hyponeumoniae; Pasteurella multocida; Porcine circovirus; Porcine cytomegolovirus; Porcine parovirus; Porcine reproductive and respiratory syndrome virus; Pseudorabies virus; Rotavirus; Salmonella spp., preferably S. thyhimurium and S. choleraesuis; Staphylococcus spp., preferably S. hyicus; Streptococcus spp., preferably S. suis; Porcine Cytomegalovirus; Swine influenza virus; Swinepox virus; Toxoplasma gondii; Vesicular stomatitis virus and the virus of vesicular exanthema of swine; or other isolates and subtypes of ASFV.

The present disclosure also provides a container comprising an immunologically effective amount of a vaccine as described above. The present disclosure also provides vaccination kits comprising an optionally sterile container comprising an immunologically effective amount of the vaccine, means for administering the vaccine to animals, and optionally an instruction manual including information for the administration of the immunologically effective amount of the composition for treating and/or preventing ASFV associated diseases.

### Methods of Treatment

Stimulating T-cells with T-cell epitope compounds and compositions of the present disclosure can stimulate, induce, and/or expand corresponding naturally occurring immune response to ASFV and/or reated diseases caused by ASFV, including ASF, including CD4+ and CD8+ T-cell response, and in aspects results in increased secretion of one or more cytokines and chemokines. In aspects, T-cells activated by the T-cell epitope compounds and compositions of the present disclosure stimulate cell-mediated immunity against ASFV and/or reated diseases caused by ASFV, including ASF, in a subject.

In aspects, the present disclosure is directed to a method of stimulating, inducing, and/or expanding immunity against ASFV or related diseases in a subject in need thereof by administering to the subject a therapeutically effect amount of a T-cell epitope compound or composition of the present disclosure.

In aspects, the present disclosure is directed to a method of stimulating, inducing, and/or expanding an immune response, e.g., against ASFV infection (or a closely related virus) and/or related diseases caused by ASFV, in a subject in need thereof by administering to the subject a therapeutically effect amount of a T-cell epitope compound or composition of the present disclosure.

In aspects, the present disclosure is directed to a method of preventing, treating, or ameliorating a disease by ASFV infection (or a closely related virus), in a subject in need thereof by administering to the subject a therapeutically effect amount of a T-cell epitope compound or composition of the present disclosure

Further aspects and advantages of the invention are provided in the following section, which should be considered as illustrative only.

### Aspects

A 1st aspect is directed to a polypeptide consisting of an amino acid sequence selected from the group consisting of SEQ ID NOS: 1-65 and/or fragments and variants thereof, and optionally 1 to 12 additional amino acids distributed in any ratio on the N terminus and/or C-terminus of the polypeptide of SEQ ID NOS: 1-65.

A 2nd aspect is directed to a polypeptide consisting essentially of an amino acid sequence selected from the group consisting of SEQ ID NOS: 1-65 and/or fragments and variants thereof, and optionally 1 to 12 additional amino acids distributed in any ratio on the N terminus and/or C-terminus of the polypeptide of SEQ ID NOS: 1-65.

A 3rd aspect is directed to polypeptide comprising an amino acid sequence selected from the group consisting of SEQ ID NOS: 1-65 and/or fragments and variants thereof, and optionally 1 to 12 additional amino acids distributed in any ratio on the N terminus and/or C-terminus of the polypeptide of SEQ ID NOS: 1-65.

A 4th aspect is polypeptide according to apsects 1-3, wherein said variant or fragment of an amino acid sequence selected from the group consisting of SEQ ID NOS: 1-65 retains MHC binding propensity and TCR specificity, and/or retains anti-ASFV activity.

A 5th aspect is directed to a polypeptide consisting of an amino acid sequence having at least 75%, 80%, 85%, 90%, or 95% homology to any one of SEQ ID NOS: 1-65, wherein said polypeptide retains MHC binding propensity and the same TCR specificity, and/or retains ASFV activity.

A 6th aspect is directed to a polypeptide consisting essentially of an amino acid sequence having at least 75%, 80%, 85%, 90%, or 95% homology to any one of SEQ ID NOS: 1-65, wherein said polypeptide retains MHC binding propensity and the same TCR specificity, and/or retains ASFV activity.

A 7th aspect is directed to a polypeptide comprising an amino acid sequence having at least 75%, 80%, 85%, 90%, or 95% homology to any one of SEQ ID NOS: 1-65, wherein said polypeptide retains MHC binding propensity and the same TCR specificity, and/or retains ASFV activity.

An 8th aspect is directed to a polypeptide comprising an amino acid sequence selected from the group consisting of SEQ ID NOS: 66-67 and 70-71, and fragments or variants thereof.

A 9th aspect is directed to a polypeptide according to aspet 8, wherein said fragment or variant of a polypeptide comprising an amino acid sequence selected from the group consisting of SEQ ID NOS: 66-67 and 70-71 retains ASFV activity.

A 10th aspect is directed to a polypeptide comprising an amino acid sequence having at least 60%, 70%, 80%, 90%, or 95% homology to SEQ ID NO 66, wherein said polypeptide retains ASFV activity.

An 11th aspect is directed to a polypeptide comprising an amino acid sequence having at least 60%, 70%, 80%, 90%, or 95% homology to SEQ ID NO 67, wherein said polypeptide retains ASFV activity.

A 12th aspect is directed to a polypeptide comprising an amino acid sequence having at least 60%, 70%, 80%, 90%, or 95% homology to SEQ ID NO 70, wherein said polypeptide retains ASFV activity.

An 13th aspect is directed to a polypeptide comprising an amino acid sequence having at least 60%, 70%, 80%, 90%, or 95% homology to SEQ ID NO 71, wherein said polypeptide retains ASFV activity.

A 14th aspect is directed to a nucleic acid encoding a polypeptide consisting of an amino acid sequence selected from the group consisting of SEQ ID NOS: 1-65 and/or fragments and variants thereof, and optionally 1 to 12 additional amino acids distributed in any ratio on the N terminus and/or C-terminus of the polypeptide of SEQ ID NOS: 1-65.

A 15th aspect is directed to a nucleic acid encoding a polypeptide consisting essentially of an amino acid sequence selected from the group consisting of SEQ ID NOS: 1-65 and/or fragments and variants thereof, and optionally 1 to 12 additional amino acids distributed in any ratio on the N terminus and/or C-terminus of the polypeptide of SEQ ID NOS: 1-65.

A 16th aspect is directed to a nucleic acid encoding a polypeptide comprising an amino acid sequence selected from the group consisting of SEQ ID NOS: 1-65 and/or fragments and variants thereof, and optionally 1 to 12 additional amino acids distributed in any ratio on the N terminus and/or C-terminus of the polypeptide of SEQ ID NOS: 1-65.

A 17th aspect is directed to a nucleic acid encoding a polypeptide comprising an amino acid selected from the group consisting of SEQ ID NOS: 66-67 and 70-71 and/or fragments and variants thereof.

A 18th aspect is directed to a nucleic acid consisting of a sequence selected from the group consisting of SEQ ID NOS: 68-69, 72-77 and fragments or variants thereof.

A 19th aspect is directed to a nucleic acid consisting essentially of a sequence selected from the group consisting of SEQ ID NOS: 68-69, 72-77 and fragments or variants thereof.

A 20th aspect is directed to a nucleic acid comprising a sequence selected from the group consisting of SEQ ID NOS: 68-69, 72-77 and fragments or variants thereof.

A 21st aspect is directed to a nucleic acid comprising a sequence selected from the group consisting of SEQ ID NOS: 68, 72, 74, and 76 and fragments or variants thereof.

A 22nd aspect is directed to a nucleic acid of any one of aspects 14-16, wherein said fragment or variant of the nucleic acid encoding a polypeptide comprising an amino acid sequence selected from the group consisting of SEQ ID NOS: 1-65 encodes a polypeptide that retains ASFV activity.

A 23rd aspect is directed to a nucleic acid of aspect 17, wherein said fragment or variant of the nucleic acid encoding a polypeptide comprising an amino acid selected from the group consisting of SEQ ID NOS: 66-67 and 70-71 encodes a polypeptide that retains ASFV activity.

A 24th aspect is directed to a nucleic acid of any one of aspects 18-20, wherein said fragment or variant of the nucleic acid encoding a polypeptide comprising a sequence selected from the group consisting of SEQ ID NOS: 68-69, 72-77 encodes a polypeptide that retains anti-ASFV activity.

A 25th aspect is directed to a nucleic acid of aspect 21, wherein said fragment or variant of the nucleic acid encoding a polypeptide comprising a sequence selected from the group consisting of SEQ ID NOS: 68, 72, 74, and 76 encodes a polypeptide that retains anti-ASFV activity.

A 26th aspect is directed to a nucleic acid comprising a sequence with at least 60%, 70%, 80%, 90%, or 95% homology to SEQ ID NO 68, provided said polypeptide encoded by said nucleic retains anti-ASFV activity.

A 27th aspect is directed to a nucleic acid comprising a sequence with at least 60%, 70%, 80%, 90%, or 95% homology to SEQ ID NO 69, provided said polypeptide encoded by said nucleic retains anti-ASFV activity.

A 28th aspect is directed to a nucleic acid comprising a sequence with at least 60%, 70%, 80%, 90%, or 95% homology to SEQ ID NO 72, provided said polypeptide encoded by said nucleic retains anti-ASFV activity.

A 29th aspect is directed to a nucleic acid comprising a sequence with at least 60%, 70%, 80%, 90%, or 95% homology to SEQ ID NO 73, provided said polypeptide encoded by said nucleic retains anti-ASFV activity.

A 30th aspect is directed to a nucleic acid comprising a sequence with at least 60%, 70%, 80%, 90%, or 95% homology to SEQ ID NO 74, provided said polypeptide encoded by said nucleic retains anti-ASFV activity.

A 31st aspect is directed to a nucleic acid comprising a sequence with at least 60%, 70%, 80%, 90%, or 95% homology to SEQ ID NO 75, provided said polypeptide encoded by said nucleic retains anti-ASFV activity.

A 32nd aspect is directed to a nucleic acid comprising a sequence with at least 60%, 70%, 80%, 90%, or 95% homology to SEQ ID NO 76, provided said polypeptide encoded by said nucleic retains anti-ASFV activity.

A 33rd aspect is directed to a nucleic acid comprising a sequence with at least 60%, 70%, 80%, 90%, or 95% homology to SEQ ID NO 77, provided said polypeptide encoded by said nucleic retains anti-ASFV activity.

A 34^{th} aspect is directed to a plasmid comprising a nucleic acid of any one of aspects 14-17, 21-23, 25-26, 28, 30, and 32.

A 35th aspect is directed to a vector comprising a nucleic acid according to any one of any one of apsects 14-17, 21-23, 25-26, 28, 30, and 32.

A 36th aspect is directed to a pharmaceutical composition comprising a polypeptide according to any one of aspects 1-15 and a pharmaceutically-acceptable carrier and/or excipient.

A 37th aspect is directed to a pharmaceutical composition comprising a nucleic acid according to any one of aspects 16-33 and a pharmaceutically-acceptable carrier and/or excipient.

A 38th aspect is directed to a pharmaceutical composition comprising a plasmid according to aspect 34 and a pharmaceutically-acceptable carrier and/or excipient.

A 39th aspect is directed to a pharmaceutical composition comprising a vector according to aspect 35 and a pharmaceutically-acceptable carrier and/or excipient.

A 40th aspect is directed to a vaccine comprising a polypeptide according to any one of aspects 1-15 and a pharmaceutically-acceptable excipient, carrier, and/or adjuvant.

A 41st aspect is directed to a vaccine comprising a nucleic acid according to any one of aspects 16-33 and a pharmaceutically-acceptable excipient, carrier, and/or adjuvant.

A 42nd aspect is directed to a vaccine comprising a plasmid according to aspect 34 and a pharmaceutically-acceptable excipient, carrier, and/or adjuvant.

A 43^{rd} aspect is directed to a vaccine comprising a vector according to aspect 35 and a pharmaceutically-acceptable excipient, carrier, and/or adjuvant.

A 44th aspect is directed to a method for inducing immunity against ASFV in a subject in need thereof, the method comprising administering to the subject a therapeutically effective amount of a polypeptide according to any one of aspects 1-15.

A 45th aspect is directed to a method for inducing immunity against ASFV in a subject in need thereof, the method comprising administering to the subject a therapeutically effective amount of a nucleic acid according to any one of aspects 16-33.

A 46th aspect is directed to a method for inducing immunity against ASFV in a subject in need thereof, the method comprising administering to the subject a therapeutically effective amount of a plasmid according to aspect 34.

A 47th aspect is directed to a method for inducing immunity against ASFV in a subject in need thereof, the method comprising administering to the subject a therapeutically effective amount of a vector according to aspect 35.

A 48th aspect is directed to a method for inducing immunity against ASFV in a subject in need thereof, the method comprising administering to the subject a therapeutically effective amount of a pharmaceutical composition according to any one of aspects 36-39.

A 49th aspect is directed to a method for inducing immunity against ASFV in a subject in need thereof, the method comprising administering to the subject a therapeutically effective amount of a vaccine composition according to any one of aspects 40-43.

A 50th aspect is directed to a method according to any one of aspects 44-49, wherein the step of administration additionally includes administration of an African Swine Fever Virus, wherein the virus is a live attenuated virus or inactivated virus.

A 51st aspect is directed to a method for inducing an immune response against ASFV infection and/or related diseases cause by ASFV infection in a subject in need thereof, the method comprising administering to the subject a therapeutically effective amount of one or more of a polypeptide according to any one of aspects 1-15.

A 52nd aspect is directed to a method for inducing an immune response against ASFV infection and/or related diseases cause by ASFV infection in a subject in need thereof, the method comprising administering to the subject a therapeutically effective amount of a nucleic acid according to any one of aspects 16-33.

A 53rd aspect is directed to a method for inducing an immune response against ASFV infection and/or related diseases cause by ASFV infection in a subject in need thereof, the method comprising administering to the subject a therapeutically effective amount of a plasmid according to aspect 34.

A 54th aspect is directed to a method for inducing an immune response against ASFV infection and/or related diseases cause by ASFV infection in a subject in need thereof, the method comprising administering to the subject a therapeutically effective amount of a vector according to aspect 35.

A 55th aspect is directed to a method for inducing an immune response against ASFV infection and/or related diseases cause by ASFV infection in a subject in need thereof, the method comprising administering to the subject a therapeutically effective amount of a pharmaceutical composition according to any one of aspects 36-39.

A 56th aspect is directed to a method for inducing an immune response against ASFV infection and/or related diseases cause by ASFV infection in a subject in need thereof, the method comprising administering to the subject a therapeutically effective amount of a vaccine composition according to any one of aspects 40-43.

A 57th aspect is directed to a method according to any one of aspects 51-56, wherein the step of administration additionally includes administration of an African Swine Fever Virus, wherein the virus is a live attenuated virus or inactivated virus.

A 58^{th} aspect is directed to a chimeric or fusion polypeptide comprising a polypeptide of any one of aspects 1-15, wherein said polypeptide is joined, linked, or inserted into a heterologous polypeptide.

### EXAMPLES

The examples that follow are not to be construed as limiting the scope of the invention in any manner. In light of the present disclosure, numerous embodiments within the scope of the claims will be apparent to those of ordinary skill in the art.

### In-Silico Identification of Potential Epitopes for SLA

T-cells specifically recognize epitopes presented by cells in the context of MHC (Major Histocompatibility Complex) Class I and II molecules. These T-cell epitopes can be represented as linear sequences comprising 7 to 30 contiguous amino acids that fit into the MHC Class I or II binding groove. A number of computer algorithms have been developed and used for detecting Class I and II epitopes within protein molecules of various origins (De Groot AS et al., (1997), AIDS Res Hum Retroviruses,13(7):539-41; Schafer JR et al., (1998), Vaccine, 16(19): 1880-4; De Groot AS et al., (2001), Vaccine, 19(31):4385-95; De Groot AS et al., (2003), Vaccine, 21(27-30):4486-504). These “in silico” predictions of T-cell epitopes have been successfully applied to the design of vaccines and the de-immunization of therapeutic proteins, i.e. antibody-based drugs, Fc fusion proteins, anticoagulants, blood factors, bone morphogenetic proteins, engineered protein scaffolds, enzymes, growth factors, hormones, interferons, interleukins, and thrombolytics (Dimitrov DS, (2012), Methods Mol Biol, 899:1-26).

The Conservatrix system (EpiVax, Providence, Rhode Island) is an algorithm useful for identifying 9-mer polypeptide sequences from a larger set of data. The Conservatrix system parses input sequences into 9-mer sequences that are conserved amongst multiple inputted whole sequences, such as multiple strains of the same pathogen, for even the most mutable of potential vaccine targets. These 9-mer sequences may be searched for identically matched 9-mer sequences across data sets.

The PigMatrix™ system (EpiVax, Providence, Rhode Island) is a set of predictive algorithms encoded into computer programs useful for predicting class I and class II SLA ligands and T-cell epitopes. The PigMatrix™ system uses matrices in order to model the interaction between specific amino acids and binding positions within the SLA molecule. In order to identify putative epitopes resident within any given input protein, the PigMatrix™ System first parses the input protein into a set of overlapping n-mer frames (n=length of amino acids of epitope peptide being screen; e.g., n=9) where each frame overlaps the last by n-1 amino acids. Each frame is then scored for predicted affinity to one or more common alleles of the swine SLA molecule. Briefly, for any given n-mer peptide specific amino acid codes (one for each of 20 naturally occurring amino acids) and relative binding positions (1 to n) are used to select coefficients from the predictive matrix. Individual coefficients are derived using a proprietary method similar to, but not identical to, the pocket profile method first developed by Stumiolo (Sturniolo T et al., 1999, Nat Biotechnol, 17(6):555-61). Individual coefficients are then summed to produce a raw score. PigMatrix™ raw scores are then normalized with respect to a score distribution derived from a very large set of randomly generated peptide sequences. The resulting “Z” scores are normally distributed and directly comparable across alleles. It was determined that any peptide scoring above 1.64 on the PigMatrix™ “Z” scale (approximately the top 5% of any given peptide set) has a significant chance of binding to the MHC molecule for which it was predicted. Peptides scoring above 2.32 on the scale (the top 1%) are extremely likely to bind.

The JanusMatrix system (EpiVax, Providence, Rhode Island) is useful for screening peptide sequences for cross-conservation with a host proteome. JanusMatrix is an algorithm that predicts the potential for cross-reactivity between peptide clusters and the host genome or proteome, based on conservation of TCR-facing residues in their putative MHC ligands. The JanusMatrix algorithm first considers all the predicted epitopes contained within a given protein sequence and divides each predicted epitope into its constituent agretope and epitope. Each sequence is then screened against a database of host proteins. Peptides with a compatible MHC-facing agretope (i.e., the agretopes of both the input peptide and its host counterparty are predicted to bind the same MHC allele) and exactly the same TCR-facing epitope are returned. The JanusMatrix Homology Score suggests a bias towards immune tolerance. In the case of a therapeutic protein, cross-conservation between autologous human epitopes and epitopes in the therapeutic may increase the likelihood that such a candidate will be tolerated by the human immune system. In the case of a vaccine, cross-conservation between human epitopes and the antigenic epitopes may indicate that such a candidate utilizes immune camouflage, thereby evading the immune response and making for an ineffective vaccine. When the host is, for example, a pig, the peptide clusters are screened against swine genomes and proteomes, based on conservation of TCR-facing residues in their putative SLA ligands. The peptides are then scored using the JanusMatrix Swine Homology Score. Peptides with a JanusMatrix Swine Homology Score below 2.5 indicate low tolerogenicity potential and may be useful for vaccines.The EpiAssembler system is useful for assembling overlapping epitopes to Immunogenic Consensus Sequences (ICS). EpiAssembler is an algorithm that optimizes the balance between pathogen and population coverage. EpiAssembler uses the information from the sequences produced by Conservatrix and EpiMatrix to form highly immunogenic consensus sequences.

The VaccineCAD system is useful for arranging potential epitopic vaccine candidates into a string to avoid creation of novel epitopes upon joining of the vaccine candidate sequences. Specifically, VaccineCAD designs potential vaccine candidates into a string-of-beads vaccine while minimizing any deleterious, non-specific junctional epitopes that may appear in the joining process. VaccineCAD may use PigMatrix to predict junctional epitopes.

### Example 1: ASFV Proteome Analysis

Complete proteomes of 21 ASFV strains from Europe, Africa, and Asia were downloaded from GenBank. The downloaded proteomes were analyzed to identify putative SLA Class I and Class II T-cell epitopes. The analyzed set included seven genotype I, seven genotype II, six genotype IX, and one genotype X ASFV strains collected from 1960 to 2018. In total, 3587 proteins were screened; the number of proteins per virus ranged from 152 to 196. Table 1 summarizes the analysis performed on the proteomes from the set of ASFV strains.

TABLE 1

The analysis of the proteomes of the 21 downloaded ASFV strains.

| Strain | Genome accession | Country | Collection year | # of Proteins | Publication | PubMed | Genotype |
|---|---|---|---|---|---|---|---|
| L60 | KM262844 | Portugal | 1960 | 163 | Gomez-Villamandos J | 10.1016/j.virusres.2013.01.017 | I |
| NHV | KM262845 | Portugal | 1968 | 158 | Gbmez-Villamandos J | 10.1016/j.virusres.2013.01.017 | I |
| BA71 | KP055815 | Spain | 1971 | 161 | Rodriguez J 2015 | 10.1371/journal.pone.0142889 | I |
| BA71V | U18466 | | | 152 | Rodriguez J 2015 | 10.1371/journal.pone.0142889 | I |
| E75 | FN557520 | Spain | 1975 | 163 | de Villers E 2010 | 10.1016/j.viro1.2010.01.019 | I |
| OURT88_3 | AM712240 | Portugal | 1988 | 157 | Chapman D 2008 | 10.1099/vir.0.83343-0 | I |
| Benin97_1 | AM712239 | Benin | 1997 | 156 | Chapman D 2008 | 10.1099/vir.0.83343-0 | I |
| Ken05/Tk1 | KM111294 | Kenya | 2005 | 168 | Bishop R 2015 | 10.1007/s 11262-014-1156-7 | X |
| Ken06.Bus | KM111295 | Kenya | 2006 | 161 | Bishop R 2015 | 10.1007/s 11262-014-1156-7 | IX |
| Georgia 2007/1 | FR682468 | Georgia | 2007 | 188 | Chapman D 2011 | 10.3201/eid1704.101283 | II |
| Georgia 2008/1 | MH910495 | Georgia | 2008 | 179 | Farlow J 2018 | 10.1186/s 12985-018-1099-z | II |
| Estonia 2014 | LS478113 | Estonia | 2014 | 173 | Zani L 2018 | 10.1038/s41598-018-24740-1 | II |
| R8 | MH025916 | Uganda | 2015 | 173 | Masembe C 2018 | 10.1128/MRA.01018-18 | IX |
| R7 | MH025917 | Uganda | 2015 | 174 | Masembe C 2018 | 10.1128/MRA.01018-18 | IX |
| R25 | MH025918 | Uganda | 2015 | 172 | Masembe C 2018 | 10.1128/MRA.01018-18 | IX |
| N10 | MH025919 | Uganda | 2015 | 171 | Masembe C 2018 | 10.1128/MRA.01018-18 | IX |
| R35 | MH025920 | Uganda | 2015 | 173 | Masembe C 2018 | 10.1128/MRA.01018-18 | IX |
| Belgium 2018/1 | LR536725 | Belgium | 2018 | 196 | Direct submission | NA | II |
| China/2018/AnhuiXCGQ | MK128995 | China | 2018 | 179 | Bao J 2019 | 10.1111/tbed.13124 | II |
| DB/LN/2018 | MK333181 | China | 2018 | 185 | Wen X 2019 | 10.1080/22221751.2019.1565915 | II |
| Pig/HLJ/2018 | MK333180 | China | 2018 | 185 | Wen X 2019 | 10.1080/22221751.2019.1565915 | II |

### Example 2: Epitope Selection and Design of Asf Vaccine

Complete proteomes of 21 ASFV strains were downloaded from GenBank, as described in Example 1. Proteins were loaded to iVAX and parsed into 9-mers overlapping by eight amino acids using the Conservatrix algorithm. Nine-mer sequences were searched for identically matched 9-mers among ASFV strains. Nine-mers conserved in the 21 ASFV strains were scored for binding potential against a panel of 13 SLA class I (SLA-I*0101, 1*0401, 1*0801, 1*1201, 1*1301, 2*0101, 2*0401, 2*0501, 2*1201, 3*0401, 3*0501, 3*0601, 3*0701) and 8 class II (SLA-DRB1*0101, 0201, 0401, 0402, 0601, 0602, 0701, 1001) alleles using PigMatrix. PigMatrix raw scores were standardized to Z-scores to compare potential epitopes across multiple SLA alleles. Peptides with Z-scores above 1.64 (the top 5% of any given sample of 9-mers) were identified as likely to be SLA ligandsFor class I, the 13 SLA alleles were grouped based on their binding preferences and five 9-mers with the highest binding likelihood per each of the eight defined groups were selected. For class II, highly conserved and epitope-dense protein regions (T-cell epitope clusters of at least two highly conserved 9-mer frames predicted to bind to four or more SLA alleles) were selected. No more than two putative T-cell epitopes from the same source protein were further analyzed. Putative T-cell epitopes were also screened for cross-conservation with the pig proteome using JanusMatrix. In aspects, epitopes with JanusMatrix Swine Homology Scores below 2.5 (low tolerogenicity potential), or below 2.0, were selected.

A total of 40 class I and 26 class II peptides were selected for inclusion in the ASF vaccine constructs, following immunoinformatic predictions. Selection was based on high conservation among ASFV strains, high binding likelihood to 13 SLA class I and 8 class II alleles, and low tolerogenicity potential. Putative class I epitopes were conserved in all the analyzed ASFV strains, were in the top 1% of predicted ligands, and had Janus Swine Homology Scores below 2, with exception of three 9-mers with scores below 2.5. Putative class II epitopes had at least two 9-mer frames conserved in all the ASFV strains, were predicted to bind to four or more SLA alleles, and had Janus Swine Homology Scores below 2. The selected 9-mer epitopes for SLA Class I include sequences SEQ. ID NO 1-39. The selected epitope clusters for SLA Class II include sequences SEQ ID NO 40-65.

Predicted epitope sequences were concatenated using EpiAssembler to form two multi-epitope pseudo-proteins (one for SLA class I and one for class II epitopes). The multi-epitope pseudo-protein for SLA Class I comprises the sequence of SEQ. ID NO 66; the multi-epitope pseudo-protein for SLA Class II comprises the sequence of SEQ. ID NO 70. Two vaccine constructs predicted to have no junctional epitopes were designed. VaccineCAD was then used to rearrange the peptides to avoid creation of novel epitopes at peptide junctions. VaccineCAD used PigMatrix to predict junctional epitopes. Where reordering did not sufficiently reduce the potential for junctional immunogenicity, a cleavage promoting motif (‘AAY’) for the class I-restricted construct or a binding inhibiting ‘breaker’ sequence (‘GPGPG’) for the class II-restricted construct, was introduced between peptides to optimize epitope processing. The class I construct sequence contained 40 putative epitopes and 16 AAY cleavage motifs. The class II construct sequence contained 26 putative T-cell epitope clusters; breaker sequences were not required to reduce junctional immunogenicity potential. These post-VaccineCAD, optimized multi-epitope constructs are represented by sequences SEQ ID NO: 67 for SLA class I and SEQ ID NO: 71 for SLA class II. The total lengths of the Class I and II constructs were 399 and 511 amino acids, respectively.

The two multi-epitope pseudo-proteins were back-translated to DNA. DNA back-translations of the pseudo-proteins are given by SEQ. ID NO 68 for class I and SEQ. ID NO 72 for class II. Genes were codon-optimized and synthesized by GeneArt (Life Technologies, NY, USA). Tandem stop codons were incorporated downstream of the epitope sequences.

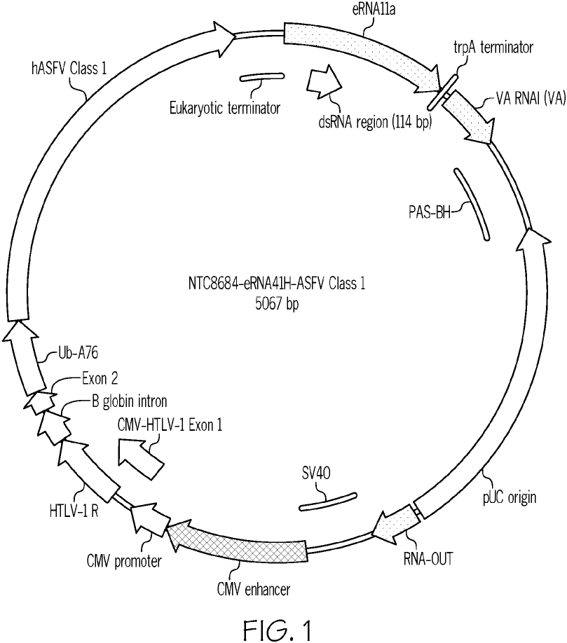

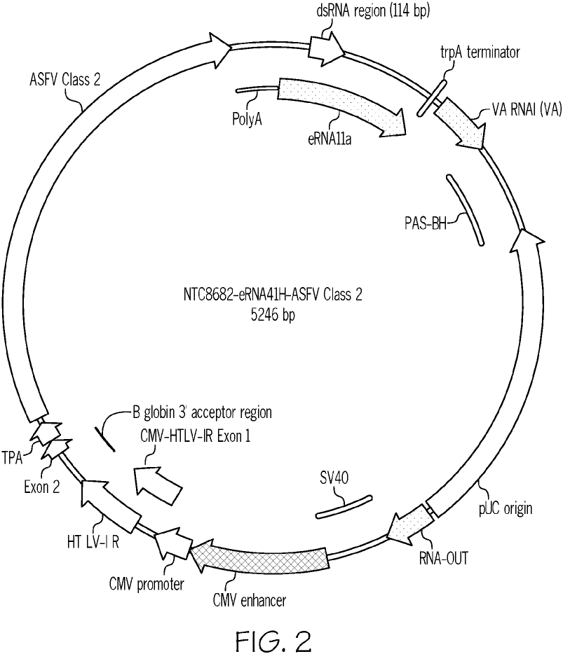

For the class I vaccine construct, class I genes were subcloned at predefined flanking restriction sites downstream a destabilizing UbiquitinA76 tag (UbA76) in pNTC8684-eRNA41H for proteasome targeting. For the class II vaccine construct, class II genes were subcloned at predefined flanking restriction sites downstream a tissue plasminogen activator (TPA) leader sequence in pNTC8682-eRNA41H (Nature Technology Corporation, NE, USA) for secretory pathway targeting. High-purity plasmids for immunizations were prepared by Nature Technology Corporation, Inc. at research grade. Each plasmid underwent quality control testing including spectrophotometric concentration and A260/A280 ratio determination (1.97), restriction digest analysis to assure the presence of the multi-epitope genes, agarose gel electrophoresis determination of residual host RNA and DNA (none detected), and quantitative endotoxin testing (<2.0 EU/mg). The SLA class I plasmid that was generated is illustrated in FIG. 1, and its sequence is comprised of SEQ. ID NO 69. The SLA class II plasmid that was generated is illustrated in FIG. 2, and its sequence is comprised of SEQ. ID NO 73.

### Example 3: Immunogenicity Study to Assess Immunogen Expression

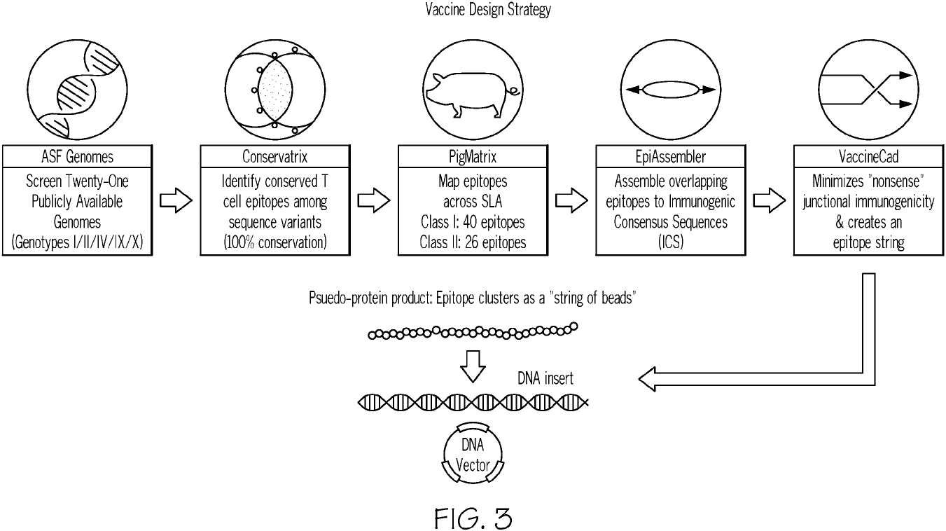

As described above, using the iVax computational vaccine design platform, a T-cell-directed ASF vaccine was developed that was composed of swine MHC class I and class II epitopes conserved across 21 European, Asian and African isolates covering genotypes I, II, IX, and X, and multi-epitope genes encoding class I and class II epitopes separately were each subcloned into plasmids to produce a DNA vaccine (FIG. 3). High-purity plasmids for immunizations were prepared at research grade (Nature Technology; Lincoln, Nebraska, USA).

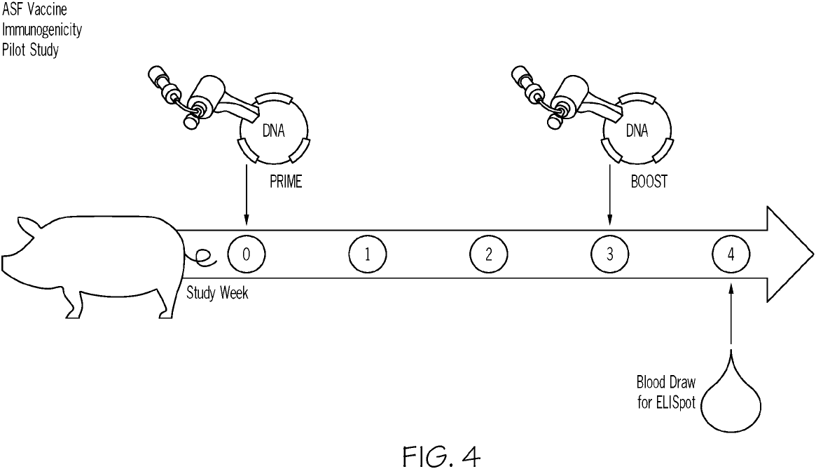

A pilot immunogenicity study was performed to assess immunogen expression in vivo (FIG. 4). Four-week old pigs (N=3) were primed and boosted three weeks later with plasmid DNA vaccine delivered intradermally using Pulse NeedleFree technology. A matching group of pigs received empty plasmid (no epitopes) as a control for epitope-specific immune responses. The plasmid DNA (pDNA) vaccine and control vaccine were administered intradermally in the neck using a commercial needle-free high-pressure device (Pulse 50TM Micro Dose Injection System, Pulse NeedleFree Systems; Lenexa, KS, USA).

One week later, blood was collected and peripheral blood leukocytes isolated. To obtain peripheral blood mononuclear cells (PBMCs), 8-10 mL blood was collected from each pig using BD Vacutainer_CPTTM cell preparation tubes with sodium citrate (Becton, Dickinson and Company). Within 2 h of blood collection, the tubes were centrifuged at 1800 g for 20 min at room temperature. The buffy coat was collected and resuspended in PBS. Cells were washed and centrifuged at 500 g for 5 min at 4° C., the supernatant was discarded, and the pellet was used immediately for the ELISpot assay.

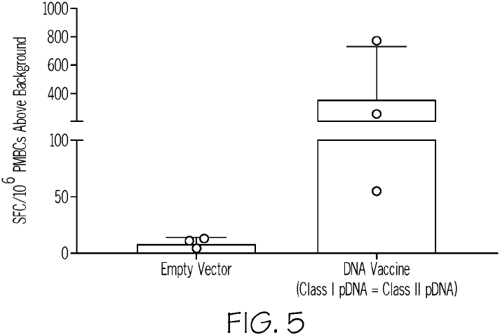

For the ELISpot assay, the peptides were selected to match the epitopes presented in the pDNA vaccine. For control purposes, PBMCs were stimulated with pokeweed mitogen (PWM). The cells were then incubated for 36 h at 37° C. in a 5% CO2 incubator. Subsequently, the ELISPOT assay was performed according to the manufacturer’s instructions. Blue-black colored precipitate spots corresponding to activated IFNy secreting cells were counted with an ELISPOT reader (ImmunoSpot ELISPOT analyzer, Cellular Technology Limited, Cleveland, OH, USA). Recall responses to vaccine epitopes was measured by IFNγ ELISpot assay. Epitope-specific IFNγ responses were detected in all three pigs that received the ASF DNA vaccine (FIG. 5). No control pigs responded. PigMatrix identified swine T-cell epitopes that stimulate strong ASF-specific IFNy responses in pigs that received an epitope-string DNA vaccine. The results suggest that this ASF plasmid DNA vaccine may be effective against ASF virus challenge, and in aspects may be used in combination with an inactivated or live attenuated virus vaccine in a heterologous prime-boost regimen.

### Example 4: Immunogenicity Study to Measure Immune Response

As described above, using the iVax computational vaccine design platform, a T-cell -directed ASF vaccine was developed that was composed of swine MHC class I and class II epitopes conserved across 21 European, Asian and African isolates covering genotypes I, II, IX, and X, and multi-epitope genes encoding class I and class II epitopes separately were each subcloned into plasmids to produce a DNA vaccine (FIG. 3). High-purity plasmids for immunizations were prepared at research grade (Nature Technology; Lincoln, Nebraska, USA).

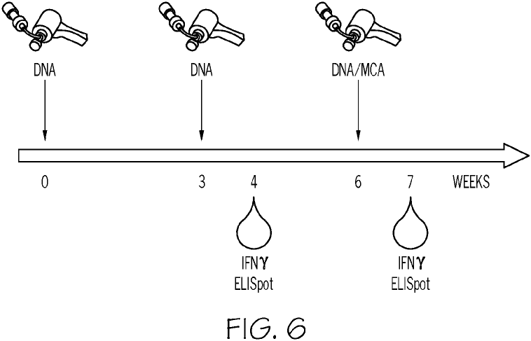

A pilot immunogenicity study was performed to assess immunogen expression in vivo (FIG. 6). Four-week old pigs were primed and boosted three weeks and six weeks later with plasmid DNA (pDNA) vaccine delivered intradermally using Pulse NeedleFree technology (Pulse 50TM Micro Dose Injection System, Pulse NeedleFree Systems; Lenexa, KS, USA). The pDNA vaccine was a 0.5 mL dose containing class I pDNA and class II pDNA at 133 µg total plasmid dose (1:1 ratio of (class I pDNA):(class II pDNA)). The six-week boost vaccine was a 0.5 mL dose containing class I pDNA and class II pDNA at 133 µg total plasmid dose (1:1 ratio of (class |pDNA):(class II pDNA)) and an MCA (PGT) adjuvant dose at 798 µg (with the pDNA:MCA at a 1:6 w/w ratio). A matching group of pigs received empty plasmid (no epitopes) as a control for epitope-specific immune responses. The pDNA vaccine and control vaccine were administered intradermally in the neck using a commercial needle-free high-pressure device (Pulse 50TM Micro Dose Injection System, Pulse NeedleFree Systems; Lenexa, KS, USA). The boost at six weeks later included an MCA adjuvant.

One week after each boost, blood was collected and peripheral blood leukocytes isolated. To obtain peripheral blood mononuclear cells (PBMCs), 8-10 mL blood was collected from each pig using BD Vacutainer_ CPTTM cell preparation tubes with sodium citrate (Becton, Dickinson and Company). Within 2 h of blood collection, the tubes were centrifuged at 1800 g for 20 min at room temperature. The buffy coat was collected and resuspended in PBS. Cells were washed and centrifuged at 500 g for 5 min at 4° C., the supernatant was discarded, and the pellet was used immediately for the ELISpot assay.

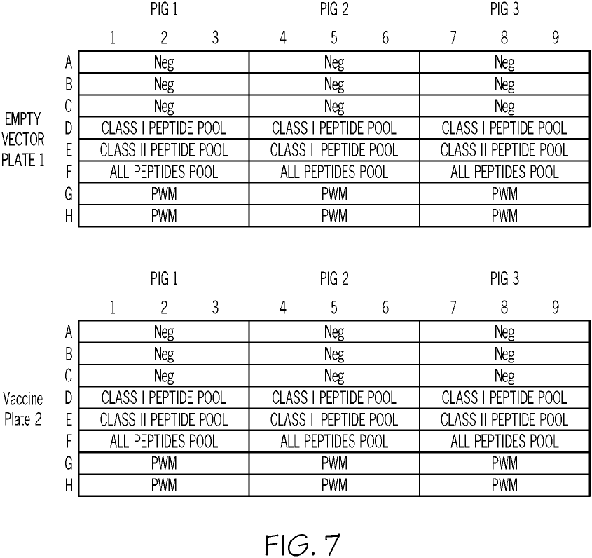

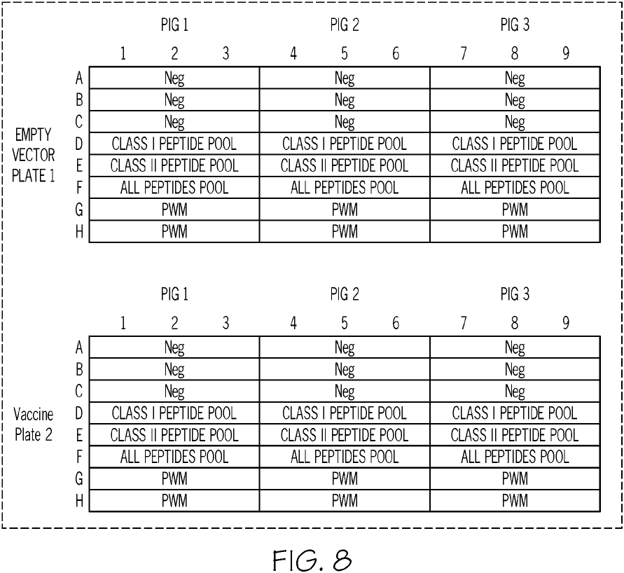

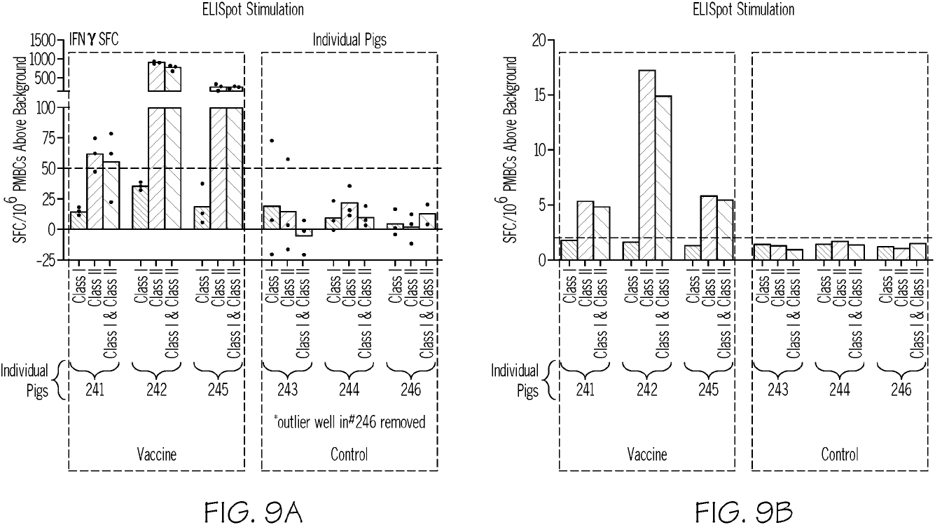

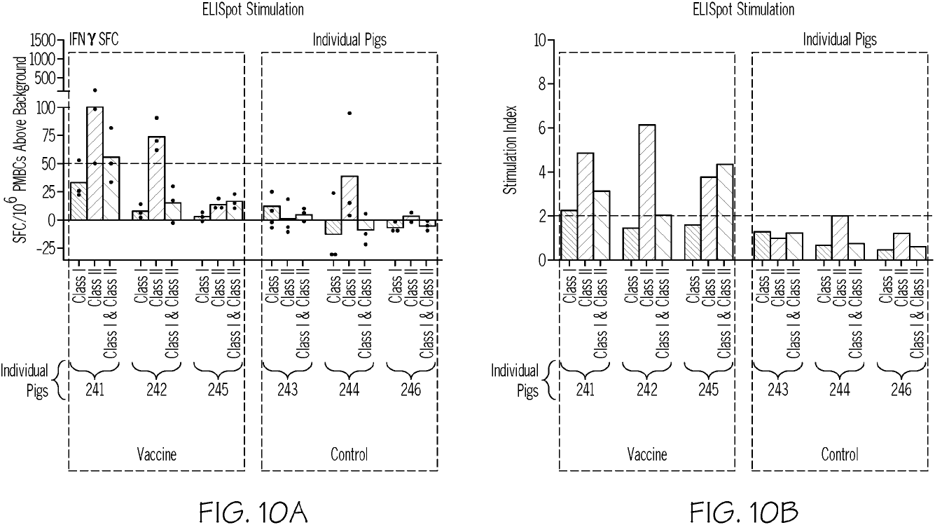

For the ELISpot assay, the peptides were selected to match the epitopes presented in the pDNA vaccine. For control purposes, PBMCs were stimulated with pokeweed mitogen (PWM). The cells were then incubated for 36 h at 37° C. in a 5% CO2 incubator. Subsequently, the ELISPOT assay was performed according to the manufacturer’s instructions. Blue-black colored precipitate spots corresponding to activated IFNy secreting cells were counted with an ELISPOT reader (ImmunoSpot ELISPOT analyzer, Cellular Technology Limited, Cleveland, OH, USA). Recall responses to vaccine epitopes was measured by IFNγ ELISpot assay for the week 3 boost (blood collected at week 4) (FIG. 7) and the week 6 boost (blood collected at week 7) (FIG. 8). For the week 3 boost, epitope-specific IFNy responses were detected in all three pigs that received the ASF DNA vaccine, indicating that the ASF DNA vaccine prime-boost stimulates a strong immune response (FIG. 9A and FIG. 9B). For the week 6 boost that included that MCA adjuvant, epitope-specific IFNγ responses were detected in two out of the three pigs that received the ASF DNA vaccine (FIG. 10A and FIG. 10B). These results indicate a strong ASF epitope-specific IFNγ response in immunized pigs, which is clear evidence that the ASF T-cell epitope vaccine is expressed.

### SEQUENCE LISTING

### Number of SEQ ID NOS: 73

SEQ ID NO 1
LENGTH: 9
TYPE: Protein
OTHER INFORMATION: Class I 9-mer C962R_ 607

SEQUENCE: 1

Glu Leu Asp Ala Arg Leu Trp Ile Met

SEQ ID NO 2
LENGTH: 9
TYPE: Protein
OTHER INFORMATION: Class I 9-mer I215L _191

SEQUENCE: 2

Glu Met Glu Asp Asp Thr Tyr Ile Leu

SEQ ID NO 3
LENGTH: 9
TYPE: Protein
OTHER INFORMATION: Class I 9-mer C475L_292

SEQUENCE: 3

Tyr Met Asp Tyr Ser Pro Pro Ile Phe

SEQ ID NO 4
LENGTH: 9
TYPE: Protein
OTHER INFORMATION: Class I 9-mer B475L_63

SEQUENCE: 4

Met Leu Asp Ser Phe Tyr Lys Tyr Phe

SEQ ID NO 5
LENGTH: 9
TYPE: Protein
OTHER INFORMATION: Class I 9-mer C257L_197

SEQUENCE: 5

Gln Met Glu Ile Lys Arg Phe Ile Lys

SEQ ID NO 6
LENGTH: 9
TYPE: Protein
OTHER INFORMATION: Class I 9-mer NP 1450L_334

SEQUENCE: 6

His Leu Asp Glu Val Gly Tyr Pro Ile

SEQ ID NO 7
LENGTH: 9
TYPE: Protein
OTHER INFORMATION: Class I 9-mer CP2475L_1032

SEQUENCE: 7

Met Met Asn Gln Thr Asn Tyr Ser Ile

SEQ ID NO 8
LENGTH: 9
TYPE: Protein
OTHER INFORMATION: Class I 9-mer B646L455

SEQUENCE: 8

Leu Met Ser Ala Leu Lys Trp Pro Ile

SEQ ID NO 9
LENGTH: 9
TYPE: Protein
OTHER INFORMATION: Class I 9-mer F778R 273

SEQUENCE: 9

Tyr Leu Glu Leu Trp His Gln Asp Ile

SEQ ID NO 10
LENGTH: 9
TYPE: Protein
OTHER INFORMATION: Class I 9-mer B407L_267

SEQUENCE: 10

Thr Ile Asp Ser Ser Met Gln Glu Ile

SEQ ID NO 11
LENGTH: 9 TYPE: Protein
OTHER INFORMATION: Class I 9-mer F334L_262

SEQUENCE: 11

Met Leu Phe Gln Tyr Ile Arg Tyr Phe

SEQ ID NO 12
LENGTH: 9
TYPE: Protein
OTHER INFORMATION: Class I 9-mer B646L579

SEQUENCE: 12

Ala Met Met Ile Thr Phe Ala Leu Lys

SEQ ID NO 13
LENGTH: 9
TYPE: Protein
OTHER INFORMATION: Class I 9-mer B318L_36

SEQUENCE: 13

Ala Leu Phe His Gly Ile His Pro Leu

SEQ ID NO 14
LENGTH: 9
TYPE: Protein
OTHER INFORMATION: Class I 9-mer MGF_505_11L _120

SEQUENCE: 14

Glu Leu Phe Glu Phe Phe His Leu Phe

SEQ ID NO 15
LENGTH: 9
TYPE: Protein
OTHER INFORMATION: Class I 9-mer NP868R 213

SEQUENCE: 15

Thr Leu Ile Ser Pro Asn His Leu Met

SEQ ID NO 16
LENGTH: 9
TYPE: Protein
OTHER INFORMATION: Class I 9-mer EP424R 264

SEQUENCE: 16

Asn Met Ile Leu Lys His Tyr Thr Leu

SEQ ID NO 17
LENGTH: 9
TYPE: Protein
OTHER INFORMATION: Class I 9-mer NP1450_325

SEQUENCE: 17

Ser Thr Ile Cys Gly Asn Ser Asp Leu

SEQ ID NO 18
LENGTH: 9
TYPE: Protein
OTHER INFORMATION: Class I 9-mer K145R 51

SEQUENCE: 18

Gln Leu Phe Leu Thr Met Tyr Lys Leu

SEQ ID NO 19
LENGTH: 9
TYPE: Protein
OTHER INFORMATION: Class I 9-mer MGF_300_1L_177

SEQUENCE: 19

Ser Leu Ser Thr Leu Tyr Cys Ile Phe

SEQ ID NO 20
LENGTH: 9
TYPE: Protein
OTHER INFORMATION: Class I 9-mer G1211R_852

SEQUENCE: 20

Gln Thr Asp Ala Trp Arg Pro Asp Lys

SEQ ID NO 21
LENGTH: 9
TYPE: Protein
OTHER INFORMATION: Class I 9-mer EP1242L_663

SEQUENCE: 21

Trp Leu Asp Val Gly Arg Leu Thr Arg

SEQ ID NO 22
LENGTH: 9
TYPE: Protein
OTHER INFORMATION: Class I 9-mer B438L_21

SEQUENCE: 22

Glu Ser Asp Leu Pro Arg His Asn Arg

SEQ ID NO 23
LENGTH: 9
TYPE: Protein
OTHER INFORMATION: Class I 9-mer I267L 220

SEQUENCE: 23

Met Val Asp Asn His Pro Phe Lys Lys

SEQ ID NO 24
LENGTH: 9
TYPE: Protein
OTHER INFORMATION: Class I 9-mer B962L_152

SEQUENCE: 24

Met Thr Asp Asp Glu Ile Ala Ser Arg

SEQ ID NO 25
LENGTH: 9
TYPE: Protein
OTHER INFORMATION: Class I 9-mer CP2475L2125

SEQUENCE: 25

Ser Leu Tyr Pro Thr Gln Phe Asp Tyr

SEQ ID NO 26
LENGTH: 9
TYPE: Protein
OTHER INFORMATION: Class I 9-mer M1249L_580

SEQUENCE: 26

Gln Leu Tyr Ala Val Ile Tyr Ile Tyr

SEQ ID NO 27
LENGTH: 9
TYPE: Protein
OTHER INFORMATION: Class I 9-mer DP79L _64

SEQUENCE: 27

Ser Ile Tyr Ile Met Val Val Glu Tyr

SEQ ID NO 28
LENGTH: 9
TYPE: Protein
OTHER INFORMATION: Class I 9-mer F778R 99

SEQUENCE: 28

Ala Ile Phe Asp Ser Tyr Ile Asp Tyr

SEQ ID NO 29
LENGTH: 9
TYPE: Protein
OTHER INFORMATION: Class I 9-mer P1192R_1030

SEQUENCE: 29

Glu Ile Leu Tyr Ala Trp Leu Pro Tyr

SEQ ID NO 30
LENGTH: 9
TYPE: Protein
OTHER INFORMATION: Class I 9-mer D339L_321

SEQUENCE: 30

Lys Arg His Glu Asn Ile Trp Met Leu

SEQ ID NO 31
LENGTH: 9
TYPE: Protein
OTHER INFORMATION: Class I 9-mer MGF_505_6R_305

SEQUENCE: 31

Ser Arg Lys Thr Leu Asn Leu Leu Leu

SEQ ID NO 32
LENGTH: 9
TYPE: Protein
OTHER INFORMATION: Class I 9-mer EP1242L_384

SEQUENCE: 32

Val Arg Lys Leu Arg Phe Leu Gly Leu

SEQ ID NO 33
LENGTH: 9
TYPE: Protein
OTHER INFORMATION: Class I 9-mer B438L _167

SEQUENCE: 33

Thr Arg Phe Ser Glu His Thr Lys Phe

SEQ ID NO 34
LENGTH: 9
TYPE: Protein
OTHER INFORMATION: Class I 9-mer B175L _144

SEQUENCE: 34

Met Arg Lys Glu Tyr Gly Gly Lys Leu

SEQ ID NO 35
LENGTH: 9
TYPE: Protein
OTHER INFORMATION: Class I 9-mer A859L_552

SEQUENCE: 35

Ser Glu Val Ala Tyr Pro Ile Asn Trp

SEQ ID NO 36
LENGTH: 9
TYPE: Protein
OTHER INFORMATION: Class I 9-mer I215L₋71

SEQUENCE: 36

Ser Glu Met Trp His Pro Asn Ile Tyr

SEQ ID NO 37
LENGTH: 9
TYPE: Protein
OTHER INFORMATION: Class I 9-mer S183L_ 25

SEQUENCE: 37

Ala Glu Leu Glu Ser Val Asn Tyr Tyr

SEQ ID NO 38
LENGTH: 9
TYPE: Protein
OTHER INFORMATION: Class I 9-mer B385R_44

SEQUENCE: 38

Gln Glu Tyr Phe Asn Phe Leu Met Ile

SEQ ID NO 39
LENGTH: 9
TYPE: Protein
OTHER INFORMATION: Class I 9-mer B125R_102

SEQUENCE: 39

Glu Glu Tyr Leu Arg Met His Phe Ile

SEQ ID NO 40
LENGTH: 17
TYPE: Protein
OTHER INFORMATION: Class II 9-mer Cluster B119L_49

SEQUENCE: 40

His His Ala Phe Ser Tyr Leu Thr Lys Asn Pro Leu Thr Leu Asn Asn Ser

SEQ ID NO 41
LENGTH: 19
TYPE: Protein
OTHER INFORMATION: Class II 9-mer Cluster B354L_250

SEQUENCE: 41

Lys Asn Ala Phe Val Ser Ile Phe Thr Asn Ala Ser Ile Cys Met Ser Asn Phe Ser

SEQ ID NO 42
LENGTH: 21
TYPE: Protein
OTHER INFORMATION: Class II 9-mer Cluster B646L_322

SEQUENCE: 42

Lys Leu Arg Phe Trp Phe Asn Glu Asn Val Asn Leu Ala Ile Pro Ser Val Ser Ile Pro Phe

SEQ ID NO 43
LENGTH: 17
TYPE: Protein
OTHER INFORMATION: Class II 9-mer Cluster B962L200

SEQUENCE: 43

Arg Ile Pro Phe Val Ile Leu Thr Ser Ala Thr Ile Asp Thr His Lys Tyr

SEQ ID NO 44
LENGTH: 18
TYPE: Protein
OTHER INFORMATION: Class II 9-mer Cluster B962L_68

SEQUENCE: 44

Val His Val Phe Arg Ile Leu Arg Asn Glu Asn Thr His Ser Phe Gln Lys Tyr

SEQ ID NO 45
LENGTH: 28
TYPE: Protein
OTHER INFORMATION: Class II 9-mer Cluster C717R 549

SEQUENCE: 45

Ala Ile Asp Phe Tyr Ala Ile Ala Arg Asn Leu Arg Ser Met Leu Ser Leu Asp Tyr Leu His Thr Ser Glu Val Lys Arg Asn

SEQ ID NO 46
LENGTH: 20
TYPE: Protein
OTHER INFORMATION: Class II 9-mer Cluster CP2475L_551

SEQUENCE: 46

Tyr Tyr Tyr Tyr Val Ala Gln Ile Tyr Ser Asn Leu Thr His Asn Lys Gln Glu Phe Gln

SEQ ID NO 47
LENGTH: 19
TYPE: Protein
OTHER INFORMATION: Class II 9-mer Cluster E248R 69

SEQUENCE: 47

Glu Glu Asn Phe Ile Thr Asn Leu Ser Asn Gln Ile Thr Gln Asn Leu Lys Asp Gln

SEQ ID NO 48
LENGTH: 22
TYPE: Protein
OTHER INFORMATION: Class II 9-mer Cluster E301R 44

SEQUENCE: 48

Pro Leu Ile Phe Lys Asn Leu Phe Ile Tyr Phe Lys Asn Leu Lys Ser Lys Asn Ile Leu Val Arg

SEQ ID NO 49
LENGTH: 18
TYPE: Protein
OTHER INFORMATION: Class II 9-mer Cluster E301R_253

SEQUENCE: 49

Asn Val Lys Ile Ala His Ile Lys Ser Leu Ala Ser Ala Met Val Thr Asp Lys

SEQ ID NO 50
LENGTH: 19
TYPE: Protein
OTHER INFORMATION: Class II 9-mer Cluster EP1242L_23

SEQUENCE: 50

Glu Ala Asp Met Leu Ser Phe Ile Ser Ala Ala Val Asn Ser Thr Gly Leu Ile Gly

SEQ ID NO 51
LENGTH: 23
TYPE: Protein
OTHER INFORMATION: Class II 9-mer Cluster EP1242L _459

SEQUENCE: 51

Gln Arg Asn Ile Ile Glu Ala Phe Ser Ala Ala Leu Ser Lys Asn Thr Ala Ser Asp Leu Asn Arg Ser

SEQ ID NO 52
LENGTH: 19
TYPE: Protein
OTHER INFORMATION: Class II 9-mer Cluster EP364R 111

SEQUENCE: 52

His Val Ala Tyr Ala Ser Ile Ile Thr Ala Met Thr His Leu Met Val Arg Asp His

SEQ ID NO 53
LENGTH: 17
TYPE: Protein
OTHER INFORMATION: Class II 9-mer Cluster EP424R 267

SEQUENCE: 53

Leu Lys His Tyr Thr Leu Asn His Ala Phe Thr Leu Ser Leu Ile Cys Val

SEQ ID NO 54
LENGTH: 18
TYPE: Protein
OTHER INFORMATION: Class II 9-mer Cluster F165R 76

SEQUENCE: 54

Asn Thr Asp Val Val Tyr Leu Ile Pro Ser Leu Thr Leu His Thr Pro Met Phe

SEQ ID NO 55
LENGTH: 17
TYPE: Protein
OTHER INFORMATION: Class II 9-mer Cluster F317L_237

SEQUENCE: 55

Leu Asp Ile Phe Met Met Leu Thr Ser Arg Arg Ser Leu Val Asn Pro Trp

SEQ ID NO 56
LENGTH: 18
TYPE: Protein
OTHER INFORMATION: Class II 9-mer Cluster G1211R_ 1023

SEQUENCE: 56

Ala Asp Leu Tyr Lys Glu Phe Phe Asn Asn Thr Thr Asn Pro Ile Glu Ser Phe

SEQ ID NO 57
LENGTH: 17
TYPE: Protein
OTHER INFORMATION: Class II 9-mer Cluster G1340L_134

SEQUENCE: 57

Arg Gln His Ile Phe Phe Phe Gln Asn Asn Ala Leu Gln Val Thr Tyr Arg

SEQ ID NO 58
LENGTH: 24
TYPE: Protein
OTHER INFORMATION: Class II 9-mer Cluster G1340L_1145

SEQUENCE: 58

Phe Leu Leu Ile Glu Phe Phe Asp Val Leu Tyr Gly Leu Gln Ser Asn Ser
Thr Arg Lys His Ile Glu Asn

SEQ ID NO 59
LENGTH: 19
TYPE: Protein
OTHER INFORMATION: Class II 9-mer Cluster K145R 29

SEQUENCE: 59

Glu Lys Glu Phe Ile Thr Leu Leu Asn Gln Ala Leu Ala Ser Thr Gln Leu
Tyr Arg

SEQ ID NO 60
LENGTH: 18
TYPE: Protein
OTHER INFORMATION: Class II 9-mer Cluster K145R 72

SEQUENCE: 60

Lys Gln Glu Tyr Leu Cys Leu Leu Ile Asn Pro Lys Leu Val Thr Lys Phe
Leu

SEQ ID NO 61
LENGTH: 17
TYPE: Protein
OTHER INFORMATION: Class II 9-mer Cluster NP1450L_337

SEQUENCE: 61

Glu Val Gly Tyr Pro Ile Ser Phe Ala Arg Thr Leu Gln Val Ala Glu Thr

SEQ ID NO 62
LENGTH: 24
TYPE: Protein
OTHER INFORMATION: Class II 9-mer Cluster NP1450L_1281

SEQUENCE: 62

Gln Gly Lys Leu Val Arg Leu Asp Asn Ile Tyr Ala Ile Lys Thr Asn Gly
Thr Asn Ile Phe Gly Ala Met

SEQ ID NO 63
LENGTH: 16
TYPE: Protein
OTHER INFORMATION: Class II 9-mer Cluster NP868R_592

SEQUENCE: 63

Lys Thr Gly Ile Tyr Arg Ala Gln Thr Ala Leu Ile Ser Phe Ile Lys

SEQ ID NO 64
LENGTH: 19
TYPE: Protein
OTHER INFORMATION: Class II 9-mer Cluster P1192R_ 1119

SEQUENCE: 64

Gln Gly Cys Tyr Thr Tyr Ile Leu Ser Leu Gln Ala Arg Glu Leu Leu Ile
Ala Ala

SEQ ID NO 65
LENGTH: 27
TYPE: Protein
OTHER INFORMATION: Class II 9-mer Cluster PEP402R 328BURMAKINA

SEQUENCE: 65

Tyr Ser Pro Pro Lys Pro Leu Pro Ser Ile Pro Leu Leu Pro Asn Ile Pro
Pro Leu Ser Thr Gln Asn Ile Ser Leu Ile

SEQ ID NO 66
LENGTH: 351
TYPE: Protein
OTHER INFORMATION: Class I ASFV Construct Before Vaccine CAD

SEQUENCE: 66

Met Leu Phe Gln Tyr Ile Arg Tyr Phe Glu Leu Phe Glu Phe Phe His Leu
Phe Met Leu Asp Ser Phe Tyr Lys Tyr Phe Trp Leu Asp Val Gly Arg Leu
Thr Arg Ala Leu Phe His Gly Ile His Pro Leu Ser Leu Tyr Pro Thr Gln
Phe Asp Tyr Met Val Asp Asn His Pro Phe Lys Lys Glu Leu Asp Ala Arg
Leu Trp Ile Met Met Met Asn Gln Thr Asn Tyr Ser Ile Ser Leu Ser Thr
Leu Tyr Cys Ile Phe Lys Arg His Glu Asn Ile Trp Met Leu Glu Met Glu
Asp Asp Thr Tyr Ile Leu Gln Leu Phe Leu Thr Met Tyr Lys Leu Tyr Met
Asp Tyr Ser Pro Pro Ile Phe Val Arg Lys Leu Arg Phe Leu Gly Leu Ser
Arg Lys Thr Leu Asn Leu Leu Leu Met Arg Lys Glu Tyr Gly Gly Lys Leu
Gln Leu Tyr Ala Val Ile Tyr Ile Tyr Thr Arg Phe Ser Glu His Thr Lys
Phe Ala Met Met Ile Thr Phe Ala Leu Lys Met Thr Asp Asp Glu Ile Ala
Ser Arg Asn Met Ile Leu Lys His Tyr Thr Leu Ser Ile Tyr Ile Met Val
Val Glu Tyr Thr Leu Ile Ser Pro Asn His Leu Met Gln Met Glu Ile Lys
Arg Phe Ile Lys Ser Thr Ile Cys Gly Asn Ser Asp Leu His Leu Asp Glu
Val Gly Tyr Pro Ile Gln Thr Asp Ala Trp Arg Pro Asp Lys Ala Ile Phe
Asp Ser Tyr Ile Asp Tyr Glu Ile Leu Tyr Ala Trp Leu Pro Tyr Leu Met
Ser Ala Leu Lys Trp Pro Ile Tyr Leu Glu Leu Trp His Gln Asp Ile Thr
Ile Asp Ser Ser Met Gln Glu Ile Glu Ser Asp Leu Pro Arg His Asn Arg
Ser Glu Met Trp His Pro Asn Ile Tyr Ala Glu Leu Glu Ser Val Asn Tyr
Tyr Ser Glu Val Ala Tyr Pro Ile Asn Trp Gln Glu Tyr Phe Asn Phe Leu
Met Ile Glu Glu Tyr Leu Arg Met His Phe Ile

SEQ ID NO 67
LENGTH: 399
TYPE: Protein
OTHER INFORMATION: Class I ASFV Construct After Vaccine CAD

SEQUENCE: 67

Met Leu Asp Ser Phe Tyr Lys Tyr Phe Ala Ala Tyr Gln Met Glu Ile Lys
Arg Phe Ile Lys Ala Ala Tyr Ser Ile Tyr Ile Met Val Val Glu Tyr Ala
Ala Tyr Gln Glu Tyr Phe Asn Phe Leu Met Ile Ala Ala Tyr Tyr Met Asp
Tyr Ser Pro Pro Ile Phe Gln Leu Phe Leu Thr Met Tyr Lys Leu Ala Ala
Tyr Met Leu Phe Gln Tyr Ile Arg Tyr Phe Glu Ser Asp Leu Pro Arg His
Asn Arg Thr Arg Phe Ser Glu His Thr Lys Phe Gln Thr Asp Ala Trp Arg
Pro Asp Lys Ala Leu Phe His Gly Ile His Pro Leu Glu Glu Tyr Leu Arg
Met His Phe Ile Ala Ala Tyr Ala Glu Leu Glu Ser Val Asn Tyr Tyr Thr
Ile Asp Ser Ser Met Gln Glu Ile Met Thr Asp Asp Glu Ile Ala Ser Arg
Ser Thr Ile Cys Gly Asn Ser Asp Leu Thr Leu Ile Ser Pro Asn His Leu
Met His Leu Asp Glu Val Gly Tyr Pro Ile Lys Arg His Glu Asn Ile Trp
Met Leu Ala Ala Tyr Ser Glu Met Trp His Pro Asn Ile Tyr Ser Glu Val
Ala Tyr Pro Ile Asn Trp Ser Arg Lys Thr Leu Asn Leu Leu Leu Ala Ala
Tyr Glu Met Glu Asp Asp Thr Tyr Ile Leu Glu Leu Asp Ala Arg Leu Trp
Ile Met Ala Ala Tyr Met Met Asn Gln Thr Asn Tyr Ser Ile Ala Ala Tyr
Ser Leu Ser Thr Leu Tyr Cys Ile Phe Ala Ala Tyr Met Val Asp Asn His
Pro Phe Lys Lys Ala Ile Phe Asp Ser Tyr Ile Asp Tyr Trp Leu Asp Val
Gly Arg Leu Thr Arg Ala Ala Tyr Glu Ile Leu Tyr Ala Trp Leu Pro Tyr
Ser Leu Tyr Pro Thr Gln Phe Asp Tyr Leu Met Ser Ala Leu Lys Trp Pro
Ile Val Arg Lys Leu Arg Phe Leu Gly Leu Met Arg Lys Glu Tyr Gly Gly
Lys Leu Ala Ala Tyr Ala Met Met Ile Thr Phe Ala Leu Lys Ala Ala Tyr
Asn Met Ile Leu Lys His Tyr Thr Leu Ala Ala Tyr Glu Leu Phe Glu Phe
Phe His Leu Phe Tyr Leu Glu Leu Trp His Gln Asp Ile Ala Ala Tyr Gln
Leu Tyr Ala Val Ile Tyr Ile Tyr

SEQ ID NO 68
LENGTH: 1197
TYPE: DNA
OTHER INFORMATION: Class I ASFV Construct After Vaccine CAD Back-Translation

SEQUENCE: 68

atgctggacagcttctacaagtacttcgccgcctaccagatggaaatcaa
gcggttcatcaaggccgcctacagcatctacatcatggtggtggaatacg
ccgcctatcaagagtacttcaacttcctcatgattgccgcctactacatg
gactacagccctccaatcttccagctgtttctgaccatgtacaagctggc
cgcttacatgctgttccagtacatccggtacttcgagagcgatctgcccc
ggcacaaccggacaagattcagcgagcacaccaagttccagaccgacgct
tggaggcccgacaaggctctgtttcacggcattcaccctctggaagagta
tctgcggatgcactttatcgccgcttacgccgagctggaaagcgtgaact
actacaccatcgacagcagcatgcaagagatcatgaccgacgacgagatc
gccagcagaagcaccatctgcggcaacagcgatctgacactgatcagccc
caaccatctgatgcatctggacgaagtgggctaccccatcaagcggcacg
agaacatttggatgctggccgcctattccgagatgtggcaccccaacatc
tacagcgaagtggcctatcctatcaactggtcccgcaagacactgaatct
gctgctggctgcctacgaaatggaagatgacacttacatcctcgagctgg
acgcccggctgtggatcatggcagcctacatgatgaaccagaccaactac
tctatcgctgcctactctctgagcacactgtactgcatctttgctgccta
catggtggacaatcaccccttcaagaaggccatcttcgacagctacatcg
actactggctggacgtgggcagactgaccagagccgcctatgagattctg
tacgcttggctgccctattctctgtaccccacacagttcgactatctgat
gagcgctctgaagtggcccatcgtgcggaagctgagatttctgggactga
tgcggaaagagtacggcggcaaactggccgcatacgccatgatgatcaca
ttcgcactgaaggctgcttacaacatgatcctcaagcactacaccctcgc
agcctacgagctgttcgagttcttccatctgttctatctggaactgtggc
accaagatattgccgcttaccagctgtacgccgtgatctacatctac

SEQ ID NO 69
LENGTH: 5067
TYPE: DNA
OTHER INFORMATION: Class I Plasmid

SEQUENCE: 69

gctagcaccgttggtttccgtagtgtagtggttatcacgttcgcctaaca
cgcgaaaggtccccggttcgaaaccgggcactacaaaccaacaacgttaa
aaaacaggtcctccccatactctttcattgtacacaccgcaagctcgaca
atcatcggattgaagcattgtcgcacacatcttccacacaggatcagtac
ctgctttcgcttttaaccaaggcttttctccaagggatatttatagtctc
aaaacacacaattactttacagttagggtgagtttccttttgtgctgttt
tttaaaataataatttagtatttgtatctcttatagaaatccaagcctat
catgtaaaatgtagctagtattaaaaagaacagattatctgtcttttatc
gcacattaagcctctatagttactaggaaatattatatgcaaattaaccg
gggcaggggagtagccgagcttctcccacaagtctgtgcgagggggccgg
cgcgggcctagagatggcggcgtcggatcggccagcccgcctaatgagcg
ggcttttttttcttagggtgcaaaaggagagcctgtaagcgggcactctt
ccgtggtctggtggataaattcgcaagggtatcatggcggacgaccgggg
ttcgagccccgtatccggccgtccgccgtgatccatgcggttaccgcccg
cgtgtcgaacccaggtgtgcgacgtcagacaacgggggagtgctcctttt
ggcttccttcccctaccggtctgcctcgcgcgtttcggtgatgacggtga
aaacctctgacacatgcagctcccggagacggtcacagcttgtctgtaag
cggatgccgggagcagacaagcccgtcagggcgcgtcagcgggtgttggc
gggtgtcggggcgcagccatgacccagtcacgtagcgatagcggagtgta
tactggcttaactatgcggcatcagagcagattgtactgagagtgcacca
tatgcggtgtgaaataccgcacagatgcgtaaggagaaaataccgcatca
ggcgctcttccgcttcctcgctcactgactcgctgcgctcggtcgttcgg
ctgcggcgagcggtatcagctcactcaaaggcggtaatacggttatccac
agaatcaggggataacgcaggaaagaacatgtgagcaaaaggccagcaaa
aggccaggaaccgtaaaaaggccgcgttgctggcgtttttccataggctc
cgcccccctgacgagcatcacaaaaatcgacgctcaagtcagaggtggcg
aaacccgacaggactataaagataccaggcgtttccccctggaagctccc
tcgtgcgctctcctgttccgaccctgccgcttaccggatacctgtccgcc
tttctcccttcgggaagcgtggcgctttctcatagctcacgctgtaggta
tctcagttcggtgtaggtcgttcgctccaagctgggctgtgtgcacgaac
cccccgttcagcccgaccgctgcgccttatccggtaactatcgtcttgag
tccaacccggtaagacacgacttatcgccactggcagcagccactggtaa
caggattagcagagcgaggtatgtaggcggtgctacagagttcttgaagt
ggtggcctaactacggctacactagaagaacagtatttggtatctgcgct
ctgctgaagccagttaccttcggaaaaagagttggtagctcttgatccgg
caaacaaaccaccgctggtagcggtggtttttttgtttgcaagcagcaga
ttacgcgcagaaaaaaaggatctcaagaagatcctttgatcttttctacg
gggtctgacgctcagtggaacgaaaactcacgttaagggattttggtcat
gagattatcaaaaaggatcttcacctagatccttttaaattaaaaatgaa
gttttaaatcaatctaaagtatatatgagtaaacttggtctgacagttac
caatgcttaatcagtgaggcacctatctcagcgatctgtctatttcgttc
atccatagttgcctgactcctgcaaaccacgttgtggtagaattggtaaa
gagagtcgtgtaaaatatcgagttcgcacatcttgttgtctgattattga
tttttggcgaaaccatttgatcatatgacaagatgtgtatctaccttaac
ttaatgattttgataaaaatcattaggtacccctgatcactgtggaatgt
gtgtcagttagggtgtggaaagtccccaggctccccagcaggcagaagta
tgcaaagcatgcatctcaattagtcagcaaccaggtgtggaaagtcccca
ggctccccagcaggcagaagtatgcaaagcatgcatctcaattagtcagc
aaccatagtcccgcccctaactccgcccatcccgcccctaactccgccca
gttacggggtcattagttcatagcccatatatggagttccgcgttacata
acttacggtaaatggcccgcctggctgaccgcccaacgacccccgcccat
tgacgtcaataatgacgtatgttcccatagtaacgccaatagggactttc
cattgacgtcaatgggtggagtatttacggtaaactgcccacttggcagt
acatcaagtgtatcatatgccaagtacgccccctattgacgtcaatgacg
gtaaatggcccgcctggcattatgcccagtacatgaccttatgggacttt
cctacttggcagtacatctacgtattagtcatcgctattaccatggtgat
gcggttttggcagtacatcaatgggcgtggatagcggtttgactcacggg
gatttccaagtctccaccccattgacgtcaatgggagtttgttttggcac
caaaatcaacgggactttccaaaatgtcgtaacaactccgccccattgac
gcaaatgggcggtaggcgtgtacggtgggaggtctatataagcagagctc
gtttagtgaaccgtcagatcgcctggagacgccatccacgctgttttgac
ctccatagaagacaccgggaccgatccagcctccgcggctcgcatctctc
cttcacgcgcccgccgccctacctgaggccgccatccacgccggttgagt
cgcgttctgccgcctcccgcctgtggtgcctcctgaactgcgtccgccgt
ctaggtaagtttaaagctcaggtcgagaccgggcctttgtccggcgctcc
cttggagcctacctagactcagccggctctccacgctttgcctgaccctg
cttgctcaactctagttctctcgttaacttaatgagacagatagaaactg
gtcttgtagaaacagagtagtcgcctgcttttctgccaggtgctgacttc
tctcccctgggcttttttctttttctcaggttgaaaagaagaagacgaag
aagacgaagaagacaaaccgtcgtcgagaagccaccatgcagattttcgt
caagaccctgacgggcaagaccatcactcttgaggtcgagcccagtgaca
ccatcgagaatgtcaaggccaagatccaagacaaggaaggcatcccacct
gaccagcagaggctgatattcgcgggcaaacagctggaggatggccgcac
cctgtccgactacaacatccagaaagagtccaccttgcacctggtgctgc
gtctgcgcggtgccgtcgacatgctggacagcttctacaagtacttcgcc
gcctaccagatggaaatcaagcggttcatcaaggccgcctacagcatcta
catcatggtggtggaatacgccgcctatcaagagtacttcaacttcctca
tgattgccgcctactacatggactacagccctccaatcttccagctgttt
ctgaccatgtacaagctggccgcttacatgctgttccagtacatccggta
cttcgagagcgatctgccccggcacaaccggacaagattcagcgagcaca
ccaagttccagaccgacgcttggaggcccgacaaggctctgtttcacggc
attcaccctctggaagagtatctgcggatgcactttatcgccgcttacgc
cgagctggaaagcgtgaactactacaccatcgacagcagcatgcaagaga
tcatgaccgacgacgagatcgccagcagaagcaccatctgcggcaacagc
gatctgacactgatcagccccaaccatctgatgcatctggacgaagtggg
ctaccccatcaagcggcacgagaacatttggatgctggccgcctattccg
agatgtggcaccccaacatctacagcgaagtggcctatcctatcaactgg
tcccgcaagacactgaatctgctgctggctgcctacgaaatggaagatga
cacttacatcctcgagctggacgcccggctgtggatcatggcagcctaca
tgatgaaccagaccaactactctatcgctgcctactctctgagcacactg
tactgcatctttgctgcctacatggtggacaatcaccccttcaagaaggc
catcttcgacagctacatcgactactggctggacgtgggcagactgacca
gagccgcctatgagattctgtacgcttggctgccctattctctgtacccc
acacagttcgactatctgatgagcgctctgaagtggcccatcgtgcggaa
gctgagatttctgggactgatgcggaaagagtacggcggcaaactggccg
catacgccatgatgatcacattcgcactgaaggctgcttacaacatgatc
ctcaagcactacaccctcgcagcctacgagctgttcgagttcttccatct
gttctatctggaactgtggcaccaagatattgccgcttaccagctgtacg
ccgtgatctacatctactaaagatctttttccctctgccaaaaattatgg
ggacatcatgaagccccttgagcatctgacttctggctaataaaggaaat
ttattttcattgcaatagtgtgttggaattttttgtgtctctcactcgga
aggacataagggcggcc

SEQ ID NO 70
LENGTH: 511
TYPE: Protein
OTHER INFORMATION: Class II ASFV Construct Before Vaccine CAD

SEQUENCE: 70

Ala Ile Asp Phe Tyr Ala Ile Ala Arg Asn Leu Arg Ser Met Leu Ser Leu
Asp Tyr Leu His Thr Ser Glu Val Lys Arg Asn Phe Leu Leu Ile Glu Phe
Phe Asp Val Leu Tyr Gly Leu Gln Ser Asn Ser Thr Arg Lys His Ile Glu
Asn Arg Gln His Ile Phe Phe Phe Gln Asn Asn Ala Leu Gln Val Thr Tyr
Arg Gln Arg Asn Ile Ile Glu Ala Phe Ser Ala Ala Leu Ser Lys Asn Thr
Ala Ser Asp Leu Asn Arg Ser Lys Leu Arg Phe Trp Phe Asn Glu Asn Val
Asn Leu Ala Ile Pro Ser Val Ser Ile Pro Phe Gln Gly Lys Leu Val Arg
Leu Asp Asn Ile Tyr Ala Ile Lys Thr Asn Gly Thr Asn Ile Phe Gly Ala
Met Glu Glu Asn Phe Ile Thr Asn Leu Ser Asn Gln Ile Thr Gln Asn Leu
Lys Asp Gln Tyr Tyr Tyr Tyr Val Ala Gln Ile Tyr Ser Asn Leu Thr His
Asn Lys Gln Glu Phe Gln His Val Ala Tyr Ala Ser Ile Ile Thr Ala Met
Thr His Leu Met Val Arg Asp His Leu Asp Ile Phe Met Met Leu Thr Ser
Arg Arg Ser Leu Val Asn Pro Trp Tyr Ser Pro Pro Lys Pro Leu Pro Ser
Ile Pro Leu Leu Pro Asn Ile Pro Pro Leu Ser Thr Gln Asn Ile Ser Leu
Ile Gln Gly Cys Tyr Thr Tyr Ile Leu Ser Leu Gln Ala Arg Glu Leu Leu
Ile Ala Ala Asn Thr Asp Val Val Tyr Leu Ile Pro Ser Leu Thr Leu His
Thr Pro Met Phe Glu Lys Glu Phe Ile Thr Leu Leu Asn Gln Ala Leu Ala
Ser Thr Gln Leu Tyr Arg Pro Leu Ile Phe Lys Asn Leu Phe Ile Tyr Phe
Lys Asn Leu Lys Ser Lys Asn Ile Leu Val Arg Val His Val Phe Arg Ile
Leu Arg Asn Glu Asn Thr His Ser Phe Gln Lys Tyr Glu Ala Asp Met Leu
Ser Phe Ile Ser Ala Ala Val Asn Ser Thr Gly Leu Ile Gly Lys Asn Ala
Phe Val Ser Ile Phe Thr Asn Ala Ser Ile Cys Met Ser Asn Phe Ser Glu
Val Gly Tyr Pro Ile Ser Phe Ala Arg Thr Leu Gln Val Ala Glu Thr Lys
Gln Glu Tyr Leu Cys Leu Leu Ile Asn Pro Lys Leu Val Thr Lys Phe Leu
Ala Asp Leu Tyr Lys Glu Phe Phe Asn Asn Thr Thr Asn Pro Ile Glu Ser
Phe Asn Val Lys Ile Ala His Ile Lys Ser Leu Ala Ser Ala Met Val Thr
Asp Lys Arg Ile Pro Phe Val Ile Leu Thr Ser Ala Thr Ile Asp Thr His
Lys Tyr His His Ala Phe Ser Tyr Leu Thr Lys Asn Pro Leu Thr Leu Asn
Asn Ser Lys Thr Gly Ile Tyr Arg Ala Gln Thr Ala Leu Ile Ser Phe Ile
Lys Leu Lys His Tyr Thr Leu Asn His Ala Phe Thr Leu Ser Leu Ile Cys
Val

SEQ ID NO 71
LENGTH: 511
TYPE: Protein
OTHER INFORMATION: Class II ASFV Construct After Vaccine CAD

SEQUENCE: 71

Gln Gly Lys Leu Val Arg Leu Asp Asn Ile Tyr Ala Ile Lys Thr Asn Gly
Thr Asn Ile Phe Gly Ala Met Ala Ile Asp Phe Tyr Ala Ile Ala Arg Asn
Leu Arg Ser Met Leu Ser Leu Asp Tyr Leu His Thr Ser Glu Val Lys Arg
Asn Phe Leu Leu Ile Glu Phe Phe Asp Val Leu Tyr Gly Leu Gln Ser Asn
Ser Thr Arg Lys His Ile Glu Asn Lys Asn Ala Phe Val Ser Ile Phe Thr
Asn Ala Ser Ile Cys Met Ser Asn Phe Ser Ala Asp Leu Tyr Lys Glu Phe
Phe Asn Asn Thr Thr Asn Pro Ile Glu Ser Phe Leu Asp Ile Phe Met Met
Leu Thr Ser Arg Arg Ser Leu Val Asn Pro Trp Lys Thr Gly Ile Tyr Arg
Ala Gln Thr Ala Leu Ile Ser Phe Ile Lys Gln Gly Cys Tyr Thr Tyr Ile
Leu Ser Leu Gln Ala Arg Glu Leu Leu Ile Ala Ala Tyr Tyr Tyr Tyr Val
Ala Gln Ile Tyr Ser Asn Leu Thr His Asn Lys Gln Glu Phe Gln Glu Glu
Asn Phe Ile Thr Asn Leu Ser Asn Gln Ile Thr Gln Asn Leu Lys Asp Gln
Pro Leu Ile Phe Lys Asn Leu Phe Ile Tyr Phe Lys Asn Leu Lys Ser Lys
Asn Ile Leu Val Arg His His Ala Phe Ser Tyr Leu Thr Lys Asn Pro Leu
Thr Leu Asn Asn Ser Tyr Ser Pro Pro Lys Pro Leu Pro Ser Ile Pro Leu
Leu Pro Asn Ile Pro Pro Leu Ser Thr Gln Asn Ile Ser Leu Ile Glu Val
Gly Tyr Pro Ile Ser Phe Ala Arg Thr Leu Gln Val Ala Glu Thr Glu Lys
Glu Phe Ile Thr Leu Leu Asn Gln Ala Leu Ala Ser Thr Gln Leu Tyr Arg
Val His Val Phe Arg Ile Leu Arg Asn Glu Asn Thr His Ser Phe Gln Lys
Tyr Arg Gln His Ile Phe Phe Phe Gln Asn Asn Ala Leu Gln Val Thr Tyr
Arg Lys Leu Arg Phe Trp Phe Asn Glu Asn Val Asn Leu Ala Ile Pro Ser
Val Ser Ile Pro Phe Arg Ile Pro Phe Val Ile Leu Thr Ser Ala Thr Ile
Asp Thr His Lys Tyr Asn Val Lys Ile Ala His Ile Lys Ser Leu Ala Ser
Ala Met Val Thr Asp Lys Glu Ala Asp Met Leu Ser Phe Ile Ser Ala Ala
Val Asn Ser Thr Gly Leu Ile Gly His Val Ala Tyr Ala Ser Ile Ile Thr
Ala Met Thr His Leu Met Val Arg Asp His Leu Lys His Tyr Thr Leu Asn
His Ala Phe Thr Leu Ser Leu Ile Cys Val Lys Gln Glu Tyr Leu Cys Leu
Leu Ile Asn Pro Lys Leu Val Thr Lys Phe Leu Gln Arg Asn Ile Ile Glu
Ala Phe Ser Ala Ala Leu Ser Lys Asn Thr Ala Ser Asp Leu Asn Arg Ser
Asn Thr Asp Val Val Tyr Leu Ile Pro Ser Leu Thr Leu His Thr Pro Met
Phe

SEQ ID NO 72
LENGTH: 1533
TYPE: DNA
OTHER INFORMATION: Class II ASFV Construct After Vaccine CAD Back-Translation

SEQUENCE: 72

caaggcaagcttgtgcggctggacaacatctacgccatcaagaccaacgg
caccaacatcttcggcgccatggccatcgacttctacgctatcgccagaa
atctgcggagcatgctgtctctggactatctgcacaccagcgaagtgaag
cggaactttctgctgatcgagttcttcgacgtgctgtacggactgcagag
caacagcacccggaagcacatcgagaacaagaacgccttcgtgtccatct
tcaccaacgccagcatctgcatgagcaacttcagcgccgatctgtacaaa
gagttcttcaacaacaccaccaatccgatcgagagcttcctcgacatctt
catgatgctgaccagccggcggagcctcgtgaacccttggaaaaccggaa
tctacagagcccagaccgctctgatcagcttcatcaagcaaggctgctac
acttacatcctcagcctccaagccagagagctgctgattgccgcctacta
ctactatgtggcccaaatctacagcaatctgacccacaacaagcaagagt
tccaagaggaaaacttcatcaccaatctgagcaaccagatcacccagaat
ctgaaggaccagcctctgatcttcaagaatctgttcatctactttaagaa
tctcaagagcaagaacatcctcgtgcggcaccacgccttcagctatctga
ccaagaatcctctgacactgaacaacagctacagccctcctaagcctctg
cctagcatccctctgctgccaaacatccctccactgagcacccagaacat
cagtctgatcgaagtgggctaccccatcagcttcgcccggacactgcaag
tggccgagacagagaaagagttcatcacgctgctgaatcaagctctggcc
agcacacagctgtaccgggtgcacgtgttccggattctgcggaacgagaa
cacccacagcttccagaagtaccggcagcacatcttcttcttccaaaaca
acgccctccaagtgacataccggaagctgcggttctggttcaacgagaat
gtgaatctggctatcccctccgtgtctatcccctttcggatccccttcgt
gattctgacaagcgccaccatcgacacccacaagtacaacgtgaagatcg
cccacatcaagagtctggcctccgccatggtcaccgacaaagaggccgat
atgctgagcttcatctctgccgccgtgaacagcaccggactgattggaca
tgtggcctacgcctccatcatcaccgccatgacacatctgatggtccgag
atcatctgaagcactacacactgaaccatgccttcacactgtctctgatc
tgcgtgaagcaagaatatctgtgcctcctcatcaaccccaagctggtcac
caagtttctgcagcggaacatcatcgaggccttctccgccgctctgtcca
agaatactgccagcgatctgaaccggtccaacaccgacgtggtgtatctg
atcccctctctgactctgcacacacccatgttc

SEQ ID NO 73
LENGTH: 5246
TYPE: DNA
OTHER INFORMATION: Class II Plasmid

SEQUENCE: 73

gctagcaccgttggtttccgtagtgtagtggttatcacgttcgcctaaca
cgcgaaaggtccccggttcgaaaccgggcactacaaaccaacaacgttaa
aaaacaggtcctccccatactctttcattgtacacaccgcaagctcgaca
atcatcggattgaagcattgtcgcacacatcttccacacaggatcagtac
ctgctttcgcttttaaccaaggcttttctccaagggatatttatagtctc
aaaacacacaattactttacagttagggtgagtttccttttgtgctgttt
tttaaaataataatttagtatttgtatctcttatagaaatccaagcctat
catgtaaaatgtagctagtattaaaaagaacagattatctgtcttttatc
gcacattaagcctctatagttactaggaaatattatatgcaaattaaccg
gggcaggggagtagccgagcttctcccacaagtctgtgcgagggggccgg
cgcgggcctagagatggcggcgtcggatcggccagcccgcctaatgagcg
ggcttttttttcttagggtgcaaaaggagagcctgtaagcgggcactctt
ccgtggtctggtggataaattcgcaagggtatcatggcggacgaccgggg
ttcgagccccgtatccggccgtccgccgtgatccatgcggttaccgcccg
cgtgtcgaacccaggtgtgcgacgtcagacaacgggggagtgctcctttt
ggcttccttcccctaccggtctgcctcgcgcgtttcggtgatgacggtga
aaacctctgacacatgcagctcccggagacggtcacagcttgtctgtaag
cggatgccgggagcagacaagcccgtcagggcgcgtcagcgggtgttggc
gggtgtcggggcgcagccatgacccagtcacgtagcgatagcggagtgta
tactggcttaactatgcggcatcagagcagattgtactgagagtgcacca
tatgcggtgtgaaataccgcacagatgcgtaaggagaaaataccgcatca
ggcgctcttccgcttcctcgctcactgactcgctgcgctcggtcgttcgg
ctgcggcgagcggtatcagctcactcaaaggcggtaatacggttatccac
agaatcaggggataacgcaggaaagaacatgtgagcaaaaggccagcaaa
aggccaggaaccgtaaaaaggccgcgttgctggcgtttttccataggctc
cgcccccctgacgagcatcacaaaaatcgacgctcaagtcagaggtggcg
aaacccgacaggactataaagataccaggcgtttccccctggaagctccc
tcgtgcgctctcctgttccgaccctgccgcttaccggatacctgtccgcc
tttctcccttcgggaagcgtggcgctttctcatagctcacgctgtaggta
tctcagttcggtgtaggtcgttcgctccaagctgggctgtgtgcacgaac
cccccgttcagcccgaccgctgcgccttatccggtaactatcgtcttgag
tccaacccggtaagacacgacttatcgccactggcagcagccactggtaa
caggattagcagagcgaggtatgtaggcggtgctacagagttcttgaagt
ggtggcctaactacggctacactagaagaacagtatttggtatctgcgct
ctgctgaagccagttaccttcggaaaaagagttggtagctcttgatccgg
caaacaaaccaccgctggtagcggtggtttttttgtttgcaagcagcaga
ttacgcgcagaaaaaaaggatctcaagaagatcctttgatcttttctacg
gggtctgacgctcagtggaacgaaaactcacgttaagggattttggtcat
gagattatcaaaaaggatcttcacctagatccttttaaattaaaaatgaa
gttttaaatcaatctaaagtatatatgagtaaacttggtctgacagttac
caatgcttaatcagtgaggcacctatctcagcgatctgtctatttcgttc
atccatagttgcctgactcctgcaaaccacgttgtggtagaattggtaaa
gagagtcgtgtaaaatatcgagttcgcacatcttgttgtctgattattga
tttttggcgaaaccatttgatcatatgacaagatgtgtatctaccttaac
ttaatgattttgataaaaatcattaggtacccctgatcactgtggaatgt
gtgtcagttagggtgtggaaagtccccaggctccccagcaggcagaagta
tgcaaagcatgcatctcaattagtcagcaaccaggtgtggaaagtcccca
ggctccccagcaggcagaagtatgcaaagcatgcatctcaattagtcagc
aaccatagtcccgcccctaactccgcccatcccgcccctaactccgccca
gttacggggtcattagttcatagcccatatatggagttccgcgttacata
acttacggtaaatggcccgcctggctgaccgcccaacgacccccgcccat
tgacgtcaataatgacgtatgttcccatagtaacgccaatagggactttc
cattgacgtcaatgggtggagtatttacggtaaactgcccacttggcagt
acatcaagtgtatcatatgccaagtacgccccctattgacgtcaatgacg
gtaaatggcccgcctggcattatgcccagtacatgaccttatgggacttt
cctacttggcagtacatctacgtattagtcatcgctattaccatggtgat
gcggttttggcagtacatcaatgggcgtggatagcggtttgactcacggg
gatttccaagtctccaccccattgacgtcaatgggagtttgttttggcac
caaaatcaacgggactttccaaaatgtcgtaacaactccgccccattgac
gcaaatgggcggtaggcgtgtacggtgggaggtctatataagcagagctc
gtttagtgaaccgtcagatcgcctggagacgccatccacgctgttttgac
ctccatagaagacaccgggaccgatccagcctccgcggctcgcatctctc
cttcacgcgcccgccgccctacctgaggccgccatccacgccggttgagt
cgcgttctgccgcctcccgcctgtggtgcctcctgaactgcgtccgccgt
ctaggtaagtttaaagctcaggtcgagaccgggcctttgtccggcgctcc
cttggagcctacctagactcagccggctctccacgctttgcctgaccctg
cttgctcaactctagttctctcgttaacttaatgagacagatagaaactg
gtcttgtagaaacagagtagtcgcctgcttttctgccaggtgctgacttc
tctcccctgggcttttttctttttctcaggttgaaaagaagaagacgaag
aagacgaagaagacaaagccgccaccatggatgcaatgaagagagggctc
tgctgtgtgctgctgctgtgtggagcagtcttcgtttcgcccagcggtac
cggatccgtcgaccaaggcaagcttgtgcggctggacaacatctacgcca
tcaagaccaacggcaccaacatcttcggcgccatggccatcgacttctac
gctatcgccagaaatctgcggagcatgctgtctctggactatctgcacac
cagcgaagtgaagcggaactttctgctgatcgagttcttcgacgtgctgt
acggactgcagagcaacagcacccggaagcacatcgagaacaagaacgcc
ttcgtgtccatcttcaccaacgccagcatctgcatgagcaacttcagcgc
cgatctgtacaaagagttcttcaacaacaccaccaatccgatcgagagct
tcctcgacatcttcatgatgctgaccagccggcggagcctcgtgaaccct
tggaaaaccggaatctacagagcccagaccgctctgatcagcttcatcaa
gcaaggctgctacacttacatcctcagcctccaagccagagagctgctga
ttgccgcctactactactatgtggcccaaatctacagcaatctgacccac
aacaagcaagagttccaagaggaaaacttcatcaccaatctgagcaacca
gatcacccagaatctgaaggaccagcctctgatcttcaagaatctgttca
tctactttaagaatctcaagagcaagaacatcctcgtgcggcaccacgcc
ttcagctatctgaccaagaatcctctgacactgaacaacagctacagccc
tcctaagcctctgcctagcatccctctgctgccaaacatccctccactga
gcacccagaacatcagtctgatcgaagtgggctaccccatcagcttcgcc
cggacactgcaagtggccgagacagagaaagagttcatcacgctgctgaa
tcaagctctggccagcacacagctgtaccgggtgcacgtgttccggattc
tgcggaacgagaacacccacagcttccagaagtaccggcagcacatcttc
ttcttccaaaacaacgccctccaagtgacataccggaagctgcggttctg
gttcaacgagaatgtgaatctggctatcccctccgtgtctatcccctttc
ggatccccttcgtgattctgacaagcgccaccatcgacacccacaagtac
aacgtgaagatcgcccacatcaagagtctggcctccgccatggtcaccga
caaagaggccgatatgctgagcttcatctctgccgccgtgaacagcaccg
gactgattggacatgtggcctacgcctccatcatcaccgccatgacacat
ctgatggtccgagatcatctgaagcactacacactgaaccatgccttcac
actgtctctgatctgcgtgaagcaagaatatctgtgcctcctcatcaacc
ccaagctggtcaccaagtttctgcagcggaacatcatcgaggccttctcc
gccgctctgtccaagaatactgccagcgatctgaaccggtccaacaccga
cgtggtgtatctgatcccctctctgactctgcacacacccatgttctaaa
gatctttttccctctgccaaaaattatggggacatcatgaagccccttga
gcatctgacttctggctaataaaggaaatttattttcattgcaatagtgt
gttggaattttttgtgtctctcactcggaaggacataagggcggcc

SEQ ID NO 74
LENGTH: 1197
TYPE: RNA
OTHER INFORMATION: Class I ASFV Construct After Vaccine CAD Back-Translation

SEQUENCE: 74

augcuggacagcuucuacaaguacuucgccgccuaccagauggaaaucaa
gcgguucaucaaggccgccuacagcaucuacaucauggugguggaauacg
ccgccuaucaagaguacuucaacuuccucaugauugccgccuacuacaug
gacuacagcccuccaaucuuccagcuguuucugaccauguacaagcuggc
cgcuuacaugcuguuccaguacauccgguacuucgagagcgaucugcccc
ggcacaaccggacaagauucagcgagcacaccaaguuccagaccgacgcu
uggaggcccgacaaggcucuguuucacggcauucacccucuggaagagua
ucugcggaugcacuuuaucgccgcuuacgccgagcuggaaagcgugaacu
acuacaccaucgacagcagcaugcaagagaucaugaccgacgacgagauc
gccagcagaagcaccaucugcggcaacagcgaucugacacugaucagccc
caaccaucugaugcaucuggacgaagugggcuaccccaucaagcggcacg
agaacauuuggaugcuggccgccuauuccgagauguggcaccccaacauc
uacagcgaaguggccuauccuaucaacuggucccgcaagacacugaaucu
gcugcuggcugccuacgaaauggaagaugacacuuacauccucgagcugg
acgcccggcuguggaucauggcagccuacaugaugaaccagaccaacuac
ucuaucgcugccuacucucugagcacacuguacugcaucuuugcugccua
caugguggacaaucaccccuucaagaaggccaucuucgacagcuacaucg
acuacuggcuggacgugggcagacugaccagagccgccuaugagauucug
uacgcuuggcugcccuauucucuguaccccacacaguucgacuaucugau
gagcgcucugaaguggcccaucgugcggaagcugagauuucugggacuga
ugcggaaagaguacggcggcaaacuggccgcauacgccaugaugaucaca
uucgcacugaaggcugcuuacaacaugauccucaagcacuacacccucgc
agccuacgagcuguucgaguucuuccaucuguucuaucuggaacuguggc
accaagauauugccgcuuaccagcuguacgccgugaucuacaucuac

SEQ ID NO 75
LENGTH: 5067
TYPE: RNA
OTHER INFORMATION: Class I Plasmid

SEQUENCE: 75

gcuagcaccguugguuuccguaguguagugguuaucacguucgccuaaca
cgcgaaagguccccgguucgaaaccgggcacuacaaaccaacaacguuaa
aaaacagguccuccccauacucuuucauuguacacaccgcaagcucgaca
aucaucggauugaagcauugucgcacacaucuuccacacaggaucaguac
cugcuuucgcuuuuaaccaaggcuuuucuccaagggauauuuauagucuc
aaaacacacaauuacuuuacaguuagggugaguuuccuuuugugcuguuu
uuuaaaauaauaauuuaguauuuguaucucuuauagaaauccaagccuau
cauguaaaauguagcuaguauuaaaaagaacagauuaucugucuuuuauc
gcacauuaagccucuauaguuacuaggaaauauuauaugcaaauuaaccg
gggcaggggaguagccgagcuucucccacaagucugugcgagggggccgg
cgcgggccuagagauggcggcgucggaucggccagcccgccuaaugagcg
ggcuuuuuuuucuuagggugcaaaaggagagccuguaagcgggcacucuu
ccguggucugguggauaaauucgcaaggguaucauggcggacgaccgggg
uucgagccccguauccggccguccgccgugauccaugcgguuaccgcccg
cgugucgaacccaggugugcgacgucagacaacgggggagugcuccuuuu
ggcuuccuuccccuaccggucugccucgcgcguuucggugaugacgguga
aaaccucugacacaugcagcucccggagacggucacagcuugucuguaag
cggaugccgggagcagacaagcccgucagggcgcgucagcggguguuggc
gggugucggggcgcagccaugacccagucacguagcgauagcggagugua
uacuggcuuaacuaugcggcaucagagcagauuguacugagagugcacca
uaugcggugugaaauaccgcacagaugcguaaggagaaaauaccgcauca
ggcgcucuuccgcuuccucgcucacugacucgcugcgcucggucguucgg
cugcggcgagcgguaucagcucacucaaaggcgguaauacgguuauccac
agaaucaggggauaacgcaggaaagaacaugugagcaaaaggccagcaaa
aggccaggaaccguaaaaaggccgcguugcuggcguuuuuccauaggcuc
cgccccccugacgagcaucacaaaaaucgacgcucaagucagagguggcg
aaacccgacaggacuauaaagauaccaggcguuucccccuggaagcuccc
ucgugcgcucuccuguuccgacccugccgcuuaccggauaccuguccgcc
uuucucccuucgggaagcguggcgcuuucucauagcucacgcuguaggua
ucucaguucgguguaggucguucgcuccaagcugggcugugugcacgaac
cccccguucagcccgaccgcugcgccuuauccgguaacuaucgucuugag
uccaacccgguaagacacgacuuaucgccacuggcagcagccacugguaa
caggauuagcagagcgagguauguaggcggugcuacagaguucuugaagu
gguggccuaacuacggcuacacuagaagaacaguauuugguaucugcgcu
cugcugaagccaguuaccuucggaaaaagaguugguagcucuugauccgg
caaacaaaccaccgcugguagcggugguuuuuuuguuugcaagcagcaga
uuacgcgcagaaaaaaaggaucucaagaagauccuuugaucuuuucuacg
gggucugacgcucaguggaacgaaaacucacguuaagggauuuuggucau
gagauuaucaaaaaggaucuucaccuagauccuuuuaaauuaaaaaugaa
guuuuaaaucaaucuaaaguauauaugaguaaacuuggucugacaguuac
caaugcuuaaucagugaggcaccuaucucagcgaucugucuauuucguuc
auccauaguugccugacuccugcaaaccacguugugguagaauugguaaa
gagagucguguaaaauaucgaguucgcacaucuuguugucugauuauuga
uuuuuggcgaaaccauuugaucauaugacaagauguguaucuaccuuaac
uuaaugauuuugauaaaaaucauuagguaccccugaucacuguggaaugu
gugucaguuaggguguggaaaguccccaggcuccccagcaggcagaagua
ugcaaagcaugcaucucaauuagucagcaaccagguguggaaagucccca
ggcuccccagcaggcagaaguaugcaaagcaugcaucucaauuagucagc
aaccauagucccgccccuaacuccgcccaucccgccccuaacuccgccca
guuacggggucauuaguucauagcccauauauggaguuccgcguuacaua
acuuacgguaaauggcccgccuggcugaccgcccaacgacccccgcccau
ugacgucaauaaugacguauguucccauaguaacgccaauagggacuuuc
cauugacgucaauggguggaguauuuacgguaaacugcccacuuggcagu
acaucaaguguaucauaugccaaguacgcccccuauugacgucaaugacg
guaaauggcccgccuggcauuaugcccaguacaugaccuuaugggacuuu
ccuacuuggcaguacaucuacguauuagucaucgcuauuaccauggugau
gcgguuuuggcaguacaucaaugggcguggauagcgguuugacucacggg
gauuuccaagucuccaccccauugacgucaaugggaguuuguuuuggcac
caaaaucaacgggacuuuccaaaaugucguaacaacuccgccccauugac
gcaaaugggcgguaggcguguacggugggaggucuauauaagcagagcuc
guuuagugaaccgucagaucgccuggagacgccauccacgcuguuuugac
cuccauagaagacaccgggaccgauccagccuccgcggcucgcaucucuc
cuucacgcgcccgccgcccuaccugaggccgccauccacgccgguugagu
cgcguucugccgccucccgccuguggugccuccugaacugcguccgccgu
cuagguaaguuuaaagcucaggucgagaccgggccuuuguccggcgcucc
cuuggagccuaccuagacucagccggcucuccacgcuuugccugacccug
cuugcucaacucuaguucucucguuaacuuaaugagacagauagaaacug
gucuuguagaaacagaguagucgccugcuuuucugccaggugcugacuuc
ucuccccugggcuuuuuucuuuuucucagguugaaaagaagaagacgaag
aagacgaagaagacaaaccgucgucgagaagccaccaugcagauuuucgu
caagacccugacgggcaagaccaucacucuugaggucgagcccagugaca
ccaucgagaaugucaaggccaagauccaagacaaggaaggcaucccaccu
gaccagcagaggcugauauucgcgggcaaacagcuggaggauggccgcac
ccuguccgacuacaacauccagaaagaguccaccuugcaccuggugcugc
gucugcgcggugccgucgacaugcuggacagcuucuacaaguacuucgcc
gccuaccagauggaaaucaagcgguucaucaaggccgccuacagcaucua
caucauggugguggaauacgccgccuaucaagaguacuucaacuuccuca
ugauugccgccuacuacauggacuacagcccuccaaucuuccagcuguuu
cugaccauguacaagcuggccgcuuacaugcuguuccaguacauccggua
cuucgagagcgaucugccccggcacaaccggacaagauucagcgagcaca
ccaaguuccagaccgacgcuuggaggcccgacaaggcucuguuucacggc
auucacccucuggaagaguaucugcggaugcacuuuaucgccgcuuacgc
cgagcuggaaagcgugaacuacuacaccaucgacagcagcaugcaagaga
ucaugaccgacgacgagaucgccagcagaagcaccaucugcggcaacagc
gaucugacacugaucagccccaaccaucugaugcaucuggacgaaguggg
cuaccccaucaagcggcacgagaacauuuggaugcuggccgccuauuccg
agauguggcaccccaacaucuacagcgaaguggccuauccuaucaacugg
ucccgcaagacacugaaucugcugcuggcugccuacgaaauggaagauga
cacuuacauccucgagcuggacgcccggcuguggaucauggcagccuaca
ugaugaaccagaccaacuacucuaucgcugccuacucucugagcacacug
uacugcaucuuugcugccuacaugguggacaaucaccccuucaagaaggc
caucuucgacagcuacaucgacuacuggcuggacgugggcagacugacca
gagccgccuaugagauucuguacgcuuggcugcccuauucucuguacccc
acacaguucgacuaucugaugagcgcucugaaguggcccaucgugcggaa
gcugagauuucugggacugaugcggaaagaguacggcggcaaacuggccg
cauacgccaugaugaucacauucgcacugaaggcugcuuacaacaugauc
cucaagcacuacacccucgcagccuacgagcuguucgaguucuuccaucu
guucuaucuggaacuguggcaccaagauauugccgcuuaccagcuguacg
ccgugaucuacaucuacuaaagaucuuuuucccucugccaaaaauuaugg
ggacaucaugaagccccuugagcaucugacuucuggcuaauaaaggaaau
uuauuuucauugcaauaguguguuggaauuuuuugugucucucacucgga
aggacauaagggcggcc

SEQ ID NO 76
LENGTH: 1533
TYPE: RNA
OTHER INFORMATION: Class II ASFV Construct After Vaccine CAD Back-Translation

SEQUENCE: 76

caaggcaagcuugugcggcuggacaacaucuacgccaucaagaccaacgg
caccaacaucuucggcgccauggccaucgacuucuacgcuaucgccagaa
aucugcggagcaugcugucucuggacuaucugcacaccagcgaagugaag
cggaacuuucugcugaucgaguucuucgacgugcuguacggacugcagag
caacagcacccggaagcacaucgagaacaagaacgccuucguguccaucu
ucaccaacgccagcaucugcaugagcaacuucagcgccgaucuguacaaa
gaguucuucaacaacaccaccaauccgaucgagagcuuccucgacaucuu
caugaugcugaccagccggcggagccucgugaacccuuggaaaaccggaa
ucuacagagcccagaccgcucugaucagcuucaucaagcaaggcugcuac
acuuacauccucagccuccaagccagagagcugcugauugccgccuacua
cuacuauguggcccaaaucuacagcaaucugacccacaacaagcaagagu
uccaagaggaaaacuucaucaccaaucugagcaaccagaucacccagaau
cugaaggaccagccucugaucuucaagaaucuguucaucuacuuuaagaa
ucucaagagcaagaacauccucgugcggcaccacgccuucagcuaucuga
ccaagaauccucugacacugaacaacagcuacagcccuccuaagccucug
ccuagcaucccucugcugccaaacaucccuccacugagcacccagaacau
cagucugaucgaagugggcuaccccaucagcuucgcccggacacugcaag
uggccgagacagagaaagaguucaucacgcugcugaaucaagcucuggcc
agcacacagcuguaccgggugcacguguuccggauucugcggaacgagaa
cacccacagcuuccagaaguaccggcagcacaucuucuucuuccaaaaca
acgcccuccaagugacauaccggaagcugcgguucugguucaacgagaau
gugaaucuggcuauccccuccgugucuauccccuuucggauccccuucgu
gauucugacaagcgccaccaucgacacccacaaguacaacgugaagaucg
cccacaucaagagucuggccuccgccauggucaccgacaaagaggccgau
augcugagcuucaucucugccgccgugaacagcaccggacugauuggaca
uguggccuacgccuccaucaucaccgccaugacacaucugaugguccgag
aucaucugaagcacuacacacugaaccaugccuucacacugucucugauc
ugcgugaagcaagaauaucugugccuccucaucaaccccaagcuggucac
caaguuucugcagcggaacaucaucgaggccuucuccgccgcucugucca
agaauacugccagcgaucugaaccgguccaacaccgacgugguguaucug
auccccucucugacucugcacacacccauguuc

SEQ ID NO 77
LENGTH: 5246
TYPE: RNA
OTHER INFORMATION: Class II Plasmid

SEQUENCE: 77

gcuagcaccguugguuuccguaguguagugguuaucacguucgccuaaca
cgcgaaagguccccgguucgaaaccgggcacuacaaaccaacaacguuaa
aaaacagguccuccccauacucuuucauuguacacaccgcaagcucgaca
aucaucggauugaagcauugucgcacacaucuuccacacaggaucaguac
cugcuuucgcuuuuaaccaaggcuuuucuccaagggauauuuauagucuc
aaaacacacaauuacuuuacaguuagggugaguuuccuuuugugcuguuu
uuuaaaauaauaauuuaguauuuguaucucuuauagaaauccaagccuau
cauguaaaauguagcuaguauuaaaaagaacagauuaucugucuuuuauc
gcacauuaagccucuauaguuacuaggaaauauuauaugcaaauuaaccg
gggcaggggaguagccgagcuucucccacaagucugugcgagggggccgg
cgcgggccuagagauggcggcgucggaucggccagcccgccuaaugagcg
ggcuuuuuuuucuuagggugcaaaaggagagccuguaagcgggcacucuu
ccguggucugguggauaaauucgcaaggguaucauggcggacgaccgggg
uucgagccccguauccggccguccgccgugauccaugcgguuaccgcccg
cgugucgaacccaggugugcgacgucagacaacgggggagugcuccuuuu
ggcuuccuuccccuaccggucugccucgcgcguuucggugaugacgguga
aaaccucugacacaugcagcucccggagacggucacagcuugucuguaag
cggaugccgggagcagacaagcccgucagggcgcgucagcggguguuggc
gggugucggggcgcagccaugacccagucacguagcgauagcggagugua
uacuggcuuaacuaugcggcaucagagcagauuguacugagagugcacca
uaugcggugugaaauaccgcacagaugcguaaggagaaaauaccgcauca
ggcgcucuuccgcuuccucgcucacugacucgcugcgcucggucguucgg
cugcggcgagcgguaucagcucacucaaaggcgguaauacgguuauccac
agaaucaggggauaacgcaggaaagaacaugugagcaaaaggccagcaaa
aggccaggaaccguaaaaaggccgcguugcuggcguuuuuccauaggcuc
cgccccccugacgagcaucacaaaaaucgacgcucaagucagagguggcg
aaacccgacaggacuauaaagauaccaggcguuucccccuggaagcuccc
ucgugcgcucuccuguuccgacccugccgcuuaccggauaccuguccgcc
uuucucccuucgggaagcguggcgcuuucucauagcucacgcuguaggua
ucucaguucgguguaggucguucgcuccaagcugggcugugugcacgaac
cccccguucagcccgaccgcugcgccuuauccgguaacuaucgucuugag
uccaacccgguaagacacgacuuaucgccacuggcagcagccacugguaa
caggauuagcagagcgagguauguaggcggugcuacagaguucuugaagu
gguggccuaacuacggcuacacuagaagaacaguauuugguaucugcgcu
cugcugaagccaguuaccuucggaaaaagaguugguagcucuugauccgg
caaacaaaccaccgcugguagcggugguuuuuuuguuugcaagcagcaga
uuacgcgcagaaaaaaaggaucucaagaagauccuuugaucuuuucuacg
gggucugacgcucaguggaacgaaaacucacguuaagggauuuuggucau
gagauuaucaaaaaggaucuucaccuagauccuuuuaaauuaaaaaugaa
guuuuaaaucaaucuaaaguauauaugaguaaacuuggucugacaguuac
caaugcuuaaucagugaggcaccuaucucagcgaucugucuauuucguuc
auccauaguugccugacuccugcaaaccacguugugguagaauugguaaa
gagagucguguaaaauaucgaguucgcacaucuuguugucugauuauuga
uuuuuggcgaaaccauuugaucauaugacaagauguguaucuaccuuaac
uuaaugauuuugauaaaaaucauuagguaccccugaucacuguggaaugu
gugucaguuaggguguggaaaguccccaggcuccccagcaggcagaagua
ugcaaagcaugcaucucaauuagucagcaaccagguguggaaagucccca
ggcuccccagcaggcagaaguaugcaaagcaugcaucucaauuagucagc
aaccauagucccgccccuaacuccgcccaucccgccccuaacuccgccca
guuacggggucauuaguucauagcccauauauggaguuccgcguuacaua
acuuacgguaaauggcccgccuggcugaccgcccaacgacccccgcccau
ugacgucaauaaugacguauguucccauaguaacgccaauagggacuuuc
cauugacgucaauggguggaguauuuacgguaaacugcccacuuggcagu
acaucaaguguaucauaugccaaguacgcccccuauugacgucaaugacg
guaaauggcccgccuggcauuaugcccaguacaugaccuuaugggacuuu
ccuacuuggcaguacaucuacguauuagucaucgcuauuaccauggugau
gcgguuuuggcaguacaucaaugggcguggauagcgguuugacucacggg
gauuuccaagucuccaccccauugacgucaaugggaguuuguuuuggcac
caaaaucaacgggacuuuccaaaaugucguaacaacuccgccccauugac
gcaaaugggcgguaggcguguacggugggaggucuauauaagcagagcuc
guuuagugaaccgucagaucgccuggagacgccauccacgcuguuuugac
cuccauagaagacaccgggaccgauccagccuccgcggcucgcaucucuc
cuucacgcgcccgccgcccuaccugaggccgccauccacgccgguugagu
cgcguucugccgccucccgccuguggugccuccugaacugcguccgccgu
cuagguaaguuuaaagcucaggucgagaccgggccuuuguccggcgcucc
cuuggagccuaccuagacucagccggcucuccacgcuuugccugacccug
cuugcucaacucuaguucucucguuaacuuaaugagacagauagaaacug
gucuuguagaaacagaguagucgccugcuuuucugccaggugcugacuuc
ucuccccugggcuuuuuucuuuuucucagguugaaaagaagaagacgaag
aagacgaagaagacaaagccgccaccauggaugcaaugaagagagggcuc
ugcugugugcugcugcuguguggagcagucuucguuucgcccagcgguac
cggauccgucgaccaaggcaagcuugugcggcuggacaacaucuacgcca
ucaagaccaacggcaccaacaucuucggcgccauggccaucgacuucuac
gcuaucgccagaaaucugcggagcaugcugucucuggacuaucugcacac
cagcgaagugaagcggaacuuucugcugaucgaguucuucgacgugcugu
acggacugcagagcaacagcacccggaagcacaucgagaacaagaacgcc
uucguguccaucuucaccaacgccagcaucugcaugagcaacuucagcgc
cgaucuguacaaagaguucuucaacaacaccaccaauccgaucgagagcu
uccucgacaucuucaugaugcugaccagccggcggagccucgugaacccu
uggaaaaccggaaucuacagagcccagaccgcucugaucagcuucaucaa
gcaaggcugcuacacuuacauccucagccuccaagccagagagcugcuga
uugccgccuacuacuacuauguggcccaaaucuacagcaaucugacccac
aacaagcaagaguuccaagaggaaaacuucaucaccaaucugagcaacca
gaucacccagaaucugaaggaccagccucugaucuucaagaaucuguuca
ucuacuuuaagaaucucaagagcaagaacauccucgugcggcaccacgcc
uucagcuaucugaccaagaauccucugacacugaacaacagcuacagccc
uccuaagccucugccuagcaucccucugcugccaaacaucccuccacuga
gcacccagaacaucagucugaucgaagugggcuaccccaucagcuucgcc
cggacacugcaaguggccgagacagagaaagaguucaucacgcugcugaa
ucaagcucuggccagcacacagcuguaccgggugcacguguuccggauuc
ugcggaacgagaacacccacagcuuccagaaguaccggcagcacaucuuc
uucuuccaaaacaacgcccuccaagugacauaccggaagcugcgguucug
guucaacgagaaugugaaucuggcuauccccuccgugucuauccccuuuc
ggauccccuucgugauucugacaagcgccaccaucgacacccacaaguac
aacgugaagaucgcccacaucaagagucuggccuccgccauggucaccga
caaagaggccgauaugcugagcuucaucucugccgccgugaacagcaccg
gacugauuggacauguggccuacgccuccaucaucaccgccaugacacau
cugaugguccgagaucaucugaagcacuacacacugaaccaugccuucac
acugucucugaucugcgugaagcaagaauaucugugccuccucaucaacc
ccaagcuggucaccaaguuucugcagcggaacaucaucgaggccuucucc
gccgcucuguccaagaauacugccagcgaucugaaccgguccaacaccga
cgugguguaucugauccccucucugacucugcacacacccauguucuaaa
gaucuuuuucccucugccaaaaauuauggggacaucaugaagccccuuga
gcaucugacuucuggcuaauaaaggaaauuuauuuucauugcaauagugu
guuggaauuuuuugugucucucacucggaaggacauaagggcggcc

1-58. (canceled)

59. A polypeptide comprising: one or more amino acid sequences selected from the group consisting of SEQ ID NOS: 1-67, 70-71, and/or fragments and variants thereof having at least 80% homology to any one of SEQ ID NOS: 1-67, 70-71, wherein said fragments or variants retain MHC binding propensity and the same TCR specificity, and/or retains ASFV activity; or
a nucleic acid encoding said polypeptide.

60. The polypeptide of claim 59, wherein said polypeptide comprises any one of SEQ ID NOS: 1-65 and/or fragments and variants thereof.

61. The polypeptide of claim 59, wherein said polypeptide comprises any one of SEQ ID NOS: 66-67 or 70-71, and/or fragments and variants thereof.

62. The polypeptide or claim 59, wherein said polypeptide has an amino acid sequence at least 80% identical to SEQ ID No: 66.

63. The polypeptide or claim 59, wherein said polypeptide has an amino acid sequence at least 80% identical to SEQ ID No: 67.

64. The polypeptide or claim 59, wherein said polypeptide has an amino acid sequence at least 80% identical to SEQ ID No: 70.

65. The polypeptide or claim 59, wherein said polypeptide has an amino acid sequence at least 80% identical to SEQ ID No: 71.

66. The polypeptide of claim 59, wherein said nucleotide comprises a sequence selected from the group consisting of SEQ ID NOS: 68-69, 72-77, and fragments or variants thereof.

67. The polypeptide of claim 66, wherein said polypeptide retains ASFV activity.

68. The polypeptide or claim 59, wherein the nucleotide has a sequence at least 80% identical to SEQ ID No: 68 provided said polypeptide encoded by said nucleic retains anti-ASFV activity.

69. The polypeptide or claim 59, wherein the nucleotide has a sequence at least 80% identical to SEQ ID No: 69 provided said polypeptide encoded by said nucleic retains anti-ASFV activity.

70. The polypeptide or claim 59, wherein the nucleotide has a sequence at least 80% identical to SEQ ID No: 72 provided said polypeptide encoded by said nucleic retains anti-ASFV activity.

71. The polypeptide or claim 59, wherein the nucleotide has a sequence at least 80% identical to SEQ ID No: 73 provided said polypeptide encoded by said nucleic retains anti-ASFV activity.

72. The polypeptide or claim 59, wherein the nucleotide has a sequence at least 80% identical to SEQ ID No: 74, 75, 76, or 77 provided said polypeptide encoded by said nucleic retains anti-ASFV activity.

73. The polypeptide of claim 59, further comprising a pharmaceutically-acceptable excipient, carrier, or adjuvant.

74. A method for inducing an immune response against ASFV and/or a related disease caused by ASFV infection in a subject in need thereof, the method comprising administering to the subject a therapeutically effective amount of a polypeptide or nucleotide according to claim 59.

75. The method of claim 74, wherein said polypeptide or nucleotide further comprises a pharmaceutically-acceptable excipient, carrier, or adjuvant.

76. The method of claim 74, further comprising administering to the subject an African Swine Fever Virus, wherein the virus is a live attenuated virus or inactivated virus.

77. A chimeric or fusion polypeptide comprising one or more amino acid sequences selected from the group consisting of SEQ ID NOS: 1-67, 70-71, and/or fragments and variants thereof having at least 80% homology to any one of SEQ ID NOS: 1-67, 70-71, wherein said fragments or variants retain MHC binding propensity and the same TCR specificity, and/or retains ASFV activity,
wherein said polypeptide is joined, linked, or inserted into a heterologous polypeptide.

78. The polypeptide of claim 77, wherein said polypeptide comprises any one of SEQ ID NOS: 66-67 or 70-71, and/or fragments and variants thereof.
